(19) United States
(12) Patent Application Publication
(10) Pub. No.: US 2019/0119403 A1
(43) Pub. Date: Apr. 25, 2019

(54) METHODS OF CONSTRUCTING IMMUNOGLOBULIN FUSION PROTEINS INHIBITING CATHEPSIN B AND COMPOSITIONS THEREOF

(71) Applicants: Yong Zhang, Los Angeles, CA (US); Zhefu Dai, Los Angeles, CA (US); University of Southern California, Los Angeles, CA (US)

(72) Inventors: Yong Zhang, Los Angeles, CA (US); Zhefu Dai, Los Angeles, CA (US)

(21) Appl. No.: 16/090,148

(22) PCT Filed: Mar. 31, 2017
(86) PCT No.: PCT/US17/25536

§ 371 Date: Sep. 28, 2018

Related U.S. Application Data
(60) Provisional application No. 62/316,458, filed on Mar. 31, 2016.

(51) Int. Cl. C07K 16/40; C07K 16/32; C12N 9/50

(52) U.S. Cl. CPC C07K 16/40; C07K 16/32; C07K 2317/24; C07K 2319/30; C07K 2319/50; C12N 9/50

(57) ABSTRACT

Cathepsin B plays a crucial role in promoting cancer cells invasion and metastasis. Through structure-guided rational design, Applicants have generated monoclonal antibodies specifically inhibiting human cathepsin B proteolytic activity. These novel antibody inhibitors provide potential potent anti-metastasis therapeutics with excellent safety profiles.

### CROSS REFERENCE TO RELATED APPLICATIONS

This application claims priority under 35 U.S.C. § 119(e) to U.S. Provisional Application No. 62/316,458, filed Mar. 31, 2016, the content of which is hereby incorporated by reference in its entirety.

### BACKGROUND

Cathepsin B, a cysteine protease, plays a crucial role in promoting cancer development and metastasis. It is overexpressed by malignant cancer cells and secreted into extracellular region. By activating the plasminogen activator and the subsequent proteolytic cascade, cathepsin B secretion incites degradation and destruction of the extracellular matrices surrounding cancer cells, enabling invasion of the basement membrane and spread of cancer to distant organs and tissues. Up-regulated cathepsin B also leads to increased expression of vascular endothelial growth factor (VEGF), promoting tumor angiogenesis. Previous studies revealed that suppression of cathepsin B in cancer cells led to inhibition of tumorigenicity and metastasis. Serum levels of cathepsin B in cancer patients significantly correlate with survival rates. Thus, inhibitors against cathepsin B protease activity can potentially restrict and/or arrest cancer metastasis and have been long considered to be promising drug candidates. However, recently developed small-molecule protease inhibitors often cause severe side effects due to their significant off-target effects, which is restricting clinical use. In comparison, monoclonal antibodies are characterized by exquisite specificity and tight-binding affinity to their cognate antigens, making antibody-based protease inhibitors ideal drug candidates. Monoclonal antibodies specifically inhibiting cathepsin B can become potent anti-metastasis therapeutics with excellent safety profiles.

### SUMMARY OF THE DISCLOSURE

In order to address the needs in the art, the applicants provide a recombinant cathepsin B antibody that inhibits proteolytic activity of human cathepsin B. In a further aspect, the antibody inhibits the proteolytic activity of human cathepsin B in a dose-dependent fashion. The antibody comprises, or alternatively consists essentially of, or yet further comprises the recombinant light and heavy chains as described herein. Non-limiting examples of the polypeptide and polynucleotide sequences of the antibody heavy and light chains also are provided herein.

Further provided are compositions comprising, or alternatively consisting essentially or, or yet further consisting of, the recombinant polynucleotide, antibody, antibody fragment, vector or host cell or precursor for use in making the antibody, and a detectable label and/or a carrier, e.g. a solid or liquid carrier. In one aspect, the carrier is a pharmaceutically acceptable carrier.

The compositions can be used for screening and purification of naturally occurring products by reliance on their inherent properties to recognize and bind binding partners. They also can be used in vitro and in vivo to inhibit human cathepsin B activity or treating a condition or disease linked to cathepsin B expression in a subject, by contacting the cathepsin B or a sample suspected of containing the cathepsin B with an effective amount of the recombinant antibody. A non-limiting example of such a condition is metastatic cancer.

In one particular aspect and non-limiting example of this invention, described herein is a propeptide genetically fused to a recombinant HER-2 antibody having stefin A sequences. In one aspect the disclosure, provide herein is an N-terminus of the light chain of a humanized anti-HER2 receptor monoclonal antibody trastuzumab, (referred to hereafter by its traded name Herceptin). The full-length Herceptin-propeptide IgG fusion was expressed in freestyle HEK 293 cells and characterized by SDS-PAGE gel stained with coomassie blue. The antibody fusion showed a molecular weight matching to the calculated one.

Using recombinant human cathepsin B and a fluorogenic peptide substrate, inhibition activity of the generated antibody was characterized. The generated antibody-propeptide IgG fusion potently inhibits the proteolytic activity of human cathepsin B in a dose-dependent manner. The structures of propeptide and Herceptin are available and attached. The structural model and sequences (DNA and amino acids) of the invented propeptide-antibody fusion are also attached.

### BRIEF DESCRIPTION OF THE DRAWINGS

The drawings as presented herein set forth various aspects of the invention as further described herein.

FIG. 1 depicts cathepsin B in zymogen form, where the highlighted (dark gray) and isolated structure is the propeptide. The propeptide in cathepsin zymogen is a potent inhibitor for active cathepsin B.

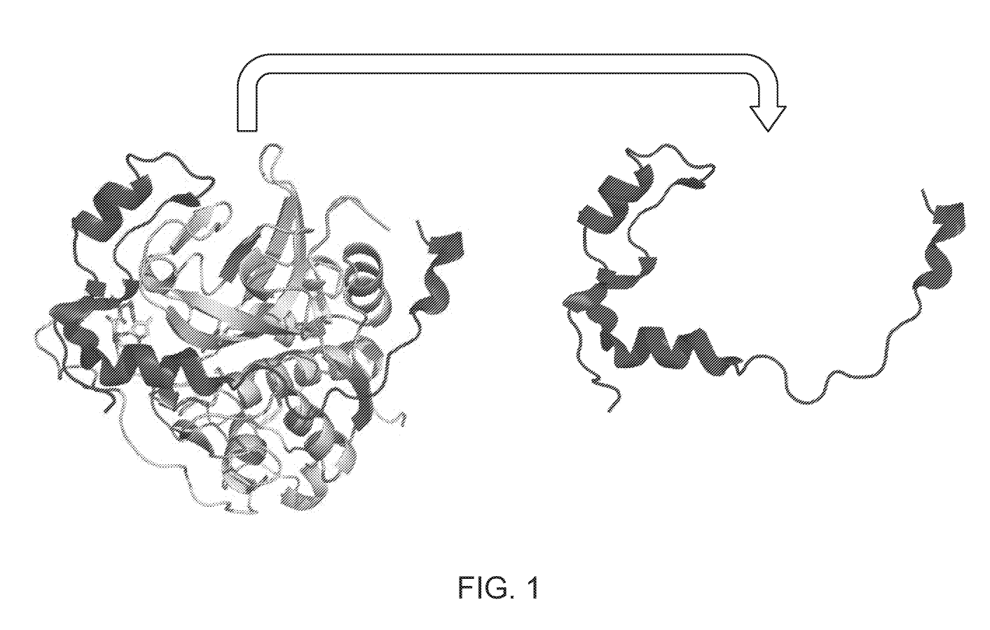

FIG. 2 depicts the secondary and tertiary structure of an exemplary N-fusion antibody based inhibitor for cathepsin B, wherein the circled and zoomed in structure comprises the propeptide.

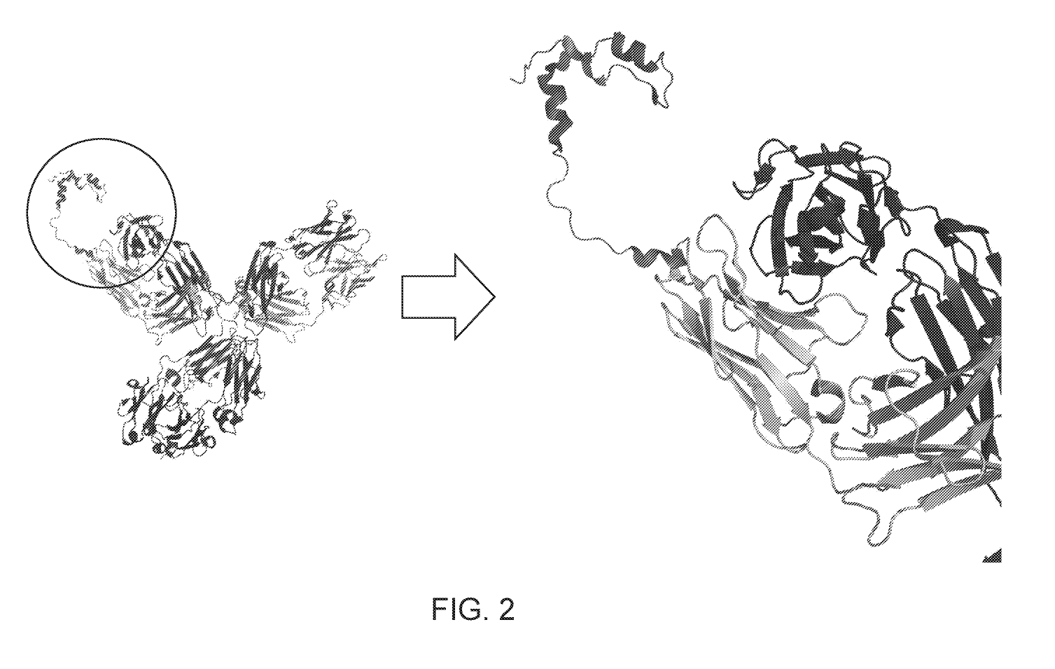

FIG. 3 shows the secondary and tertiary structure of propeptide and the potential Factor Xa cleavage site for propeptide fused to the CDR loop of an exemplary antibody.

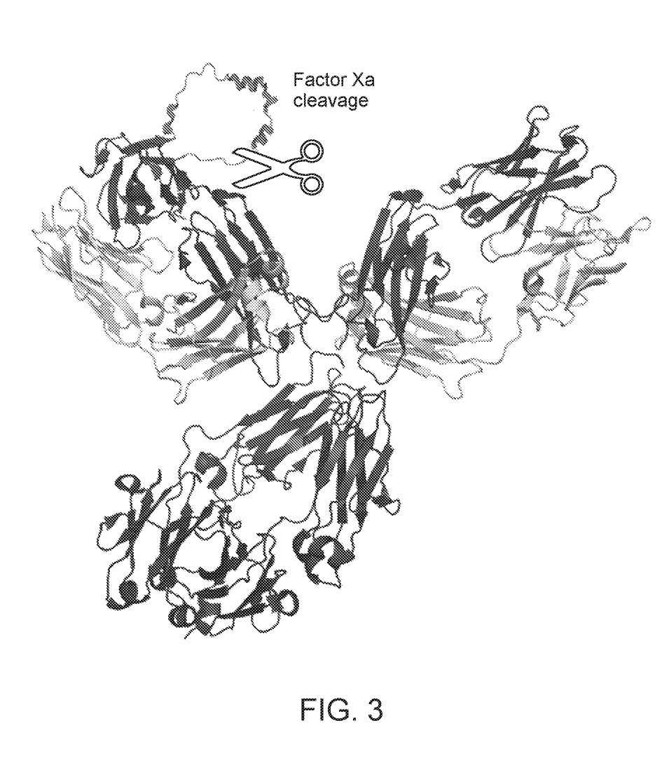

FIG. 4 shows the results of Factor Xa cleavage of propeptide—release of the propeptide N-terminus.

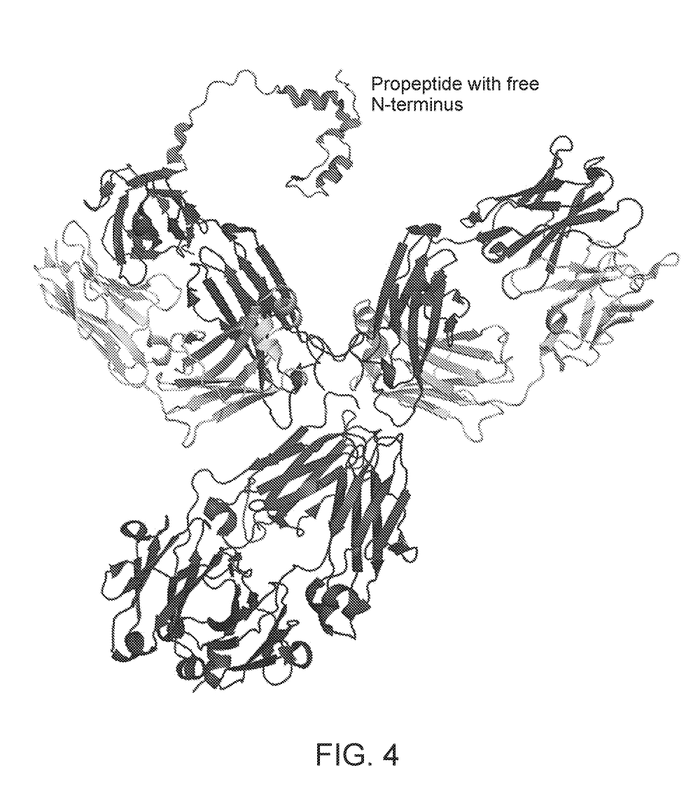

FIG. 5 is an SDS page gel showing purification of antibody with propeptide fused to the light chain N-terminus of an exemplary antibody.

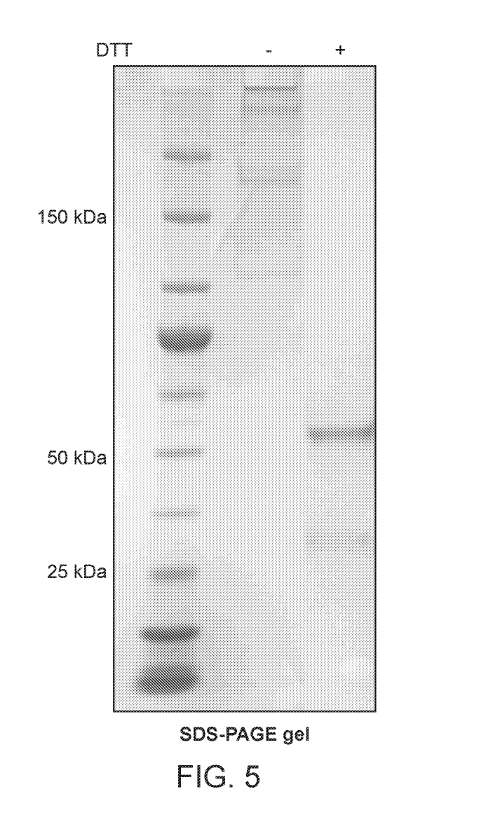

FIG. 6 depicts the calculation of the inhibition constant proteolytic activity at various concentrations of the N-fusion antibody exemplified in the figures above antibody (where there was no preincubation).

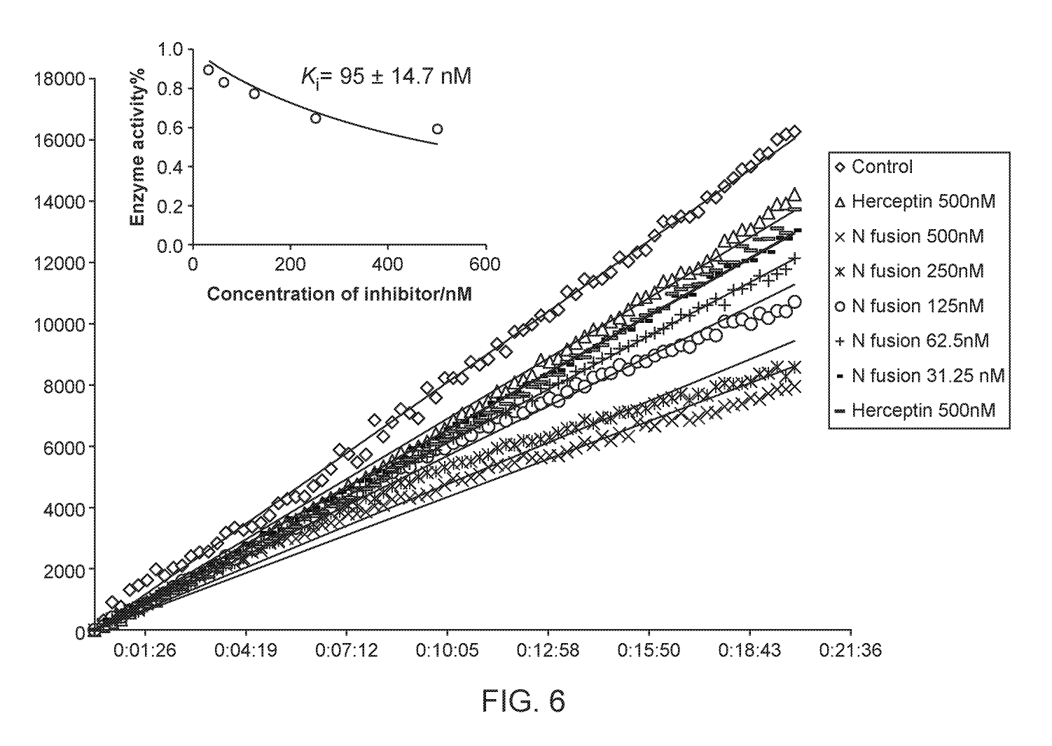

FIG. 7 depicts the calculation of the inhibition constant proteolytic activity at various concentrations of the N-fusion antibody exemplified in the figures above antibody (where there was 10 minutes of preincubation).

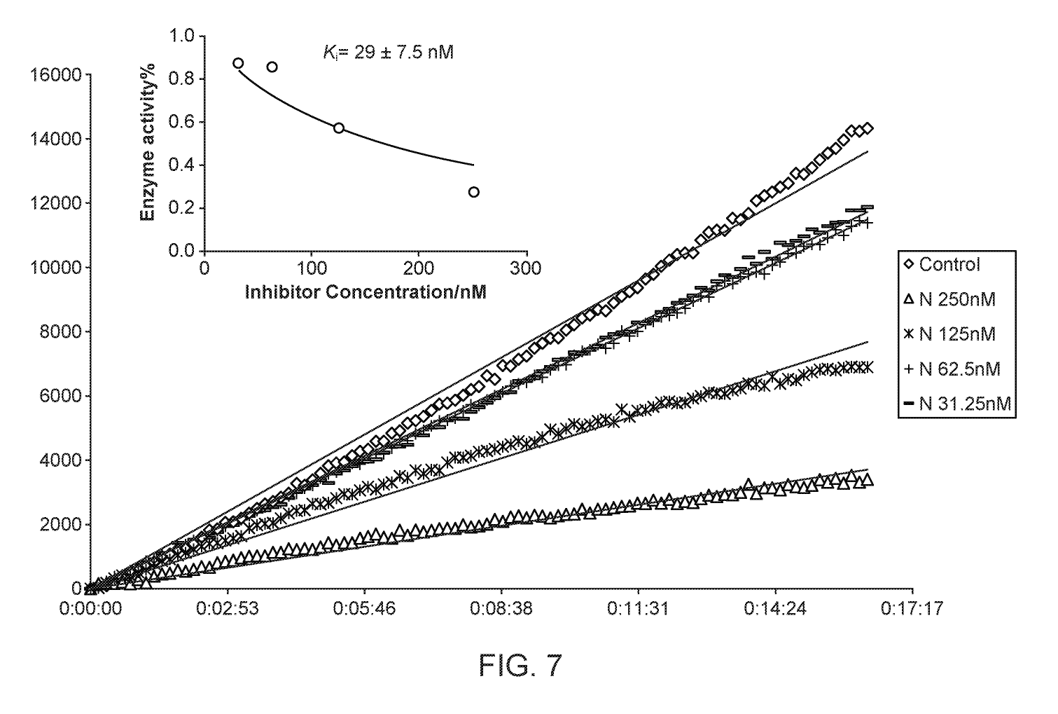

FIG. 8 depicts the secondary and tertiary structure of stefin A (left) and its loop structures interacting with cathepsin B (right); stefin A is a natural inhibitor to cathepsins. This interaction serves as the basis for the stefin A based antibody design disclosed herein.

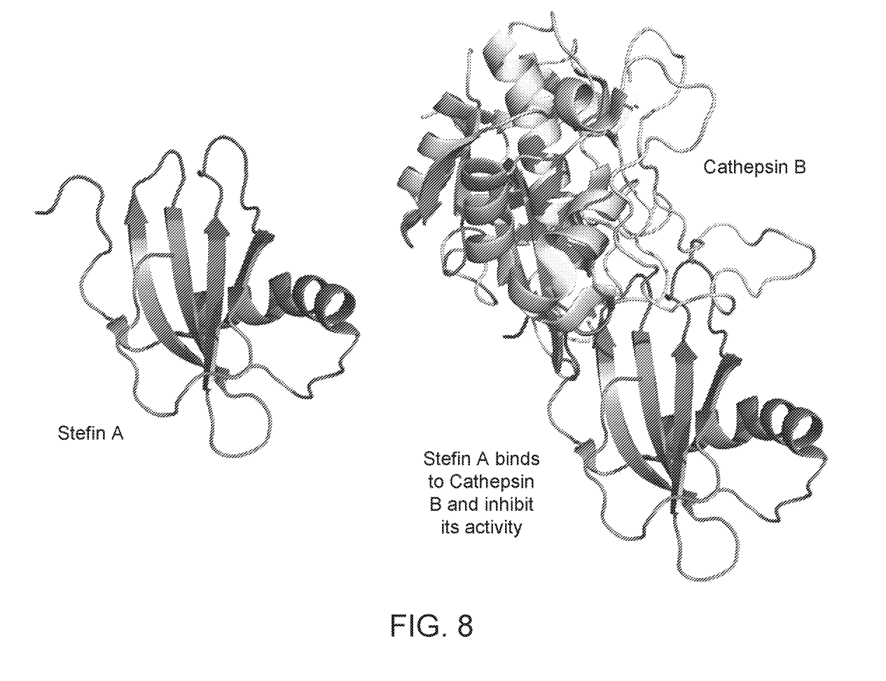

FIG. 9 shows the generation 1 antibody design in which the three loops of the Herceptin antibody (right) may be arranged in a similar manner to the three loops on Stefin A (left).

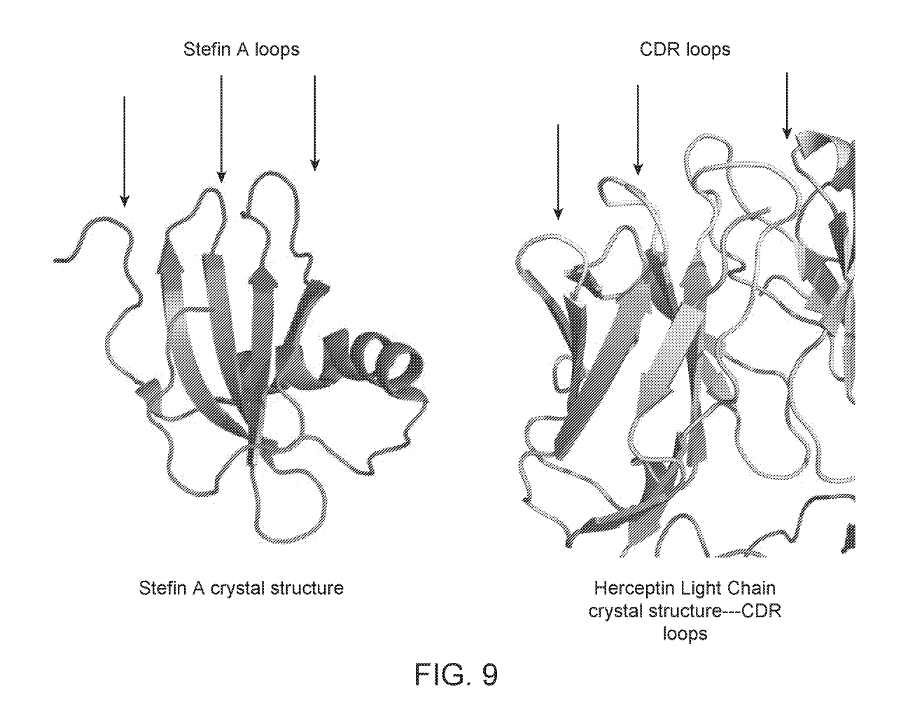

FIG. 10 shows a schematic of the generation 1 Fab design, wherein the VL CDR loops of Herceptin are modified based on the Stefin A structure.

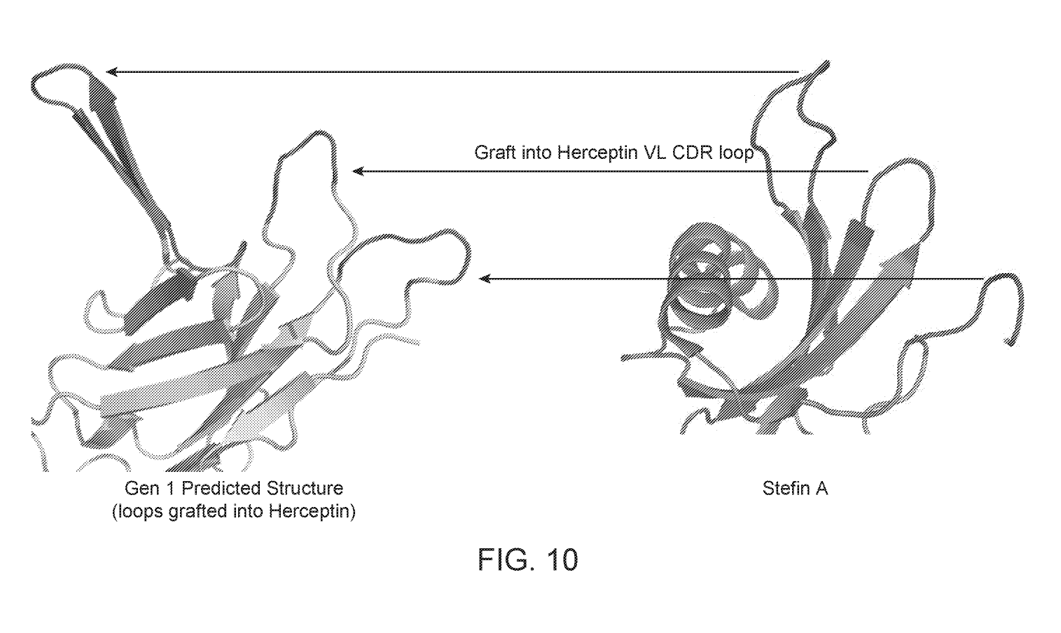

FIG. 11 shows an SDS page gel showing purification of stefin A-derived antibody Fab.

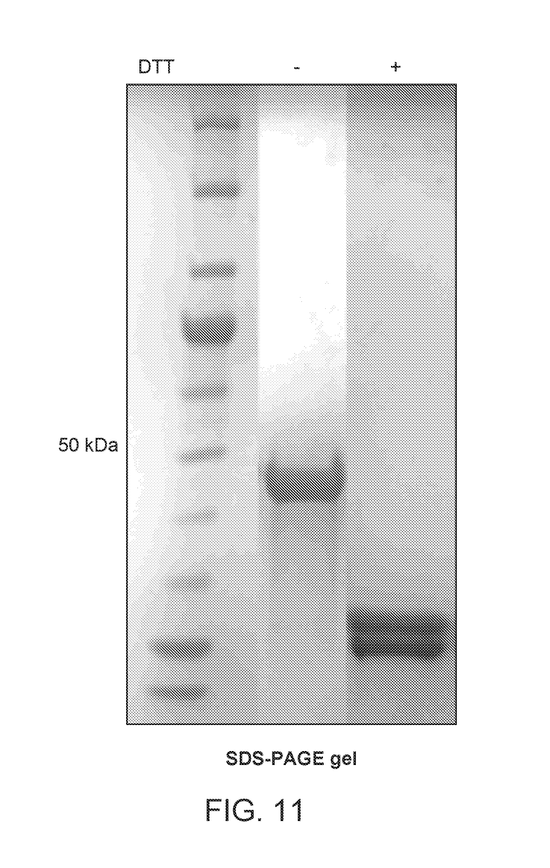

FIG. 12 depicts the calculation of the inhibition constant proteolytic activity at various concentrations of the stefin A-derived antibody Fab exemplified in the figures above antibody.

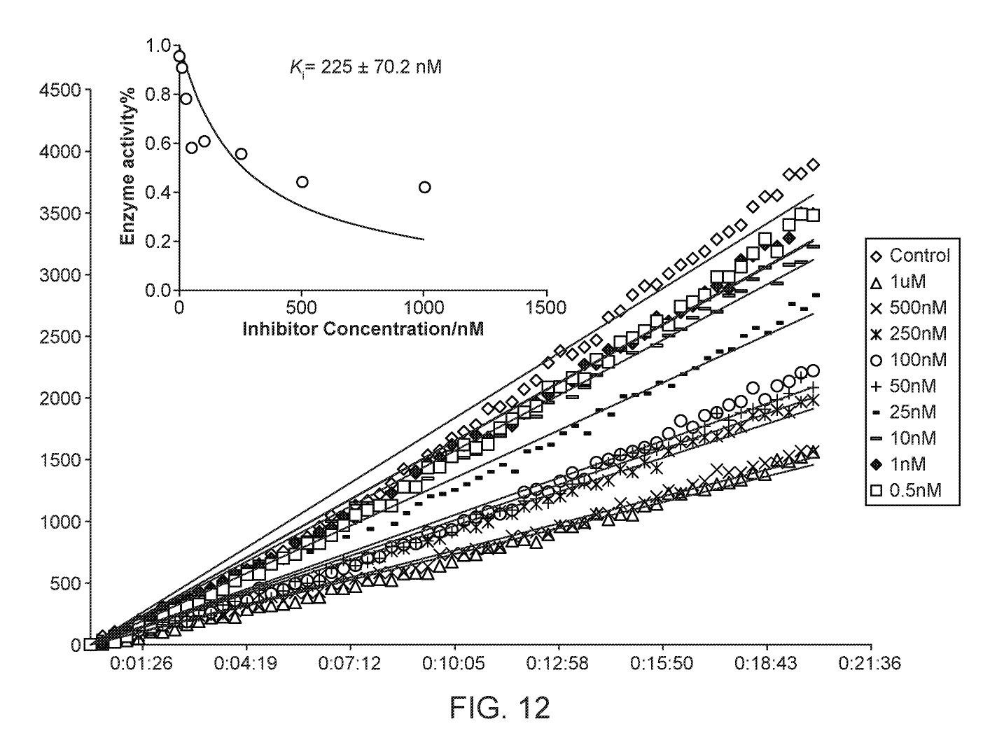

FIG. 13 depicts the structure of propeptide (right) bound to cathepsin B (right), wherein the propeptide is dark gray.

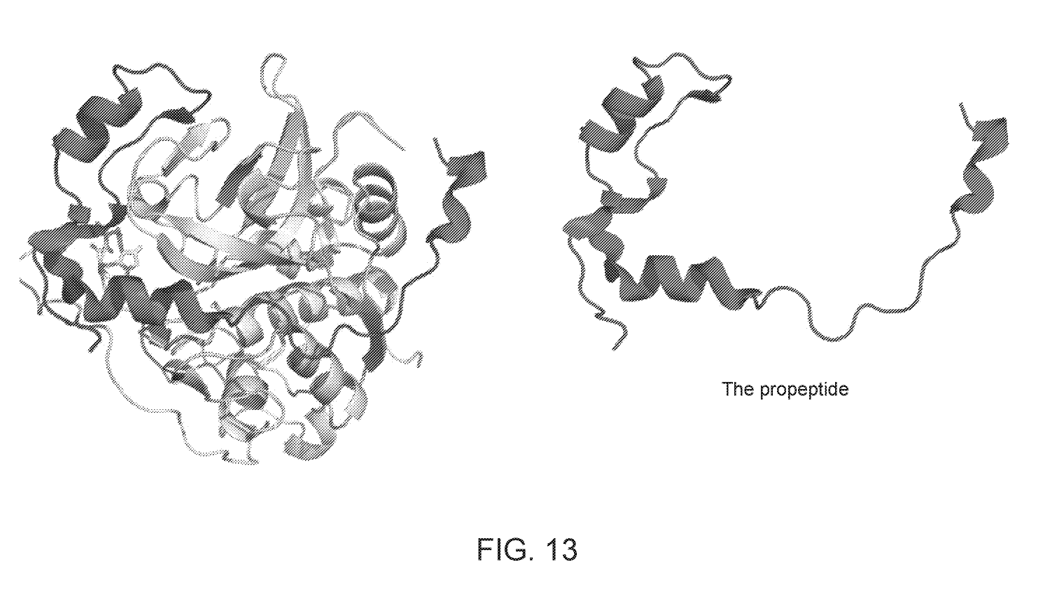

FIG. 14 depicts the secondary and tertiary structure of an exemplary Herceptin antibody.

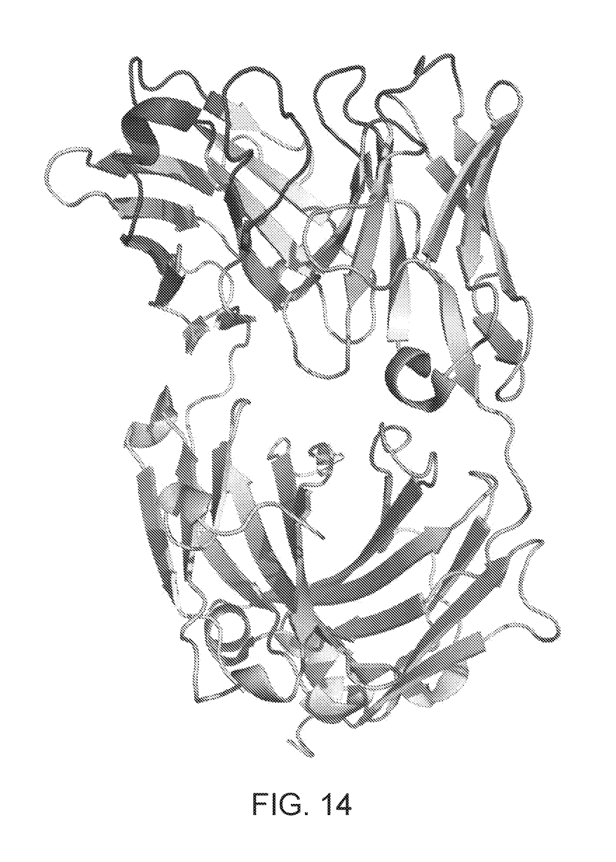

FIG. 15 depicts the secondary and tertiary structure of a propeptide antibody fusion protein.

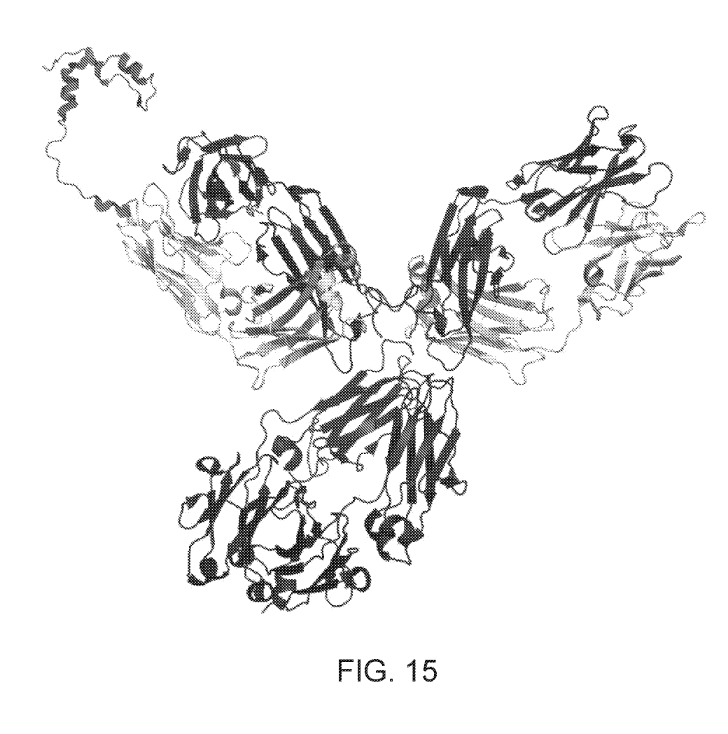

FIG. 16 depicts the secondary and tertiary structure of stefin A.

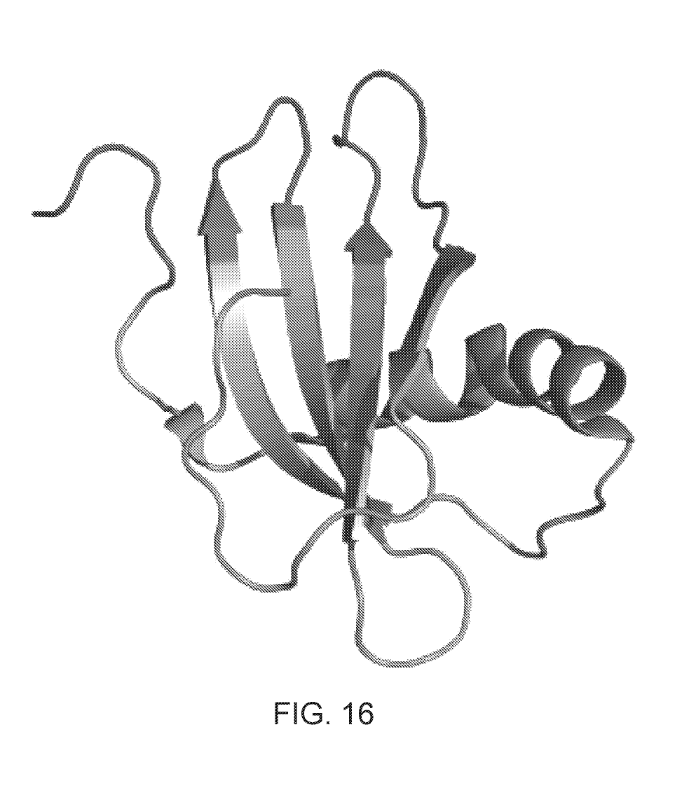

FIG. 17 depicts the secondary and tertiary structure of an exemplary Herceptin antibody.

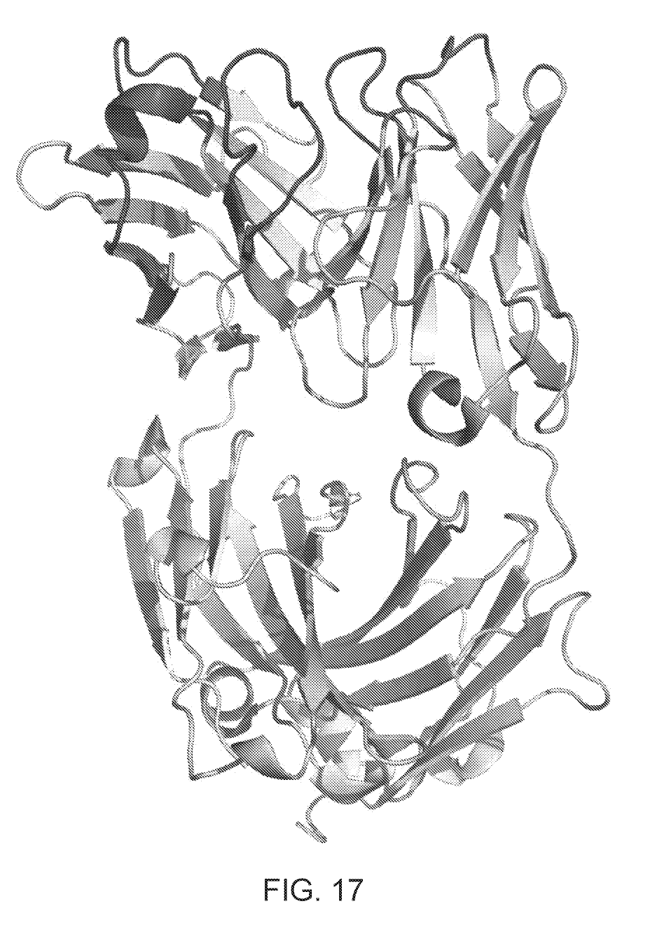

FIG. 18 provides the secondary and tertiary structure of a portion of an exemplary stefin A-derived antibody, wherein the dark gray represents the portions of the antibody derived from stefin A.

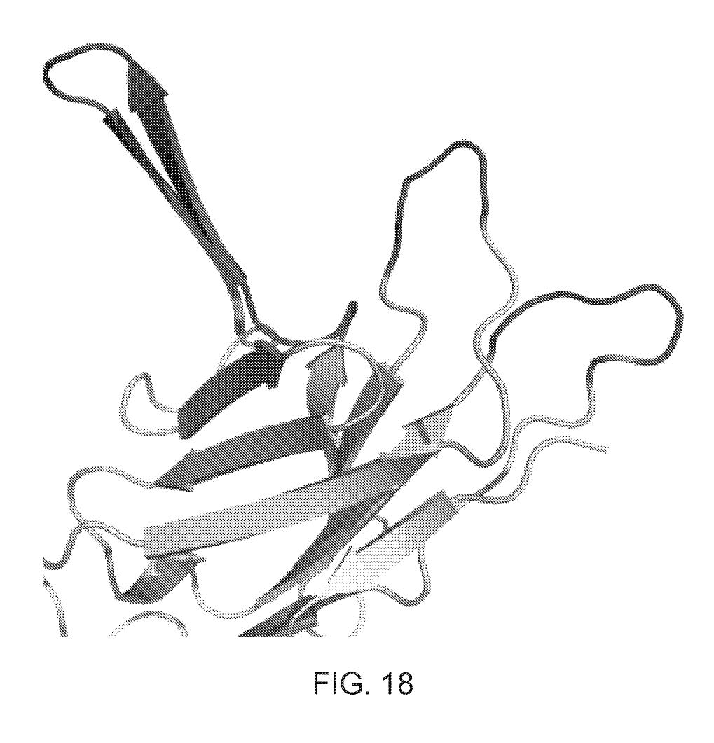

FIG. 19 provides the secondary and tertiary structure of a portion of an exemplary stefin A-derived antibody with the inhibition loop of stefin A (dark gray) fused to the loop x (pale gray) of Herceptin.

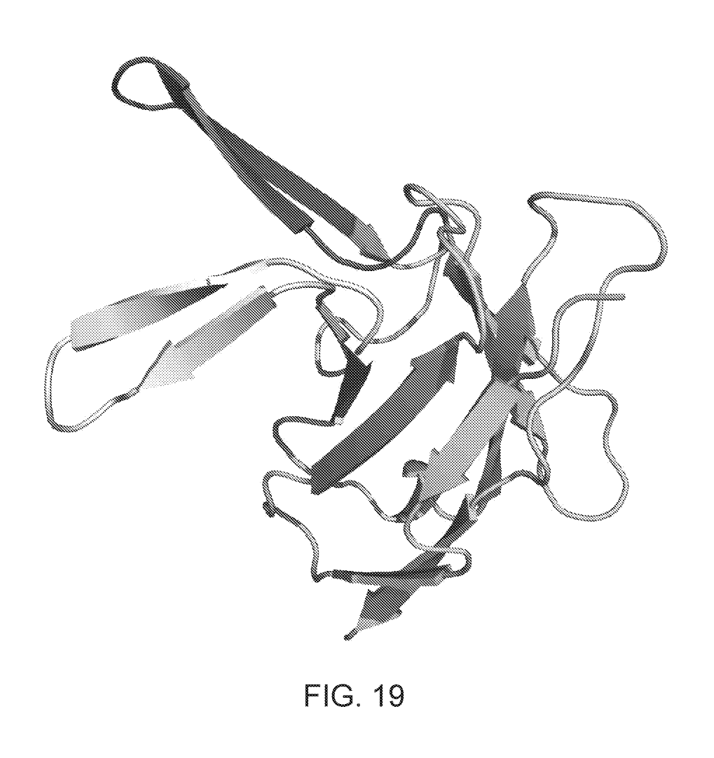

FIG. 20 depicts the secondary and tertiary structure of a propeptide antibody fusion protein, where the propeptide is fused to the heavy chain N-terminus.

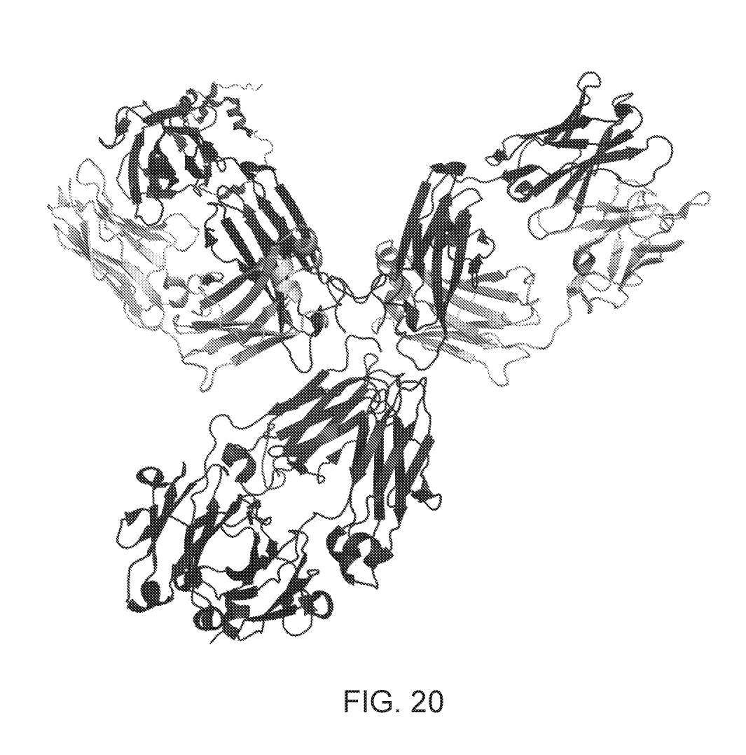

FIG. 21 depicts the secondary and tertiary structure of a propeptide antibody fusion protein, where the propeptide is fused to the heavy chain C-terminus.

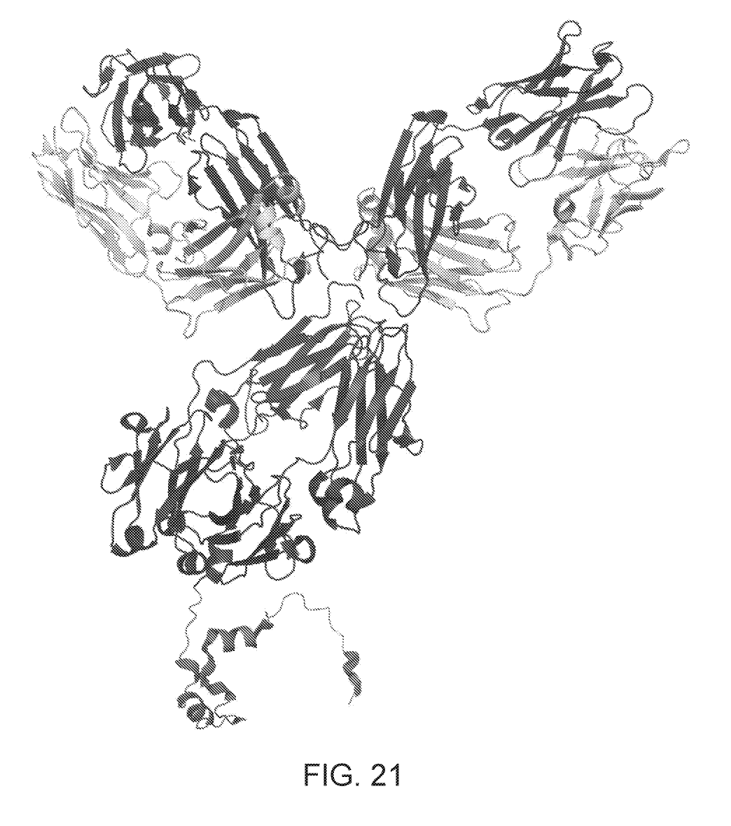

### DETAILED DESCRIPTION

Unless defined otherwise, all technical and scientific terms used herein have the same meanings as commonly understood by one of ordinary skill in the art to which this disclosure belongs. All nucleotide sequences provided herein are presented in the 5′ to 3′ direction. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3^{rd} edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

A “subject” of diagnosis or treatment is a cell or an animal such as a mammal, or a human. Non-human animals subject to diagnosis or treatment and are those subject to infections or animal models, for example, simians, murines, such as, rats, mice, chinchilla, canine, such as dogs, leporids, such as rabbits, livestock, sport animals, and pets.

Cathepsin B, as used herein, refers to a protein referred to by that name and/or equivalents thereof. Cathepsin B is lysosomal cysteine proteases known to play a role in intracellular proteolysis. Cathepsin B exists in both an active and zymogen form—featuring a propeptide disclosed herein. Upregulation of cathepsin B is associated with premalignant lesions, as well as a variety of pathological conditions and cancers. A non-limiting exemplary sequence of cathepsin B can be found under UNIPROT Ref. No. Q6LAF9 (uniprot.org/uniprot/Q6LAF9.html, last accessed Mar. 27, 2017); the human sequence of which is reproduced herein below:

CGSCWAFGAV EAISDRICIH TNVSVEVSAE DLLTCCGSMC
GDGCNGGYPA EAWNFWTRKG LVSGGLYESH VGCRPYSIPP
CEHHVNGSRP PCTGEGDTPK CSKICEPGYS PTYKQDKHYG
YDSYSVSNSE KDIMAEIYKN GPVEGAFSVY SDFLLYKSGV
YQHVTGEMMG GHAIRILGWG VENGTPYWLV ANSWN

“Cathepsin B activity” is intended to include clinical and sub-clinical effects of the protein. As reported by Gondi and Rao (2013) “Cathepsin B as a Cancer Target” Expert. Opin. Ther. Targets March 17(3):281-291, cathepsin B is known to be of significant importance to cancer therapy and it is involved in various pathologies and oncogenic processes in humans. Overexpression of cathepsin B is correlated with invasive and metastatic phenotypes in cancers. Abnormal regulation of cathepsin B causes cells to acquire an oncogenic character. The proteolytic nature of cathepsin B has been attributed to the infiltrative nature of tumor cells, and it has been shown that cathepsin B is secreted into the extracellular matrix (ECM), thereby facilitating its destruction. Suppression of the proteolytic activity of cathepsin B retards the infiltrative behavior of tumor cells and tumor growth.

“A condition mediated by cathepsin B activity” intends abnormal conditions related to the expression of the protein, non-limiting examples include infection, inflammation, cancer, metastates, metastatic potential of cancer cell, melanoma, breast cancer, oral cancer, ovarian cancer, pancreatic cancer, lung cancer, liver cancer, rheumatoid arthritis, and osteoarthrisis.

HER2 or human epidermal growth factor receptor 2, is a gene that is known to play a role in the development of breast cancer. The HER2 gene makes HER2 proteins. HER2 proteins are receptors on breast cells. The protein coding sequence of the gene is known in the art, e.g., see genecards.org/cgi-bin/carddisp.pl?gene=ERBB2, last accessed on Mar. 28, 2016.

Stefin A is also known as cystatin A (CSTA). As reported by the Atlas of Genetics and Cytogenitcs in Oncology and Haematology (see atlasgeneticsoncology.org/Genes/GC_CSTA.htmlatla). The protein belongs to the cystatin superfamily of cysteine protease inhibitors. The lack of a signal sequence and disulfide bonds makes stefins distinct from other members of the cystatin superfamily. Human stefin A is a single chain protein consisting of 98 amino acid residues, with a molecular mass of 11 kDa. Stefin A is an acidic protein with pI values between 4.5-5.0. Like other members of the cystatin superfamily, stefin A is reversible and competitive inhibitor of cysteine proteases, particularly cathepsin L and cathepsin S with Ki values in the picomolar range whereas cathpsin B inhibition is weaker (Ki 10⁻⁸M). The gene for human stefin A is located on chromosome 3q21 and it comprises three exons of 11 base pairs, 111 bp, 102 bp and 226 bp in length, while the lengths of the 1st and 2nd intron are approximately 14 Kbp and 4 Kbp, respectively. The conserved sequence of QVVAG is encoded in the 2nd exon and is not inserted by any introns. The transcript length of stefin A mRNA is 294 bps. Binding sites for AP-2 (Activating Protein 2) and Sp1 (Selective Promoter Factor 1) regulatory elements are present in the promoter region and an AP-1 (Activating Protein 1) binding site in the 1st intron. Human stefin A is reported to exhibit a high degree of homology to other cysteine protease inhibitors of the cystatin superfamily which includes human stefin B and the homologues in other species such as cystatins alpha and beta in rat, bovine thymus stefin C, porcine thymus stefins D1 and D2, mouse stefins A(1-4) and others. A cDNA coding for stefin A is found under GenBank Accession No. AK291308 (copied below):

| | |
|---|---|
| 1 | actttggttc cagcatcctg tccagcaaag aagcaatcag |
| | ccaaaatgat acctggaggc |
| 61 | ttatctgagg ccaaacccgc cactccagaa atccaggaga |
| | ttgttgataa ggttaaacca |
| 121 | cagcttgaag aaaaaacaaa tgagacttac ggaaaattgg |
| | aagctgtgca gtataaaact |
| 181 | caagttgttg ctggaacaaa ttactacatt aaggtacgag |
| | caggtgataa taaatatatg |
| 241 | cacttgaaag tattcaaaag tcttcccgga caaaatgagg |
| | acttggtact tactggatac |
| 301 | caggttgaca aaaacaagga tgacgagctg acgggctttt |
| | agcagcatgt acccaaagtg |
| 361 | ttctgattcc ttcaactggc tactgagtca tgatccttgc |
| | tgataaatat aaccatcaat |
| 421 | aaagaagcat tcttttccaa aaaaaaaaaa aaaagaaaaa |
| | aaaaaaaaaa aaaaagtggc |
| 481 | gctgggcagc gcgggtccca accagaaacc cgcacaggcg |
| | ac |

wherein the Stefin A sequence can be found from nucleic acids 46-342 and translated as:

MIPGGLSEAKPATPEIQEIVDKVKPQLEEKTNETYGKLEAVQYKTQVVAG
TNYYIKVRAGDNKYMHLKVFKSLPGQNEDLVLTGYQVDKNKDDELTGF

The term “protein”, “peptide” and “polypeptide” are used interchangeably and in their broadest sense to refer to a compound of two or more subunit amino acids, amino acid analogs or peptidomimetics. The subunits may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “isolated” or “recombinant” as used herein with respect to nucleic acids, such as DNA or RNA, refers to molecules separated from other DNAs or RNAs, respectively that are present in the natural source of the macromolecule as well as polypeptides. The term “isolated or recombinant nucleic acid” is meant to include nucleic acid fragments which are not naturally occurring as fragments and would not be found in the natural state. The term “isolated” is also used herein to refer to polynucleotides, polypeptides and proteins that are isolated from other cellular proteins and is meant to encompass both purified and recombinant polypeptides. In other embodiments, the term “isolated or recombinant” means separated from constituents, cellular and otherwise, in which the cell, tissue, polynucleotide, peptide, polypeptide, protein, antibody or fragment(s) thereof, which are normally associated in nature. For example, an isolated cell is a cell that is separated from tissue or cells of dissimilar phenotype or genotype. An isolated polynucleotide is separated from the 3′ and 5′ contiguous nucleotides with which it is normally associated in its native or natural environment, e.g., on the chromosome. As is apparent to those of skill in the art, a non-naturally occurring polynucleotide, peptide, polypeptide, protein, antibody or fragment(s) thereof, does not require “isolation” to distinguish it from its naturally occurring counterpart.

It is to be inferred without explicit recitation and unless otherwise intended, that when the present disclosure relates to a polypeptide, protein, polynucleotide or antibody, an equivalent or a biologically equivalent of such is intended within the scope of this disclosure. As used herein, the term “biological equivalent thereof” is intended to be synonymous with “equivalent thereof” when referring to a reference protein, antibody, fragment, polypeptide or nucleic acid, intends those having minimal homology while still maintaining desired structure or functionality. Unless specifically recited herein, it is contemplated that any polynucleotide, polypeptide or protein mentioned herein also includes equivalents thereof. In one aspect, an equivalent polynucleotide is one that hybridizes under stringent conditions to the polynucleotide or complement of the polynucleotide as described herein for use in the described methods. In another aspect, an equivalent antibody or antigen binding polypeptide intends one that binds with at least 70%, or alternatively at least 75%, or alternatively at least 80%, or alternatively at least 85%, or alternatively at least 90%, or alternatively at least 95% affinity or higher affinity to a reference antibody or antigen binding fragment. In another aspect, the equivalent thereof competes with the binding of the antibody or antigen binding fragment to its antigen in a competitive ELISA assay. In some aspects, an equivalent antibody or antigen binding polypeptide intends one that binds with the same or greater affinity compared to reference antibody or antigen binding fragment. In some aspects, an equivalent antibody or antigen binding polypeptide intends one that have higher affinity for the epitope to which the reference antibody or antigen binding fragment binds than for the whole antigen. In another aspect, an equivalent intends at least about 80% homology or identity and alternatively, at least about 85%, or alternatively at least about 90%, or alternatively at least about 95%, or alternatively 98% percent homology or identity and exhibits substantially equivalent biological activity to the reference protein, polypeptide or nucleic acid. In some aspects, an equivalent intends a protein, polypeptide or nucleic acid that has an identical or substantially equivalent secondary and/or tertiary structure as the reference protein, polypeptide or nucleic acid and exhibits substantially equivalent biological activity to the reference protein, polypeptide or nucleic acid.

A polynucleotide or polynucleotide region (or a polypeptide or polypeptide region) having a certain percentage (for example, 80%, 85%, 90%, or 95%) of “sequence identity” to another sequence means that, when aligned, that percentage of bases (or amino acids) are the same in comparing the two sequences. The alignment and the percent homology or sequence identity can be determined using software programs known in the art, for example those described in Current Protocols in Molecular Biology (Ausubel et al., eds. 1987) Supplement 30, section 7.7.18, Table 7.7.1. In certain embodiments, default parameters are used for alignment. A non-limiting exemplary alignment program is BLAST, using default parameters. In particular, exemplary programs include BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: ncbi.nlm.nih.gov/cgi-bin/BLAST. Sequence identity and percent identity were determined by incorporating them into clustalW (available at the web address:align.genome.jp, last accessed on Mar. 7, 2011.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

“Homology” or “identity” or “similarity” can also refer to two nucleic acid molecules that hybridize under stringent conditions.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogsteen binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Examples of stringent hybridization conditions include: incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include: incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

As used herein, the term “vector” refers to a nucleic acid construct deigned for transfer between different hosts, including but not limited to a plasmid, a virus, a cosmid, a phage, a BAC, a YAC, etc. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector.

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, the term “animal” refers to living multi-cellular vertebrate organisms, a category that includes, for example, mammals and birds. The term “mammal” includes both human and non-human mammals.

As used herein, the term “antibody” collectively refers to immunoglobulins or immunoglobulin-like molecules including by way of example and without limitation, IgA, IgD, IgE, IgG and IgM, combinations thereof, and similar molecules produced during an immune response in any vertebrate, for example, in mammals such as humans, goats, rabbits and mice, as well as non-mammalian species, such as shark immunoglobulins. Unless specifically noted otherwise, the term “antibody” includes intact immunoglobulins and “antibody fragments” or “antigen binding fragments” that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴M⁻¹ greater or at least 10⁵ M⁻¹ greater than a binding constant for other molecules in a biological sample and/or antibodies or antibody fragments having a dissociation or inhibition constant of less than, or at most, 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M). The term “antibody” also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

As used herein, the term “antigen” refers to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens. As used herein, the term “epitope” refers to the part of an antigen molecule to which an antibody or antigen binding fragment binds. The term epitope encompasses both the particular sequence (e.g. of residues, molecules, etc.) and the secondary and/or tertiary structure of this fragment of the antigen. The term “conformational epitope” is used herein when specifying solely the secondary and/or tertiary aspects of the epitope. In some aspects, the conformational epitope intends this structure without regard to the level of sequence homology.

As used herein, the term “antigen binding domain” refers to any protein or polypeptide domain that can specifically bind to an antigen target.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)₂, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues.

The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

As used herein, “monoclonal antibody” refers to an antibody obtained from a substantially homogeneous antibody population. Monoclonal antibodies are highly specific, as each monoclonal antibody is directed against a single determinant on the antigen. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like.

Monoclonal antibodies may be generated using hybridoma techniques or recombinant DNA methods known in the art. A hybridoma is a cell that is produced in the laboratory from the fusion of an antibody-producing lymphocyte and a non-antibody producing cancer cell, usually a myeloma or lymphoma. A hybridoma proliferates and produces a continuous sample of a specific monoclonal antibody. Alternative techniques for generating or selecting antibodies include in vitro exposure of lymphocytes to antigens of interest, and screening of antibody display libraries in cells, phage, or similar systems.

The term “human antibody” as used herein, is intended to include antibodies having variable and constant regions derived from human germline immunoglobulin sequences. The human antibodies disclosed herein may include amino acid residues not encoded by human germline immunoglobulin sequences (e.g., mutations introduced by random or site-specific mutagenesis in vitro or by somatic mutation in vivo). However, the term “human antibody” as used herein, is not intended to include antibodies in which CDR sequences derived from the germline of another mammalian species, such as a mouse, have been grafted onto human framework sequences. Thus, as used herein, the term “human antibody” refers to an antibody in which substantially every part of the protein (e.g., CDR, framework, C_{L}, C_{H} domains (e.g., C_{H1}, C_{H2}, C_{H3}), hinge, (VL, VH)) is substantially non-immunogenic in humans, with only minor sequence changes or variations. Similarly, antibodies designated primate (monkey, baboon, chimpanzee, etc.), rodent (mouse, rat, rabbit, guinea pig, hamster, and the like) and other mammals designate such species, sub-genus, genus, sub-family, family specific antibodies. Further, chimeric antibodies include any combination of the above. Such changes or variations optionally retain or reduce the immunogenicity in humans or other species relative to non-modified antibodies. Thus, a human antibody is distinct from a chimeric or humanized antibody. It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

As used herein, a human antibody is “derived from” a particular germline sequence if the antibody is obtained from a system using human immunoglobulin sequences, e.g., by immunizing a transgenic mouse carrying human immunoglobulin genes or by screening a human immunoglobulin gene library. A human antibody that is “derived from” a human germline immunoglobulin sequence can be identified as such by comparing the amino acid sequence of the human antibody to the amino acid sequence of human germline immunoglobulins. A selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 95%, or even at least 96%, 97%, 98%, or 99% identical in amino acid sequence to the amino acid sequence encoded by the germline immunoglobulin gene. Typically, a human antibody derived from a particular human germline sequence will display no more than 10 amino acid differences from the amino acid sequence encoded by the human germline immunoglobulin gene. In certain cases, the human antibody may display no more than 5, or even no more than 4, 3, 2, or 1 amino acid difference from the amino acid sequence encoded by the germline immunoglobulin gene.

A “human monoclonal antibody” refers to antibodies displaying a single binding specificity which have variable and constant regions derived from human germline immunoglobulin sequences. The term also intends recombinant human antibodies. Methods to making these antibodies are described herein.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the antibody, e.g., from a transfectoma, antibodies isolated from a recombinant, combinatorial human antibody library, and antibodies prepared, expressed, created or isolated by any other means that involve splicing of human immunoglobulin gene sequences to other DNA sequences. Such recombinant human antibodies have variable and constant regions derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. Methods to making these antibodies are described herein.

In terms of antibody structure, an immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, largely adopts a β-sheet conformation and the CDRs form loops which connect, and in some cases form part of, the β-sheet structure. Thus, framework regions act to form a scaffold that provides for positioning the CDRs in correct orientation by inter-chain, non-covalent interactions.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds HER2 or cathepsin B (also referred to herein as an anti-HER2 antibody or an anti-cathepsin B antibody) will have a specific V_{H} region and the V_{L} region sequence, and thus specific CDR sequences. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

As used herein, chimeric antibodies are antibodies whose light and heavy chain genes have been constructed, typically by genetic engineering, from antibody variable and constant region genes belonging to different species.

As used herein, the term “humanized antibody” or “humanized immunoglobulin” refers to a human/non-human chimeric antibody that contains a minimal sequence derived from non-human immunoglobulin. For the most part, humanized antibodies are human immunoglobulins (recipient antibody) in which residues from a variable region of the recipient are replaced by residues from a variable region of a non-human species (donor antibody) such as mouse, rat, rabbit, or non-human primate having the desired specificity, affinity and capacity. Humanized antibodies may comprise residues that are not found in the recipient antibody or in the donor antibody. The humanized antibody can optionally also comprise at least a portion of an immunoglobulin constant region (Fc), typically that of a human immunoglobulin, a non-human antibody containing one or more amino acids in a framework region, a constant region or a CDR, that have been substituted with a correspondingly positioned amino acid from a human antibody. In general, humanized antibodies are expected to produce a reduced immune response in a human host, as compared to a non-humanized version of the same antibody. The humanized antibodies may have conservative amino acid substitutions which have substantially no effect on antigen binding or other antibody functions. Conservative substitutions groupings include: glycine-alanine, valine-leucine-isoleucine, phenylalanine-tyrosine, lysine-arginine, alanine-valine, serine-threonine and asparagine-glutamine.

The terms “polyclonal antibody” or “polyclonal antibody composition” as used herein refer to a preparation of antibodies that are derived from different B-cell lines. They are a mixture of immunoglobulin molecules secreted against a specific antigen, each recognizing a different epitope.

As used herein, the term “antibody derivative”, comprises a full-length antibody or a fragment of an antibody, wherein one or more of the amino acids are chemically modified by alkylation, pegylation, acylation, ester formation or amide formation or the like, e.g., for linking the antibody to a second molecule. This includes, but is not limited to, pegylated antibodies, cysteine-pegylated antibodies, and variants thereof.

As used herein the term “linker sequence” relates to any amino acid sequence comprising from 1 to 10, or alternatively, 8 amino acids, or alternatively 6 amino acids, or alternatively 5 amino acids that may be repeated from 1 to 10, or alternatively to about 8, or alternatively to about 6, or alternatively about 5, or 4 or alternatively 3, or alternatively 2 times.

As used herein, the terms “treating,” “treatment,” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “composition” intends a carrier (liquid or solid support) and an active agent.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

A “biologically active agent” or an active agent disclosed herein intends one or more of an isolated or recombinant polypeptide, an isolated or recombinant polynucleotide, a vector, an isolated host cell, or an antibody, as well as compositions comprising one or more of same.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include intravenous administratin, oral administration, nasal administration, injection, and topical application.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The term “conjugated moiety” or “grafted” refers to a moiety that can be added to another by forming a covalent bond with a residue of chimeric polypeptide. The moiety may bond directly to a residue of the chimeric polypeptide or may form a covalent bond with a linker which in turn forms a covalent bond with a residue of the chimeric polypeptide.

The phrase “pharmaceutically acceptable polymer” refers to the group of compounds which can be conjugated to one or more polypeptides described here. It is contemplated that the conjugation of a polymer to the polypeptide is capable of extending the half-life of the polypeptide in vivo and in vitro. Non-limiting examples include polyethylene glycols, polyvinylpyrrolidones, polyvinylalcohols, cellulose derivatives, polyacrylates, polymethacrylates, sugars, polyols and mixtures thereof. The biological active agents can be conjugated to a pharmaceutically acceptable polymer for administration in accordance with the methods described herein.

A “gene delivery vehicle” is defined as any molecule that can carry inserted polynucleotides into a host cell. Examples of gene delivery vehicles are liposomes, micelles biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

A “yeast artificial chromosome” or “YAC” refers to a vector used to clone large DNA fragments (larger than 100 kb and up to 3000 kb). It is an artificially constructed chromosome and contains the telomeric, centromeric, and replication origin sequences needed for replication and preservation in yeast cells. Built using an initial circular plasmid, they are linearized by using restriction enzymes, and then DNA ligase can add a sequence or gene of interest within the linear molecule by the use of cohesive ends. Yeast expression vectors, such as YACs, Ylps (yeast integrating plasmid), and YEps (yeast episomal plasmid), are extremely useful as one can get eukaryotic protein products with posttranslational modifications as yeasts are themselves eukaryotic cells, however YACs have been found to be more unstable than BACs, producing chimeric effects.

A “viral vector” is defined as a recombinantly produced virus or viral particle that comprises a polynucleotide to be delivered into a host cell, either in vivo, ex vivo or in vitro. Examples of viral vectors include retroviral vectors, adenovirus vectors, adeno-associated virus vectors, alphavirus vectors and the like. Infectious tobacco mosaic virus (TMV)-based vectors can be used to manufacturer proteins and have been reported to express Griffithsin in tobacco leaves (O'Keefe et al. (2009) Proc. Nat. Acad. Sci. USA 106(15):6099-6104). Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, the term “label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. In one aspect, the label excludes naturally emitting molecules that are attached to a molecule in nature, e.g., a naturally fluorescing polynucleotide that is adjacent to a polynucleotide of interest in its native environment. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6^{th} ed). Examples of luminescent probes include, but are not limited to, aequorin and luciferases.

As used herein, the term “immunoconjugate” comprises an antibody or an antibody derivative associated with or linked to a second agent, such as a cytotoxic agent, a detectable agent, a radioactive agent, a targeting agent, a human antibody, a humanized antibody, a chimeric antibody, a synthetic antibody, a semisynthetic antibody, or a multispecific antibody.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, include, but are not limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

A “native” or “natural” antigen is a polypeptide, protein or a fragment which contains an epitope, which has been isolated from a natural biological source, and which can specifically bind to an antigen receptor, in particular a T cell antigen receptor (TCR), in a subject.

The terms “antigen” and “antigenic” refer to molecules with the capacity to be recognized by an antibody or otherwise act as a member of an antibody-ligand pair. Non-limiting examples include a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens “Specific binding” refers to the interaction of an antigen with the variable regions of immunoglobulin heavy and light chains. Antibody-antigen binding may occur in vivo or in vitro. The skilled artisan will understand that macromolecules, including proteins, nucleic acids, fatty acids, lipids, lipopolysaccharides and polysaccharides have the potential to act as an antigen. The skilled artisan will further understand that nucleic acids encoding a protein with the potential to act as an antibody ligand necessarily encode an antigen. The artisan will further understand that antigens are not limited to full-length molecules, but can also include partial molecules. The term “antigenic” is an adjectival reference to molecules having the properties of an antigen. The term encompasses substances which are immunogenic, i.e., immunogens, as well as substances which induce immunological unresponsiveness, or anergy, i.e., anergens.

As used herein, “solid phase support” or “solid support”, used interchangeably, is not limited to a specific type of support. Rather a large number of supports are available and are known to one of ordinary skill in the art. Solid phase supports include silica gels, resins, derivatized plastic films, glass beads, cotton, plastic beads, alumina gels. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories, etc.), POLYHIPE® resin (obtained from Aminotech, Canada), polyamide resin (obtained from Peninsula Laboratories), polystyrene resin grafted with polyethylene glycol (TentaGel®, Rapp Polymere, Tubingen, Germany) or polydimethylacrylamide resin (obtained from Milligen/Biosearch, Calif.).

An example of a solid phase support include glass, polystyrene, polypropylene, polyethylene, dextran, nylon, amylases, natural and modified celluloses, polyacrylamides, gabbros, and magnetite. The nature of the carrier can be either soluble to some extent or insoluble. The support material may have virtually any possible structural configuration so long as the coupled molecule is capable of binding to a polynucleotide, polypeptide or antibody. Thus, the support configuration may be spherical, as in a bead, or cylindrical, as in the inside surface of a test tube, or the external surface of a rod. Alternatively, the surface may be flat such as a sheet, test strip, etc. or alternatively polystyrene beads. Those skilled in the art will know many other suitable carriers for binding antibody or antigen, or will be able to ascertain the same by use of routine experimentation.

### MODES OF CARRYING OUT THE DISCLOSURE

This disclosure provides recombinant antibody fragments, polynucleotides encoding them as well as vectors and host cells containing the polynucleotides and their expression products. Also provided is a recombinant antibody with therapeutic activity.

In one aspect, provided herein is a recombinant polynucleotide encoding an antibody fragment, comprising, or alternatively consisting essentially of, or yet further consisting of, a polynucleotide encoding one, two or three inhibitory loop regions of stefin A incorporated into the Fab complementary determining regions (CDRs) light or heavy chains of an anti-HER2 antibody or an equivalent thereof. In one aspect the polynucleotide is a DNA, and in another aspect it is RNA. Non-limiting examples of anti-HER2 antibodies include a fully humanized anti-Her2 antibody described in EP2540745 A9, wherein the amino acid sequence of the heavy chain variable region is shown in SEQ ID NO. 6 and the amino acid sequence of light chain variable region is shown in SEQ ID NO. 8; the monoclonal antibodies against HER2 antigens disclosed in U.S. Pat. No. 8,722,362; and Herceptin (Trastuzumab).

The published (see genome.jp/dbget-bin/www bget?dr:D03257, last accessed on Mar. 28, 2016) sequences of the heavy and light chains are:

(Heavy chain) EVQLVESGGG LVQPGGSLRL SCAASGFNIK
DTYIHWVRQA PGKGLEWVAR IYPTNGYTRY ADSVKGRFTI
SADTSKNTAY LQMNSLRAED TAVYYCSRWGGDGFYAMDYW
GQGTLVTVSS ASTKGPSVFP LAPSSKSTSG GTAALGCLVK
DYFPEPVTVSWNSGALTSGV HTFPAVLQSS GLYSLSSVVT
VPSSSLGTQT YICNVNHKPS NTKVDKKVEPKSCDKTHTCP
PCPAPELLGG PSVFLFPPKP KDTLMISRTP EVTCVVVDVS
HEDPEVKFNWYVDGVEVHNA KTKPREEQYN STYRVVSVLT
VLHQDWLNGK EYKCKVSNKA LPAPIEKTIS KAKGQPREPQ
VYTLPPSREE MTKNQVSLTC LVKGFYPSDI AVEWESNGQP
ENNYKTTPPVLDSDGSFFLY SKLTVDKSRW QQGNVFSCSV
MHEALHNHYT QKSLSLSPG
(Disulfide bridge: 22-96; 147-203; 264-324; 370-
428, Dimer: 229; 232)
(Light chain) DIQMTQSPSS LSASVGDRVT ITCRASQDVN
TAVAWYQQKP GKAPKLLIY S ASF LYSGVPS RFSGSRSGTD
FTLTISSLQP EDFATYYCQQ HYTTPPTFGQGTKVEIKRTV
AAPSVFIFPP SDEQLKSGTA SVVCLLNNFY PREAKVQWKV
DNALQSGNSQESVTEQDSKD STYSLSSTLT LSKADYEKHK
VYACEVTHQG LSSPVTKSFN RGEC
(Disulfide bridge: 23-88; 134-194; H223-L214,
Dimer).

Also provided polynucleotides encoding equivalents of these polypeptides, as described herein. Non-limiting examples of polynucleotides and polypeptides are provided in the sequence listing immediately preceding the claims.

Recombinant Polynucleotides

In one aspect, this disclosure provides a recombinant polynucleotide encoding an antibody fragment, the fragment comprising, or consisting essentially of, or yet further consisting of: 1) a polynucleotide encoding one or more inhibitory loop regions of stefin A incorporated into a the Fab complementary determining regions (CDRs) heavy or light chains of an anti-HER2 antibody or an equivalent thereof; or 2) a polynucleotide encoding one or more inhibitory loops of stefin A substituted for the Fab loops of an anti-HER2 antibody or an equivalent thereof. While the HER-2 antibody may be a human antibody, it is not beyond the scope of this disclosure that equivalent antibodies include mammalian antibodies for use in veterinary or pre-clinical applications where appropriate. In one aspect, the anti-HER2 antibody is Herceptin or an equivalent thereof.

In one embodiment, the polynucleotide comprises, or consists essentially of, or yet further consists of: 1) a reversed stefin A loop 1 grafted between amino acid His91 and Thr93 in the light chain CDR3 of the anti-HER2 antibody; 2) the stefin A loop 2 inserted between the Asn30 and Thr31 in the anti-HER2 antibody light chain CDR1; and the stefin A loop 3 and linkers at the N- and C-termini between Tyr49 and Phe53 in the anti-HER2 antibody light chain CDR2, or an equivalent of each thereof.

In another aspect, the recombinant polynucleotide comprises, or consists essentially of, or yet further consists of: 1) a reversed stefin A loop 1 grafted between amino acid Gly101 and Gly103 in the heavy chain CDR3 of the anti-HER2 antibody; 2) the stefin A loop 2 inserted between the Asp31 and Thr32 in the anti-HER2 antibody heavy chain CDR1; and the stefin A loop 3 and linkers at the N- and C-termini between Ile51 and Gly56 in the anti-HER2 antibody heavy chain CDR2, or an equivalent of each thereof.

In another embodiment, the recombinant polynucleotide as described herein comprises, or consists essentially of, or yet further consists of: an inhibitory loop region of stefin A loop 3, with an optional linker, substituted into the CDR2 of the light chain between Tyr49 and Phe53 and the stefin loop 2 polynucleotide is reversed and inserted into a loop of the light chain between Arg66 and Phe71, or an equivalent of each thereof.

In one aspect, an inhibitory loop region of stefin A loop 2, with an optional coil-coil linkers at the N- and/or C-terminii, is substituted into the CDR1 of the light chain between Gln27 and Asn30 and the stefin A loop 3 polynucleotide, with an optionally coil-coil linkers at the N- and/or C-termini, and inserted into the CDR3 of between His91 and Thr94, or an equivalent of each thereof.

In a further aspect, two inhibitory loop regions are substituted for an anti-HER2 antibody CDR loops of the light chain, wherein stefin A loop2, with an optional coil-coil linkers at the N- and/or C-terminii, is substituted into the CDR1 of the light chain between Gln27 and Asn30, and the stefin A loop 3 polynucleotide, with an optional coil-coil linkers at the N- and/or C-termini, and inserted into the light chain CDR3 of between His91 and Thr94, or an equivalent of each thereof.

The recombinant polynucleotide can also comprise, or consists essentially of, or yet further consists of three inhibitory loop regions are substituted for an anti-HER2 antibody Fab CDR loops of the light chain, wherein stefin A loop2 is substituted into the CDR1 of the light chain between Asn30 and Thr31, and the stefin A loop 1 polynucleotide, in reverse, and inserted into the light chain CDR2 between Ser50 and Ala51, and stefin A loop is inserted into light chain CDR 3 between Gln90 and Thr97, or an equivalent of each thereof.

Alternatively, three inhibitory loops of stefin A are substituted for the Fab CDR loops of the light chain anti-HER2 antibody fragment, wherein stefin A loop 2 is inserted into the light chain CDR 1 between Asn30 and Thr31, and stefin A loop 3 with an optional linker at its N- and C-termini, and inserted into light chain CDR 2 between Tyr49 and Phe53, and stefin A loop 1 is reversed and inserted into the light chain CDR 3 between His 91 and Thr93, or an equivalent of each thereof.

Alternatively, wherein the stefin A loop 1 polynucleotide comprises, or consists essentially of, or yet further consists of:

CTGGGAGGTCCGATT;

and/or the stefin A loop 2 polynucleotide comprises, or consists essentially of, or yet further consists of:

GTCGTAGCGGGTACT;

and/or the Stefin A loop 3 polynucleotide comprises, or consists essentially of, or yet further consists of:

GGGGGCTCTAAAAGCCTCCCTGGGCAGAACGAAGATCTGAGCGGGGGT,

or an equivalent of each thereof.

In any of the above aspect the stefin A polynucleotide may further encode a linker of the amino acid sequence GGS.

Also provided herein is a recombinant stefin A-derived antibody fragment comprising an inhibitory loop region of stefin A incorporated into the Fab complementary determining regions (CDRs) light chains or heavy chains of an anti-HER2 antibody or an equivalent or each thereof. In one aspect, the anti-HER2 antibody is Herceptin or an equivalent thereof.

Also provided is a recombinant anti-HER2 antibody comprising a heavy or light chain antibody fragment described above and an N-terminal or C-terminal cathepsin B propeptide fusion polypeptide.

In one aspect, a recombinant polynucleotide encoding a propeptide-fused anti-HER2 antibody fragment comprises, or alternatively consists essentially of, or yet further consists of a polynucleotide encoding N-terminal or C-terminal propeptide cathepsin B fused to a polynucleotide encoding an anti-HER2 light or a heavy chain, e.g., Herceptin or an equivalent thereof.

In one aspect, the cathepsin B propeptide polynucleotide encodes a polypeptide comprising, or alternatively consisting essentially of, or yet further consisting of the amino acid sequence H₂N-RSRPSFHPLSDELVNYVNKRNTTWQAGHNFYNVDMSYLKRLCGTFLGGPKPPQRVMFTEDL-COOH.

In a number of the following sequences amino acids from Stefin A or propeptide (respectively) are bolded and GGS linkers are underlined.

Also provided is one or more isolated polynucleotides or polypeptides, comprising, or alternatively consisting essentially of, or yet further consisting of the sequences:

a) Stefin A-derived antibody Light Chain Amino Acid Sequence
DIQMTQSPSSLSASVGDRVTITCRASQDVNVVAGTTAVAWYQQKPGKAPKL
LIYGGSKSLPGQNEDLSGGFLYSGVPSRFSGSRSGTDFTLTISSLQPEDFATYYCQQH
LGGPITTPPTFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLS
SPVTKSFNRGEC;
or
b) Stefin A-derived antibody Light Chain DNA Sequence
gacatccagatgacccagtctccatcctccctgtctgcatctgtaggagacagagtcaccatcacttgccgggcaagtcag
gatgtgaatGTCGTAGCGGGTACTaccgcggtcgcatggtatcagcagaaaccagggaaagcccctaagctcctgat
ctatGGGGGCTCTAAAAGCCTCCCTGGGCAGAACGAAGATCTGAGCGGGGGTttat
gtatagtggggtcccatcaaggttcagtggcagtagatctgggacagatttcactctcaccatcagcagtctgcaacctgaagattttgc
aacttactactgtcaacagcatCTGGGAGGTCCGATTactacccctccgacgttcggccaaggtaccaagcttgagat
caaacgaactgtggctgcaccatctgtcttcatcttcccgccatctgatgagcagttgaaatctggaactgcctctgtcgtgtgcctgctg
aataacttctatcccagagaggccaaagtacagtggaaggtggataacgccctccaatcgggtaactcccaggagagtgtcacagag
caggacagcaaggacagcacctacagcctcagcagcaccctgacgctgagcaaagcagactacgagaaacacaaagtctacgcct
gcgaagtcacccatcagggcctgtcctcgcccgtcacaaagagatcaacaggggagagtgt;
or
c) Stefin A-derived antibody Heavy Chain Amino Acid Sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHT;
or
d) Stefin A-derived antibody Heavy Chain DNA Sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgagcccaaatcttgtgacaaaactcacaca;
or
e) Propeptide-antibody fusion Light Chain Amino Acid Sequence
RSRPSFHPLSDELVNYVNKRNTTWQAGHNFYNVDMSYLKRLCGTFLGGP
KPPQRVMFTEDLDIQMTQSPSSLSASVGDRVTITCRASQDVNTAVAWYQQKPGKA
PKLLIYSASFLYSGVPSRFSGSRSGTDFTLTISSLQPEDFATYYCQQHYTTPPTFGQGT
KLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC;
or
f) Propeptide-antibody fusion Light Chain DNA Sequence
CGTTCTCGTCCGTCTTTCCACCCGCTGTCTGACGAACTGGTTAACTAC
GTTAACAAACGTAACACCACCTGGCAGGCTGGTCACAACTTCTACAACGTTG
ACATGTCTTACCTGAAACGTCTGTGCGGTACCTTCCTGGGTGGTCCGAAACC
GCCGCAGCGTGTTATGTTCACCGAAGACCTGgacatccagatgacccagtctccatcctccctgtct
gcatctgtaggagacagagtcaccatcacttgccgggcaagtcaggatgtgaataccgcggtcgcatggtatcagcagaaaccagg
gaaagcccctaagctcctgatctattctgcatccttcttgtatagtggggtcccatcaaggttcagtggcagtagatctgggacagatttca
ctctcaccatcagcagtctgcaacctgaagattttgcaacttactactgtcaacagcattacactacccctccgacgttcggccaaggtac
caagcttgagatcaaacgaactgtggctgcaccatctgtcttcatcttcccgccatctgatgagcagttgaaatctggaactgcctctgtc
gtgtgcctgctgaataacttctatcccagagaggccaaagtacagtggaaggtggataacgccctccaatcgggtaactcccaggaga
gtgtcacagagcaggacagcaaggacagcacctacagcctcagcagcaccctgacgctgagcaaagcagactacgagaaacaca
aagtctacgcctgcgaagtcacccatcagggcctgtcctcgcccgtcacaaagagatcaacaggggagagtgt;
or
g) Propeptide-antibody fusion Heavy Chain Amino Acid Sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSP
GK;
or
h) Propeptide-antibody fusion Heavy Chain DNA Sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgaacccaaatcttgcgacaaaactcacacatgcccaccg
tgcccagcacctgaactcctggggggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactggctga
atggcaaggagtacaagtgcaaggtctccaacaaagccctcccagcccccatcgagaaaaccatctccaaagccaaagggcagcc
ccgagaaccacaggtgtacaccctgcccccatcccgggatgagctgaccaagaaccaggtcagcctgacctgcctggtcaaaggct
tctatcccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtgatgcatg
aggctctgcacaaccactacacgcagaagagcctctccctgtctccgggtaaa;
or
i) Gen1 Fab VL Amino Acid Sequence
DIQMTQSPSSLSASVGDRVTITCRASQDVNVVAGTTAVAWYQQKPGKAPKL
LIYGGSKSLPGQNEDLSGGFLYSGVPSRFSGSRSGTDFTLTISSLQPEDFATYYCQQH
LGGPITTPPTFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLS
SPVTKSFNRGEC;
j) Gen1 Fab VL DNA Sequence
gacatccagatgacccagtctccatcctccctgtctgcatctgtaggagacagagtcaccatcacttgccgggcaagtcag
gatgtgaatGTCGTAGCGGGTACTaccgcggtcgcatggtatcagcagaaaccagggaaagcccctaagctcctgat
ctatGGGGGCTCTAAAAGCCTCCCTGGGCAGAACGAAGATCTGAGCGGGGGTttctt
gtatagtggggtcccatcaaggttcagtggcagtagatctgggacagatttcactctcaccatcagcagtctgcaacctgaagattttgc
aacttactactgtcaacagcatCTGGGAGGTCCGATTactacccctccgacgttcggccaaggtaccaagcttgagat
caaacgaactgtggctgcaccatctgtcttcatcttcccgccatctgatgagcagttgaaatctggaactgcctctgtcgtgtgcctgctg
aataacttctatcccagagaggccaaagtacagtggaaggtggataacgccctccaatcgggtaactcccaggagagtgtcacagag
caggacagcaaggacagcacctacagcctcagcagcaccctgacgctgagcaaagcagactacgagaaacacaaagtctacgcct
gcgaagtcacccatcagggcctgtcctcgcccgtcacaaagagatcaacaggggagagtgt;
k) Gen1 Fab VH Amino Acid Sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHT;
l) Gen1 Fab VH DNA Sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgagcccaaatcttgtgacaaaactcacaca;
m) Propeptide N fusion VL Amino Acid Sequence
RSRPSFHPLSDELVNYVNKRNTTWQAGHNFYNVDMSYLKRLCGTFLGGP
KPPQRVMFTEDLDIQMTQSPSSLSASVGDRVTITCRASQDVNTAVAWYQQKPGKA
PKLLIYSASFLYSGVPSRFSGSRSGTDFTLTISSLQPEDFATYYCQQHYTTPPTFGQGT
KLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC;
n) Propeptide N fusion VL DNA Sequence
CGTTCTCGTCCGTCTTTCCACCCGCTGTCTGACGAACTGGTTAACTAC
GTTAACAAACGTAACACCACCTGGCAGGCTGGTCACAACTTCTACAACGTTG
ACATGTCTTACCTGAAACGTCTGTGCGGTACCTTCCTGGGTGGTCCGAAACC
GCCGCAGCGTGTTATGTTCACCGAAGACCTGgacatccagatgacccagtctccatcctccctgtct
gcatctgtaggagacagagtcaccatcacttgccgggcaagtcaggatgtgaataccgcggtcgcatggtatcagcagaaaccagg
gaaagcccctaagctcctgatctattctgcatccttcttgtatagtggggtcccatcaaggttcagtggcagtagatctgggacagatttca
ctctcaccatcagcagtctgcaacctgaagattttgcaacttactactgtcaacagcattacactacccctccgacgttcggccaaggtac
caagcttgagatcaaacgaactgtggctgcaccatctgtcttcatcttcccgccatctgatgagcagttgaaatctggaactgcctctgtc
gtgtgcctgctgaataacttctatcccagagaggccaaagtacagtggaaggtggataacgccctccaatcgggtaactcccaggaga
gtgtcacagagcaggacagcaaggacagcacctacagcctcagcagcaccctgacgctgagcaaagcagactacgagaaacaca
aagtctacgcctgcgaagtcacccatcagggcctgtcctcgcccgtcacaaagagatcaacaggggagagtgt;
o) Propeptide N fusion VH with FC Amino Acid Sequence (normal VH)
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSP
GK;
p) Propeptide N fusion VH with FC DNA Sequence (normal VH)
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgaacccaaatcttgcgacaaaactcacacatgcccaccg
tgcccagcacctgaactcctggggggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactggctga
atggcaaggagtacaagtgcaaggtctccaacaaagccctcccagcccccatcgagaaaaccatctccaaagccaaagggcagcc
ccgagaaccacaggtgtacaccctgcccccatcccgggatgagctgaccaagaaccaggtcagcctgacctgcctggtcaaaggct
tctatcccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtgatgcatg
aggctctgcacaaccactacacgcagaagagcctctccctgtctccgggtaaa;
q) Stefin A Loop X LC amino acid sequence
DIQMTQSPSSLSASVGDRVTITCRASQDVNTAVAWYQQKPGKAPKLLIYGGS
KSLPGQNEDLSGGFLYSGVPSRFSGSRKIYYNTGAVVQTKYQFTLTISSLQPEDFAT
YYCQQHYTTPPTFGQGTKLEIKRTVAAPSVFIFPPSDEQLKSGTASVVCLLNNFYPRE
AKVQWKVDNALQSGNSQESVTEQDSKDSTYSLSSTLTLSKADYEKHKVYACEVTH
QGLSSPVTKSFNRGEC;
r) Stefin A Loop X LC DNA sequence
gacatccagatgacccagtctccatcctccctgtctgcatctgtaggagacagagtcaccatcacttgccgggcaagtcag
gatgtgaataccgcggtcgcatggtatcagcagaaaccagggaaagcccctaagctcctgatctatGGGGGCTCTAAAA
GCCTCCCTGGGCAGAACGAAGATCTGAGCGGGGGTttcttgtatagtggggtcccatcaaggttcagt
ggcagtagaAAGATATATTACAATACTGGAGCGGTTGTGCAAACCAAGTATCAAttcactc
tcaccatcagcagtctgcaacctgaagattttgcaacttactactgtcaacagcattacactacccctccgacgttcggccaaggtacca
agcttgagatcaaacgaactgtggctgcaccatctgtcttcatcttcccgccatctgatgagcagttgaaatctggaactgcctctgtcgt
gtgcctgctgaataacttctatcccagagaggccaaagtacagtggaaggtggataacgccctccaatcgggtaactcccaggagagt
gtcacagagcaggacagcaaggacagcacctacagcctcagcagcaccctgacgctgagcaaagcagactacgagaaacacaaa
gtctacgcctgcgaagtcacccatcagggcctgtcctcgcccgtcacaaagagcttcaacaggggagagtgt;
s) Stefin A Loop X Heavy chain amino acid sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHT;
t) Stefin A Loop X Heavy chain DNA sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgagcccaaatcttgtgacaaaactcacaca;
u) Stefin A Coil-Coil LC amino acid sequence
DIQMTQSPSSLSASVGDRVTITCRASQGGSGAKLAALKAKLAALKGGGGSKT
QVVAGTNYYGGGGSELAALEAELAALEAGGSGNTAVAWYQQKPGKAPKLLIYSAS
FLYSGVPSRFSGSRSGTDFTLTISSLQPEDFATYYCQQHGGSGAKLAALKAKLAALK
GGGGSKVFKSLPGQNEDLVLTGGGGSELAALEAELAALEAGGSGTPPTFGQGTKLEI
KRTVAAPSVFIFPPSDEQLKSGTASVVCLLNNFYPREAKVQWKVDNALQSGNSQESV
TEQDSKDSTYSLSSTLTLSKADYEKHKVYACEVTHQGLSSPVTKSFNRGEC;
v) Stefin A Coil-Coil LC DNA sequence
gacatccagatgacccagtctccatcctccctgtctgcatctgtaggagacagagtcaccatcacttgccgggcaagtcag
gggggttccggcgcgaagttagcggcattaaaagctaaactcgcggctctcaaaggtggaggtggcagcAAAACCCAGG
TCGTAGCGGGTACTAACTACTACggtggcggcggatcggaacttgctgcgttggaagcggaacttgcggcgct
ggaagccggtgggagtggcaataccgcggtcgcatggtatcagcagaaaccagggaaagcccctaagctcctgatctattctgcatc
cttcttgtatagtggggtcccatcaaggttcagtggcagtagatctgggacagatttcactctcaccatcagcagtctgcaacctgaagat
tttgcaacttactactgtcaacagcatggcggctctggagcaaaattggctgcattaaaggcgaaactggcagcactgaaaggtggcg
gtggtagtAAAGTTTTCAAAAGCCTCCCTGGGCAGAACGAAGATCTGGTTCTGACCggc
ggaggcggatcggagctggcagccttggaagccgaactcgccgcacttgaagcgggaggtagcggcacccctccgacgttcggc
caaggtaccaagcttgagatcaaacgaactgtggctgcaccatctgtcttcatcttcccgccatctgatgagcagttgaaatctggaact
gcctctgtcgtgtgcctgctgaataacttctatcccagagaggccaaagtacagtggaaggtggataacgccctccaatcgggtaactc
ccaggagagtgtcacagagcaggacagcaaggacagcacctacagcctcagcagcaccctgacgctgagcaaagcagactacga
gaaacacaaagtctacgcctgcgaagtcacccatcagggcctgtcctcgcccgtcacaaagagcttcaacaggggagagtgt;
w) Stefin A Coil-Coil Heavy chain amino acid sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHT;
x) Stefin A Coil-Coil Heavy chain DNA sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgagcccaaatcttgtgacaaaactcacaca;
y) Stefin A CDR 2 3 Swap LC amino acid sequence
DIQMTQSPSSLSASVGDRVTITCRASQDVNVVAGTTAVAWYQQKPGKAPKL
LIYSLGGPIASFLYSGVPSRFSGSRSGTDFTLTISSLQPEDFATYYCQQKSLPGQNEDLT
FGQGTKLEIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSPVTKSFNR
GEC;
z) Stefin A CDR 2 3 Swap LC DNA sequence
gacatccagatgacccagtctccatcctccctgtctgcatctgtaggagacagagtcaccatcacttgccgggcaagtcag
gatgtgaatGTCGTAGCGGGTACTaccgcggtcgcatggtatcagcagaaaccagggaaagcccctaagctcctgatct
attctCTGGGAGGTCCGATTgcatccttcttgtatagtggggtcccatcaaggttcagtggcagtagatctgggacagattt
cactctcaccatcagcagtctgcaacctgaagatMgcaacttactactgtcaacagAAAAGCCTCCCTGGGCAGAA
CGAAGATCTGacgttcggccaaggtaccaagcttgagatcaaacgaactgtggctgcaccatctgtcttcatcttcccgccatc
tgatgagcagttgaaatctggaactgcctctgtcgtgtgcctgctgaataacttctatcccagagaggccaaagtacagtggaaggtgg
ataacgccctccaatcgggtaactcccaggagagtgtcacagagcaggacagcaaggacagcacctacagcctcagcagcaccct
gacgctgagcaaagcagactacgagaaacacaaagtctacgcctgcgaagtcacccatcagggcctgtcctcgcccgtcacaaaga
gcttcaacaggggagagtgt;
aa) Stefin A CDR 2 3 Swap Heavy chain amino acid sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHT;
bb) Stefin A CDR 2 3 Swap Heavy chain DNA sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgagcccaaatcttgtgacaaaactcacaca;
cc) Stefin A CDR 2 Ext LC amino acid sequence
DIQMTQSPSSLSASVGDRVTITCRASQDVNVVAGTTAVAWYQQKPGKAPKL
LIYGGSKVFKSLPGQNEDLVLTSGGFLYSGVPSRFSGSRSGTDFTLTISSLQPEDFATY
YCQQHLGGPITTPPTFGQGTKLEIKRTVAAPSVFIFPPSDEQLKSGTASVVCLLNNFYP
REAKVQWKVDNALQSGNSQESVTEQDSKDSTYSLSSTLTLSKADYEKHKVYACEVT
HQGLSSPVTKSFNRGEC;
dd) Stefin A CDR 2 Ext LC DNA sequence
gacatccagatgacccagtctccatcctccctgtctgcatctgtaggagacagagtcaccatcacttgccgggcaagtcag
gatgtgaatGTCGTAGCGGGTACTaccgcggtcgcatggtatcagcagaaaccagggaaagcccctaagctcctgatct
atGGGGGCTCTAAAGTTTTCAAAAGCCTCCCTGGGCAGAACGAAGATCTGGTTCTG
ACCAGCGGGGGTttatgtatagtggggtcccatcaaggttcagtggcagtagatctgggacagatttcactctcaccatcag
cagtctgcaacctgaagattttgcaacttactactgtcaacagcatCTGGGAGGTCCGATTactacccctccgacgttcgg
ccaaggtaccaagcttgagatcaaacgaactgtggctgcaccatctgtatcatcttcccgccatctgatgagcagttgaaatctggaac
tgcctctgtcgtgtgcctgctgaataacttctatcccagagaggccaaagtacagtggaaggtggataacgccctccaatcgggtaact
cccaggagagtgtcacagagcaggacagcaaggacagcacctacagcctcagcagcaccctgacgctgagcaaagcagactacg
agaaacacaaagtctacgcctgcgaagtcacccatcagggcctgtcctcgcccgtcacaaagagcttcaacaggggagagtgt;
ee) Stefin A CDR 2 Ext Heavy chain amino acid sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHT;
ff) Stefin A CDR 2 Ext Heavy chain DNA sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgagcccaaatcttgtgacaaaactcacaca;
gg) Propeptide fused to heavy chain N terminal LC amino acid sequence
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKLEIKRTVAA
PSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC;
hh) Propeptide fused to heavy chain N terminal LC DNA sequence
gacatccagatgacccagtctccatcctccctgtctgcatctgtaggagacagagtcaccatcacttgccgggcaagtcag
gatgtgaataccgcggtcgcatggtatcagcagaaaccagggaaagcccctaagctcctgatctattctgcatccttcttgtatagtggg
gtcccatcaaggttcagtggcagtagatctgggacagatttcactctcaccatcagcagtctgcaacctgaagattttgcaacttactact
gtcaacagcattacactacccctccgacgttcggccaaggtaccaagcttgagatcaaacgaactgtggctgcaccatctgtcttcatct
tcccgccatctgatgagcagttgaaatctggaactgcctctgtcgtgtgcctgctgaataacttctatcccagagaggccaaagtacagt
ggaaggtggataacgccctccaatcgggtaactcccaggagagtgtcacagagcaggacagcaaggacagcacctacagcctcag
cagcaccctgacgctgagcaaagcagactacgagaaacacaaagtctacgcctgcgaagtcacccatcagggcctgtcctcgcccg
tcacaaagagcttcaacaggggagagtgt;
ii) Propeptide fused to heavy chain N terminal HC amino acid sequence
RSRPSFHPLSDELVNYVNKRNTTWQAGHNFYNVDMSYLKRLCGTFLGGPKP
PQRVMFTEDLEVQLVESGGGLVQPGGSLRLSCAASGFNIKDTYIHWVRQAPGKGLE
WVARIYPTNGYTRYADSVKGRFTISADTSKNTAYLQMNSLRAEDTAVYYCSRWGG
DGFYAMDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYT
QKSLSLSPGK;
jj) Propeptide fused to heavy chain N terminal HC DNA sequence
CGTTCTCGTCCGTCTTTCCACCCGCTGTCTGACGAACTGGTTAACTACGTT
AACAAACGTAACACCACCTGGCAGGCTGGTCACAACTTCTACAACGTTGACATGT
CTTACCTGAAACGTCTGTGCGGTACCTTCCTGGGTGGTCCGAAACCGCCGCAGCG
TGTTATGTTCACCGAAGACCTGgaggtgcagctggtggagtctggaggaggcttggtccagcctggggggtcc
ctgagactctcctgtgcagcctctgggttcaatattaaggacacttacatccactgggtccgccaggctccagggaaggggctggagt
gggtcgcacgtatttatcctaccaatggttacacacgctacgcagactccgtgaagggccgattcaccatctccgcagacacttccaag
aacacggcgtatcttcaaatgaacagcctgagagccgaggacacggccgtgtattactgttcgagatggggcggtgacggcttctatg
ccatggactactggggccaaggaaccctggtcaccgtctcctcagcctccaccaagggcccatcggtcttccccctggcaccctcctc
caagagcacctctgggggcacagcggccctgggctgcctggtcaaggactacttccccgaaccggtgacggtgtcgtggaactcag
gcgccctgaccagcggcgtgcacaccttcccggctgtcctacagtcctcaggactctactccctcagcagcgtggtgactgtgccctct
agcagcttgggcacccagacctacatctgcaacgtgaatcacaagcccagcaacaccaaggtggacaagaaagttgaacccaaatc
ttgcgacaaaactcacacatgcccaccgtgcccagcacctgaactcctggggggaccgtcagtcttcctcttccccccaaaacccaag
gacaccctcatgatctcccggacccctgaggtcacatgcgtggtggtggacgtgagccacgaagaccctgaggtcaagttcaactgg
tacgtggacggcgtggaggtgcataatgccaagacaaagccgcgggaggagcagtacaacagcacgtaccgtgtggtcagcgtcc
tcaccgtcctgcaccaggactggctgaatggcaaggagtacaagtgcaaggtctccaacaaagccctcccagcccccatcgagaaa
accatctccaaagccaaagggcagccccgagaaccacaggtgtacaccctgcccccatcccgggatgagctgaccaagaaccagg
tcagcctgacctgcctggtcaaaggcttctatcccagcgacatcgccgtggagtgggagagcaatgggcagccggagaacaactac
aagaccacgcctcccgtgctggactccgacggctccttcttcctctacagcaagctcaccgtggacaagagcaggtggcagcaggg
gaacgtcttctcatgctccgtgatgcatgaggctctgcacaaccactacacgcagaagagcctctccctgtctccgggtaaa;
kk) Propeptide fused to heavy chain C terminal LC amino acid sequence
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKLEIKRTVAA
PSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC;
ll) Propeptide fused to heavy chain C terminal LC DNA sequence
gacatccagatgacccagtctccatcctccctgtctgcatctgtaggagacagagtcaccatcacttgccgggcaagtcag
gatgtgaataccgcggtcgcatggtatcagcagaaaccagggaaagcccctaagctcctgatctattctgcatccttcttgtatagtggg
gtcccatcaaggttcagtggcagtagatctgggacagatttcactctcaccatcagcagtctgcaacctgaagattttgcaacttactact
gtcaacagcattacactacccctccgacgttcggccaaggtaccaagcttgagatcaaacgaactgtggctgcaccatctgtatcatct
tcccgccatctgatgagcagttgaaatctggaactgcctctgtcgtgtgcctgctgaataacttctatcccagagaggccaaagtacagt
ggaaggtggataacgccctccaatcgggtaactcccaggagagtgtcacagagcaggacagcaaggacagcacctacagcctcag
cagcaccctgacgctgagcaaagcagactacgagaaacacaaagtctacgcctgcgaagtcacccatcagggcctgtcctcgcccg
tcacaaagagcttcaacaggggagagtgt;
mm) Propeptide fused to heavy chain C terminal HC amino acid sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQKSLSLSP
GKRSRPSFHPLSDELVNYVNKRNTTWQAGHNFYNVDMSYLKRLCGTFLGGPKPPQR
VMFTEDL;
nn) Propeptide fused to heavy chain C terminal HC DNA sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagatggggcggtgacggcttctatgccatggactactggggccaaggaaccct
ggtcaccgtctcctcagcctccaccaagggcccatcggtcttccccctggcaccctcctccaagagcacctctgggggcacagcggc
cctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatct
gcaacgtgaatcacaagcccagcaacaccaaggtggacaagaaagttgaacccaaatcttgcgacaaaactcacacatgcccaccg
tgcccagcacctgaactcctggggggaccgtcagtcttcctcttccccccaaaacccaaggacaccctcatgatctcccggacccctg
aggtcacatgcgtggtggtggacgtgagccacgaagaccctgaggtcaagttcaactggtacgtggacggcgtggaggtgcataat
gccaagacaaagccgcgggaggagcagtacaacagcacgtaccgtgtggtcagcgtcctcaccgtcctgcaccaggactggctga
atggcaaggagtacaagtgcaaggtctccaacaaagccctcccagcccccatcgagaaaaccatctccaaagccaaagggcagcc
ccgagaaccacaggtgtacaccctgcccccatcccgggatgagctgaccaagaaccaggtcagcctgacctgcctggtcaaaggct
tctatcccagcgacatcgccgtggagtgggagagcaatgggcagccggagaacaactacaagaccacgcctcccgtgctggactcc
gacggctccttcttcctctacagcaagctcaccgtggacaagagcaggtggcagcaggggaacgtcttctcatgctccgtgatgcatg
aggctctgcacaaccactacacgcagaagagcctctccctgtctccgggtaaaCGTTCTCGTCCGTCTTTCCACC
CGCTGTCTGACGAACTGGTTAACTACGTTAACAAACGTAACACCACCTGGCAGGC
TGGTCACAACTTCTACAACGTTGACATGTCTTACCTGAAACGTCTGTGCGGTACC
TTCCTGGGTGGTCCGAAACCGCCGCAGCGTGTTATGTTCACCGAAGACCTG;
oo) Propeptide fused to heavy chain CDR 3 Xa LC amino acid sequence
DIQMTQSPSSLSASVGDRVTITCRASQDVNTAVAWYQQKPGKAPKLLIYSASF
LYSGVPSRFSGSRSGTDFTLTISSLQPEDFATYYCQQHYTTPPTFGQGTKLEIKRTVAA
PSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSFNRGEC;
pp) Propeptide fused to heavy chain CDR 3 Xa LC DNA sequence
gacatccagatgacccagtctccatcctccctgtctgcatctgtaggagacagagtcaccatcacttgccgggcaagtcag
gatgtgaataccgcggtcgcatggtatcagcagaaaccagggaaagcccctaagctcctgatctattctgcatccttcttgtatagtggg
gtcccatcaaggttcagtggcagtagatctgggacagatttcactctcaccatcagcagtctgcaacctgaagattttgcaacttactact
gtcaacagcattacactacccctccgacgttcggccaaggtaccaagcttgagatcaaacgaactgtggctgcaccatctgtcttcatct
tcccgccatctgatgagcagttgaaatctggaactgcctctgtcgtgtgcctgctgaataacttctatcccagagaggccaaagtacagt
ggaaggtggataacgccctccaatcgggtaactcccaggagagtgtcacagagcaggacagcaaggacagcacctacagcctcag
cagcaccctgacgctgagcaaagcagactacgagaaacacaaagtctacgcctgcgaagtcacccatcagggcctgtcctcgcccg
tcacaaagagcttcaacaggggagagtgt;
qq) Propeptide fused to heavy chain CDR 3 Xa HC amino acid sequence
EVQLVESGGGLVQPGGSLRLSCAASGFNIKDTYIHWVRQAPGKGLEWVARIY
PTNGYTRYADSVKGRFTISADTSKNTAYLQMNSLRAEDTAVYYCSRGGSGAKLAAL
KAKLAALKCGGGGSIEGRRSRPSFHPLSDELVNYVNKRNTTWQAGHNFYNVDMSY
LKRLCGTFLGGPKPPQRVMFTEDLGGGGSCELAALEAELAALEAGGSGDYWGQGTL
VTVSSASTKGPSVFPLAPSSKSTSGGTAALGCLVKDYFPEPVTVSWNSGALTSGVHTF
PAVLQSSGLYSLSSVVTVPSSSLGTQTYICNVNHKPSNTKVDKKVEPKSCDKTHTCPP
CPAPPVAGPSVFLFPPKPKDTLMISRTPEVTCVVVDVSHEDPEVKFNWYVDGVEVHN
AKTKPREEQYNSTYRVVSVLTVLHQDWLNGKEYKCKVSNKGLPSSIEKTISKAKGQP
REPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK;
rr) Propeptide fused to heavy chain CDR 3 Xa HC DNA sequence
gaggtgcagctggtggagtctggaggaggcttggtccagcctggggggtccctgagactctcctgtgcagcctctgggtt
caatattaaggacacttacatccactgggtccgccaggctccagggaaggggctggagtgggtcgcacgtatttatcctaccaatggtt
acacacgctacgcagactccgtgaagggccgattcaccatctccgcagacacttccaagaacacggcgtatcttcaaatgaacagcct
gagagccgaggacacggccgtgtattactgttcgagaGGCGGAAGCGGAGCAAAGCTCGCCGCACTG
AAAGCCAAGCTGGCCGCTCTGAAGTGCGGGGGTGGCGGAAGCatcgaaggtcgtCGTTC
TCGTCCGTCTTTCCACCCGCTGTCTGACGAACTGGTTAACTACGTTAACAAACGT
AACACCACCTGGCAGGCTGGTCACAACTTCTACAACGTTGACATGTCTTACCTGA
AACGTCTGTGCGGTACCTTCCTGGGTGGTCCGAAACCGCCGCAGCGTGTTATGTT
CACCGAAGACCTGGGCGGAGGTGGGAGTTGCGAACTGGCCGCACTGGAAGCTGA
GCTGGCTGCCCTCGAAGCTGGAGGCTCTGGAgactactggggccaaggaaccctggtcaccgtctcctc
agcctccaccaagggcccatcggtatccccctggcaccctcctccaagagcacctctgggggcacagcggccctgggctgcctgg
tcaaggactacttccccgaaccggtgacggtgtcgtggaactcaggcgccctgaccagcggcgtgcacaccttcccggctgtcctac
agtcctcaggactctactccctcagcagcgtggtgactgtgccctctagcagcttgggcacccagacctacatctgcaacgtgaatcac
aagcccagcaacaccaaggtggacaagaaagttgaacccaaatcttgcgacaaaactcacacatgcccaccgtgcccagcacctC
CaGtcGCcggaccgtcagtcttcctcttcccTccaaaacccaaggacaccctcatgatctcccggacccctgaggtcacatgcgtg
gtggtggacgtgagccacgaagaccctgaggtcaagttcaactggtacgtggacggcgtggaggtgcataatgccaagacaaagcc
gcgggaggagcagtacaacagcacgtaccgtgtggtcagcgtcctcaccgtcctgcaccaggactggctgaatggcaaggagtaca
agtgcaaggtctccaacaaagGcctcccaAGcTccatcgagaaaaccatctccaaagccaaagggcagccccgagaaccacag
gtgtacaccctgccTccatcccgggatgagctgaccaagaaccaggtcagcctgacctgcctggtcaaaggcttctatcccagcgac
atcgccgtggagtgggagagcaatgggcagccggagaacaactacaagaccacgcctcccgtgctggactccgacggctccttctt
cctctacagcaagctcaccgtggacaagagcaggtggcagcaggggaacgtcttctcatgctccgtgatgcatgaggctctgcacaa
ccactacacgcagaagagcctctccctgtctccgggtaaa;
or
ss) an equivalent of each thereof

In a further aspect, the recombinant polynucleotide is operatively linked to the appropriate regulatory polynucleotides for expression in eukaryotic or prokaryotic cells. The recombinant polynucleotides can be further inserted into an expression vector for expression in a prokaryotic or eukaryotic cell. In one aspect, the vector is appropriate for expression in a bacterial cell, e.g. a plasmid vector and the host cell can be a prokaryotic cell such as a bacterial cell. In another aspect the vector is chosen for expression in a eukaryotic cell, e.g., a viral vector and the cell is a eukaryotic cell, e.g., a mammalian cell, e.g., a murine, a rat, a bovine, an equine, a canine, a feline, or a human cell for recombinant expression. The host cell systems can be used to recombinantly express the polynucleotide by growing the host cell under conditions to express the recombinant polynucleotide and in a further aspect, isolating the expression product from the host cell. Accordingly, this disclosure also provides a recombinant polypeptide isolated from this culture system.

Also provided is a vector comprising, or alternatively consisting essentially of, or yet further consisting of, one or more recombinant polynucleotide(s) disclosed above, that is optionally operatively linked to a detectable label and/or regulatory elements for expression of the polynucleotide. In one aspect, the vector is a vector for expression of the polynucleotide in a prokaryotic or eukaryotic host cell. Yet further provided is a host cell comprising, or alternatively consisting essentially of, or yet further consisting of, the recombinant polynucleotide or the vector described above. The host cells can be used for expression of the polynucleotides by growing the host cell under conditions for expression of the polynucleotide that in one aspect is isolated from the cell or cell culture media. Any of the above compositions can be combined with a carrier as described herein for use in commercial or clinical applications, e.g., diagnostically or therapeutically.

Expression vectors containing these nucleic acids are useful to obtain host vector systems to produce proteins and polypeptides. It is implied that these expression vectors must be replicable in the host organisms either as episomes or as an integral part of the chromosomal DNA. Non-limiting examples of suitable expression vectors include plasmids, yeast vectors, viral vectors and liposomes. Adenoviral vectors are particularly useful for introducing genes into tissues in vivo because of their high levels of expression and efficient transformation of cells both in vitro and in vivo. When a nucleic acid is inserted into a suitable host cell, e.g., a prokaryotic or a eukaryotic cell and the host cell replicates, the protein can be recombinantly produced. Suitable host cells will depend on the vector and can include mammalian cells, animal cells, human cells, simian cells, insect cells, yeast cells, and bacterial cells constructed using known methods. See, Sambrook et al. (1989) supra. In addition to the use of viral vector for insertion of exogenous nucleic acid into cells, the nucleic acid can be inserted into the host cell by methods known in the art such as transformation for bacterial cells; transfection using calcium phosphate precipitation for mammalian cells; or DEAE-dextran; electroporation; or microinjection. See, Sambrook et al. (1989) supra, for methodology. Thus, this disclosure also provides a host cell, e.g., a mammalian cell, an animal cell (rat or mouse), a human cell, or a prokaryotic cell such as a bacterial cell, containing a polynucleotide encoding a protein or polypeptide or antibody.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

When the vectors are used for gene therapy in vivo or ex vivo, a pharmaceutically acceptable vector, such as a replication-incompetent retroviral or adenoviral vector, are exemplary (but non-limiting) and may be of particular use. Pharmaceutically acceptable vectors containing the nucleic acids disclosed herein can be further modified for transient or stable expression of the inserted polynucleotide. As used herein, the term “pharmaceutically acceptable vector” includes, but is not limited to, a vector or delivery vehicle having the ability to selectively target and introduce the nucleic acid into dividing cells. An example of such a vector is a “replication-incompetent” vector defined by its inability to produce viral proteins, precluding spread of the vector in the infected host cell. An example of a replication-incompetent retroviral vector is LNL6 (Miller et al. (1989) BioTechniques 7:980-990). The methodology of using replication-incompetent retroviruses for retroviral-mediated gene transfer of gene markers has been established. (Bordignon (1989) PNAS USA 86:8912-8952; Culver (1991) PNAS USA 88:3155; and Rill (1991) Blood 79(10):2694-2700).

This disclosure also provides genetically modified cells that contain and/or express the polynucleotides disclosed herein. The genetically modified cells can be produced by insertion of upstream regulatory sequences such as promoters or gene activators (see, U.S. Pat. No. 5,733,761).

The polynucleotides can be conjugated to a detectable marker, e.g., an enzymatic label or a radioisotope for detection of nucleic acid and/or expression of the gene in a cell. A wide variety of appropriate detectable markers are known in the art, including fluorescent, radioactive, enzymatic or other ligands, such as avidin/biotin, which are capable of giving a detectable signal. In one aspect, one will likely desire to employ a fluorescent label or an enzyme tag, such as urease, alkaline phosphatase or peroxidase, instead of radioactive or other environmentally undesirable reagents. In the case of enzyme tags, calorimetric indicator substrates can be employed to provide a means visible to the human eye or spectrophotometrically, to identify specific hybridization with complementary nucleic acid-containing samples. Thus, this disclosure further provides a method for detecting a single-stranded polynucleotide or its complement, by contacting target single-stranded polynucleotide with a labeled, single-stranded polynucleotide (a probe) which is a portion of the polynucleotide disclosed herein under conditions permitting hybridization (optionally moderately stringent hybridization conditions) of complementary single-stranded polynucleotides, or optionally, under highly stringent hybridization conditions. Hybridized polynucleotide pairs are separated from un-hybridized, single-stranded polynucleotides. The hybridized polynucleotide pairs are detected using methods known to those of skill in the art and set forth, for example, in Sambrook et al. (1989) supra.

The polynucleotide embodied in this disclosure can be obtained using chemical synthesis, recombinant cloning methods, PCR, or any combination thereof. Methods of chemical polynucleotide synthesis are known in the art and need not be described in detail herein. One of skill in the art can use the sequence data provided herein to obtain a desired polynucleotide by employing a DNA synthesizer or ordering from a commercial service.

The polynucleotides disclosed herein can be isolated or replicated using PCR. The PCR technology is the subject matter of U.S. Pat. Nos. 4,683,195; 4,800,159; 4,754,065; and 4,683,202 and described in PCR: The Polymerase Chain Reaction (Mullis et al. eds., Birkhauser Press, Boston (1994)) or MacPherson et al. (1991) and (1995) supra, and references cited therein. Alternatively, one of skill in the art can use the sequences provided herein and a commercial DNA synthesizer to replicate the DNA. Accordingly, this disclosure also provides a process for obtaining the polynucleotides disclosed herein by providing the linear sequence of the polynucleotide, nucleotides, appropriate primer molecules, chemicals such as enzymes and instructions for their replication and chemically replicating or linking the nucleotides in the proper orientation to obtain the polynucleotides. In a separate embodiment, these polynucleotides are further isolated. Still further, one of skill in the art can insert the poly-nucleotide into a suitable replication vector and insert the vector into a suitable host cell (prokaryotic or eukaryotic) for replication and amplification. The DNA so amplified can be isolated from the cell by methods known to those of skill in the art. A process for obtaining polynucleotides by this method is further provided herein as well as the polynucleotides so obtained.

RNA can be obtained by first inserting a DNA polynucleotide into a suitable host cell. The DNA can be delivered by any appropriate method, e.g., by the use of an appropriate gene delivery vehicle (e.g., liposome, plasmid or vector) or by electroporation. When the cell replicates and the DNA is transcribed into RNA; the RNA can then be isolated using methods known to those of skill in the art, for example, as set forth in Sambrook et al. (1989) supra. For instance, mRNA can be isolated using various lytic enzymes or chemical solutions according to the procedures set forth in Sambrook et al. (1989) supra, or extracted by nucleic-acid-binding resins following the accompanying instructions provided by manufactures.

Polynucleotides exhibiting sequence complementarity or homology to a polynucleotide disclosed herein are useful as hybridization probes or as an equivalent of the specific polynucleotides identified herein. Since the full coding sequence of the transcript is known, any portion of this sequence or homologous sequences can be used in the methods disclosed herein.

It is known in the art that a “perfectly matched” probe is not needed for a specific hybridization. Minor changes in probe sequence achieved by substitution, deletion or insertion of a small number of bases do not affect the hybridization specificity. In general, as much as 20% base-pair mismatch (when optimally aligned) can be tolerated. In some embodiments, a probe useful for detecting the aforementioned mRNA is at least about 80% identical to the homologous region. In some embodiments, the probe is 85% identical to the corresponding gene sequence after alignment of the homologous region; in some embodiments, it exhibits 90% identity.

These probes can be used in radioassays (e.g., Southern and Northern blot analysis) to detect, prognose, diagnose or monitor various cells or tissues containing these cells. The probes also can be attached to a solid support or an array such as a chip for use in high throughput screening assays for the detection of expression of the gene corresponding a polynucleotide disclosed herein. Accordingly, this disclosure also provides a probe comprising or corresponding to a polynucleotide disclosed herein, or its equivalent, or its complement, or a fragment thereof, attached to a solid support for use in high throughput screens.

The total size of fragment, as well as the size of the complementary stretches, will depend on the intended use or application of the particular nucleic acid segment. Smaller fragments will generally find use in hybridization embodiments, wherein the length of the complementary region may be varied, such as between at least 5 to 10 to about 100 nucleotides, or even full length according to the complementary sequences one wishes to detect.

Nucleotide probes having complementary sequences over stretches greater than 5 to 10 nucleotides in length are generally well suited, so as to increase stability and selectivity of the hybrid, and thereby improving the specificity of particular hybrid molecules obtained. In certain embodiments, one can design polynucleotides having gene-complementary stretches of 10 or more or more than 50 nucleotides in length, or even longer where desired. Such fragments may be readily prepared by, for example, directly synthesizing the fragment by chemical means, by application of nucleic acid reproduction technology, such as the PCR technology with two priming oligonucleotides as described in U.S. Pat. No. 4,603,102 or by introducing selected sequences into recombinant vectors for recombinant production. In one aspect, a probe is about 50-75 or more alternatively, 50-100, nucleotides in length.

The polynucleotides of the present disclosure can serve as primers for the detection of genes or gene transcripts that are expressed in cells described herein as well as to prepare the expression products. In the former context, amplification means any method employing a primer-dependent polymerase capable of replicating a target sequence with reasonable fidelity. Amplification may be carried out by natural or recombinant DNA-polymerases such as T7 DNA polymerase, Klenow fragment of E. coli DNA polymerase, and reverse transcriptase. For illustration purposes only, a primer is the same length as that identified for probes.

One method to amplify polynucleotides is PCR and kits for PCR amplification are commercially available. After amplification, the resulting DNA fragments can be detected by any appropriate method known in the art, e.g., by agarose gel electrophoresis followed by visualization with ethidium bromide staining and ultraviolet illumination.

Methods for administering an effective amount of a gene delivery vector or vehicle to a cell have been developed and are known to those skilled in the art and described herein. Methods for detecting gene expression in a cell are known in the art and include techniques such as in hybridization to DNA microarrays, in situ hybridization, PCR, RNase protection assays and Northern blot analysis. Such methods are useful to detect and quantify expression of the gene in a cell. Alternatively expression of the encoded polypeptide can be detected by various methods. In particular it is useful to prepare polyclonal or monoclonal antibodies that are specifically reactive with the target polypeptide. Such antibodies are useful for visualizing cells that express the polypeptide using techniques such as immunohistology, ELISA, and Western blotting. These techniques can be used to determine expression level of the expressed polynucleotide.

Antibodies and Derivatives Thereof

In another aspect, this disclosure provides a recombinant cathepsin B antibody that inhibits proteolytic activity of human cathepsin B. In a further aspect, the antibody inhibits the proteolytic activity of human cathepsin B in a dose-dependent fashion. The antibody comprises, or alternatively consists essentially of, or yet further comprises the recombinant light and heavy chains as described herein. Non-limiting examples of the polypeptide and polynucleotide sequences of the antibody heavy and light chains are provided herein.

Also provided herein is a recombinant stefin A-derived antibody or antibody fragment comprising, or alternatively consisting essentially of, or yet further consisting of, one or more of the three inhibitory loop region polypeptides of stefin A incorporated into the Fab complementary determining regions (CDRs) light chains or heavy of an anti-HER2 antibody or an equivalent or each thereof. Non-limiting examples of anti-HER2 antibodies include a fully humanized anti-her2 antibody described in EP2540745 A9, wherein the amino acid sequence of the heavy chain variable region is shown in SEQ ID NO. 6 and the amino acid sequence of light chain variable region is shown in SEQ ID NO. 8; the monoclonal antibodies against HER2 antigens disclosed in U.S. Pat. No. 8,722,362; and Herceptin (Trastuzumab).

The published (see genome.jp/dbget-bin/www_bget?dr:D03257, last accessed on Mar. 28, 2016) sequences of the heavy and light chains are:

(Heavy chain) EVQLVESGGG LVQPGGSLRL SCAASGFNIK
DTYIHWVRQA PGKGLEWVAR IYPTNGYTRY ADSVKGRFTI
SADTSKNTAY LQMNSLRAEDTAVYYCSRWGGDGFYAMDYW
GQGTLVTVSS ASTKGPSVFP LAPSSKSTSG GTAALGCLVK
DYFPEPVTVSWNSGALTSGV HTFPAVLQSS GLYSLSSVVT
VPSSSLGTQT YICNVNHKPS NTKVDKKVEPKSCDKTHTCP
PCPAPELLGG PSVFLFPPKP KDTLMISRTP EVTCVVVDVS
HEDPEVKFNW YVDGVEVHNA KTKPREEQYN STYRVVSVLT
VLHQDWLNGK EYKCKVSNKA LPAPIEKTISKAKGQPREPQ
VYTLPPSREE MTKNQVSLTC LVKGFYPSDI AVEWESNGQP
ENNYKTTPPVLDSDGSFFLY SKLTVDKSRW QQGNVFSCSV
MHEALHNHYT QKSLSLSPG
(Disulfide bridge: 22-96; 147-203; 264-324; 370-
428, Dimer: 229; 232)
(Light chain) DIQMTQSPSS LSASVGDRVT ITCRASQDVN
TAVAWYQQKP GKAPKLLIYS ASFLYSGVPS RFSGSRSGTD
FTLTISSLQP EDFATYYCQQ HYTTPPTFGQGTKVEIKRTV
AAPSVFIFPP SDEQLKSGTA SVVCLLNNFY PREAKVQWKV
DNALQSGNSQESVTEQDSKD STYSLSSTLT LSKADYEKHK
VYACEVTHQG LSSPVTKSFN RGEC
(Disulfide bridge: 23-88; 134-194; H223-L214,
Dimer).

In some aspects, the antibody or antigen binding fragment provided herein is encoded for by one or more of the recombinant polynucleotides disclosed herein above. In some aspects, the antibody or antigen binding fragment provided herein comprises one or more of the polypeptide sequences disclosed herein above.

In another aspect of the present technology, the antibody or antigen binding fragment includes one or more of the following characteristics:

(a) the light chain immunoglobulin variable domain sequence comprises one or more CDRs that are at least 85% identical to a CDR of a light chain variable domain of any of the disclosed light chain sequences;

(b) the heavy chain immunoglobulin variable domain sequence comprises one or more CDRs that are at least 85% identical to a CDR of a heavy chain variable domain of any of the disclosed heavy chain sequences;

(c) the light chain immunoglobulin variable domain sequence is at least 85% identical to a light chain variable domain of any of the disclosed light chain sequences;

(d) the HC immunoglobulin variable domain sequence is at least 85% identical to a heavy chain variable domain of any of the disclosed light chain sequences; and/or

(e) the antibody binds an epitope that overlaps with an epitope or conformational epitope bound by any of the disclosed sequences.

In some aspects of the antibody or antigen binding fragment provided herein, the antibody or antigen binding fragment binds cathepsin B with a dissociation or inhibition constant (K_{D} or Kᵢ) of less than 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, or 10⁻¹² M. In some of the aspects of the antibodies provided herein, the antigen binding site specifically binds to cathepsin B.

In some aspects of the antibody or antigen binding fragment provided herein, the antibody or antigen binding fragment is soluble Fab.

In some aspects of the antibody or antigen binding fragment provided herein, the HC and LC variable domain sequences are components of the same polypeptide chain. In some of the aspects of the antibodies provided herein, the HC and LC variable domain sequences are components of different polypeptide chains.

In some aspects of the antibody or antigen binding fragment provided herein, the antibody or antigen binding fragment is a full-length antibody.

In some aspects of the antibody or antigen binding fragment provided herein, the antibody or antigen binding fragment is a monoclonal antibody.

In some aspects of the antibody or antigen binding fragment provided herein, the antibody or antigen binding fragment is chimeric or humanized.

In some aspects of the antibody or antigen binding fragment provided herein, the antibody or antigen binding fragment is selected from the group consisting of Fab, F(ab)′2, Fab′, scFᵥ, and Fᵥ.

In some aspects of the antibody or antigen binding fragment provided herein, the antibody or antigen binding fragment comprises an Fc domain. In some aspects of the antibody or antigen binding fragment provided herein, the antibody or antigen binding fragment is a non-human animal such as a mouse, rat, sheep, bovine, canine, feline or rabbit antibody. In some aspects of the antibody or antigen binding fragment provided herein, the antibody or antigen binding fragment is a human or humanized antibody or is non-immunogenic in a human.

In some aspects of the antibody or antigen binding fragment provided herein, the antibody or antigen binding fragment comprises a human antibody framework region.

In other aspects, one or more amino acid residues in a CDR of the antibodies provided herein are substituted with another amino acid. The substitution may be “conservative” in the sense of being a substitution within the same family of amino acids. The naturally occurring amino acids may be divided into the following four families and conservative substitutions will take place within those families.

1) Amino acids with basic side chains: lysine, arginine, histidine.

2) Amino acids with acidic side chains: aspartic acid, glutamic acid

3) Amino acids with uncharged polar side chains: asparagine, glutamine, serine, threonine, tyrosine.

4) Amino acids with nonpolar side chains: glycine, alanine, valine, leucine, isoleucine, proline, phenylalanine, methionine, tryptophan, cysteine.

In another aspect, one or more amino acid residues are added to or deleted from one or more CDRs of an antibody. Such additions or deletions occur at the N or C termini of the CDR or at a position within the CDR. By varying the amino acid sequence of the CDRs of an antibody by addition, deletion or substitution of amino acids, various effects such as increased binding affinity for the target antigen may be obtained. It is to be appreciated that antibodies of the present disclosure comprising such varied CDR sequences still bind with similar specificity and sensitivity profiles as the disclosed antibodies. This may be tested by way of the binding assays disclosed herein above with respect to equivalents.

The variable region of the antibodies of the present disclosure can be modified by mutating amino acid residues within the VH and/or VL CDR 1, CDR 2 and/or CDR 3 regions to improve one or more binding properties (e.g., affinity) of the antibody. Mutations may be introduced by site-directed mutagenesis or PCR-mediated mutagenesis and the effect on antibody binding, or other functional property of interest, can be evaluated in appropriate in vitro or in vivo assays. In certain embodiments, conservative modifications are introduced and typically no more than one, two, three, four or five residues within a CDR region are altered. The mutations may be amino acid substitutions, additions or deletions.

In some aspects disclosed herein, it will be useful to detectably or therapeutically label the antibody. Suitable labels are described supra. Methods for conjugating antibodies to these agents are known in the art. For the purpose of illustration only, antibodies can be labeled with a detectable moiety such as a radioactive atom, a chromophore, a fluorophore, or the like. Such labeled antibodies can be used for diagnostic techniques, either in vivo, or in an isolated test sample.

The coupling of antibodies to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten antibodies. See, Harlow and Lane (1988) supra.

Framework modifications can be made to the antibodies to decrease immunogenicity, for example, by “backmutating” one or more framework residues to the corresponding germline sequence.

The antibodies or antigen binding fragments disclosed herein may also comprise one or more of the variable regions disclosed herein and suitable constant domains, e.g. the non-limiting exemplary domains provided herein below:

Human IgD constant region, Uniprot: P01880:
APTKAPDVFPIISGCRHPKDNSPVVLACLITGYHPTSVTVTWYMGTQSQPQRTFPEIQ
RRDSYYMTSSQLSTPLQQWRQGEYKCVVQHTASKSKKEIFRWPESPKAQASSVPTA
QPQAEGSLAKATTAPATTRNTGRGGEEKKKEKEKEEQEERETKTPECPSHTQPLGVY
LLTPAVQDLWLRDKATFTCFVVGSDLKDAHLTWEVAGKVPTGGVEEGLLERHSNG
SQSQHSRLTLPRSLWNAGTSVTCTLNHPSLPPQRLMALREPAAQAPVKLSLNLLASS
DPPEAASWLLCEVSGFSPPNILLMWLEDQREVNTSGFAPARPPPQPGSTTFWAWSVL
RVPAPPSPQPATYTCVVSHEDSRTLLNASRSLEVSYVTDHGPMK.
Human IgG1 constant region, Uniprot: P01857:
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
ELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK.
Human IgG2 constant region, Uniprot: P01859:
ASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVY
TLPPSREEMTKNQVSLTCLVKGFYPSDISVEWESNGQPENNYKTTPPMLDSDGSFFL
YSKLTVDKSRWQQGNVFSCSVMHEALHNHYTQKSLSLSPGK.
Human IgG3 constant region, Uniprot: P01860:
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTPLGDTTHTCPRC
PEPKSCDTPPPCPRCPEPKSCDTPPPCPRCPEPKSCDTPPPCPRCPAPELLGGPSVFLFPP
KPKDTLMISRTPEVTCVVVDVSHEDPEVQFKWYVDGVEVHNAKTKPREEQYNSTFR
VVSVLTVLHQDWLNGKEYKCKVSNKALPAPIEKTISKTKGQPREPQVYTLPPSREEM
TKNQVSLTCLVKGFYPSDIAVEWESSGQPENNYNTTPPMLDSDGSFFLYSKLTVDKS
RWQQGNIFSCSVMHEALHNRFTQKSLSLSPGK.
Human IgM constant region, Uniprot: P01871:
GSASAPTLFPLVSCENSPSDTSSVAVGCLAQDFLPDSITLSWKYKNNSDISSTRGFPSV
LRGGKYAATSQVLLPSKDVMQGTDEHVVCKVQHPNGNKEKNVPLPVIAELPPKVSV
FVPPRDGFFGNPRKSKLICQATGFSPRQIQVSWLREGKQVGSGVTTDQVQAEAKESG
PTTYKVTSTLTIKESDWLGQSMFTCRVDHRGLTFQQNASSMCVPDQDTAIRVFAIPPS
FASIFLTKSTKLTCLVTDLTTYDSVTISWTRQNGEAVKTHTNISESHPNATFSAVGEAS
ICEDDWNSGERFTCTVTHTDLPSPLKQTISRPKGVALHRPDVYLLPPAREQLNLRESA
TITCLVTGFSPADVFVQWMQRGQPLSPEKYVTSAPMPEPQAPGRYFAHSILTVSEEE
WNTGETYTCVAHEALPNRVTERTVDKSTGKPTLYNVSLVMSDTAGTCY.
Human IgG4 constant region, Uniprot: P01861:
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPSCPAPEFLG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK.
Human IgA1 constant region, Uniprot: P01876:
ASPTSPKVFPLSLCSTQPDGNVVIACLVQGFFPQEPLSVTWSESGQGVTARNFPPSQD
ASGDLYTTSSQLTLPATQCLAGKSVTCHVKHYTNPSQDVTVPCPVPSTPPTPSPSTPP
TPSPSCCHPRLSLHRPALEDLLLGSEANLTCTLTGLRDASGVTFTWTPSSGKSAVQGP
PERDLCGCYSVSSVLPGCAEPWNHGKTFTCTAAYPESKTPLTATLSKSGNTFRPEVH
LLPPPSEELALNELVTLTCLARGFSPKDVLVRWLQGSQELPREKYLTWASRQEPSQG
TTTFAVTSILRVAAEDWKKGDTFSCMVGHEALPLAFTQKTIDRLAGKPTHVNVSVV
MAEVDGTCY.
Human IgA2 constant region, Uniprot: P01877:
ASPTSPKVFPLSLDSTPQDGNVVVACLVQGFFPQEPLSVTWSESGQNVTARNFPPSQD
ASGDLYTTSSQLTLPATQCPDGKSVTCHVKHYTNPSQDVTVPCPVPPPPPCCHPRLSL
HRPALEDLLLGSEANLTCTLTGLRDASGATFTWTPSSGKSAVQGPPERDLCGCYSVS
SVLPGCAQPWNHGETFTCTAAHPELKTPLTANITKSGNTFRPEVHLLPPPSEELALNE
LVTLTCLARGFSPKDVLVRWLQGSQELPREKYLTWASRQEPSQGTTTFAVTSILRVA
AEDWKKGDTFSCMVGHEALPLAFTQKTIDRMAGKPTHVNVSVVMAEVDGTCY.
Human Ig kappa constant region, Uniprot: P01834:
TVAAPSVFIFPPSDEQLKSGTASVVCLLNNFYPREAKVQWKVDNALQSGNSQESVTE
QDSKDSTYSLSSTLTLSKADYEKHKVYACEVTHQGLSSPVTKSFNRGEC

In some embodiments, the antibody or antigen binding fragment comprises and IgG1 constant domain.

In addition, the antibodies disclosed herein may be engineered to include modifications within the Fc region to alter one or more functional properties of the antibody, such as serum half-fife, complement fixation, Fc receptor binding, and/or antigen-dependent cellular cytotoxicity. Such modifications include, but are not limited to, alterations of the number of cysteine residues in the hinge region to facilitate assembly of the light and heavy chains or to increase or decrease the stability of the antibody (U.S. Pat. No. 5,677,425) and amino acid mutations in the Fc hinge region to decrease the biological half-life of the antibody (U.S. Pat. No. 6,165,745).

Additionally, the antibodies disclosed herein may be chemically modified. Glycosylation of an antibody can be altered, for example, by modifying one or more sites of glycosylation within the antibody sequence to increase the affinity of the antibody for antigen (U.S. Pat. Nos. 5,714,350 and 6,350,861). Alternatively, to increase antibody-dependent cell-mediated cytotoxicity, a hypofucosylated antibody having reduced amounts of fucosyl residues or an antibody having increased bisecting GlcNac structures can be obtained by expressing the antibody in a host cell with altered glycosylation mechanism (Shields, R. L. et al. (2002) J. Biol. Chem. 277:26733-26740; Umana et al. (1999) Nat. Biotech. 17:176-180).

The antibodies disclosed herein can be pegylated to increase biological half-life by reacting the antibody or fragment thereof with polyethylene glycol (PEG) or a reactive ester or aldehyde derivative of PEG, under conditions in which one or more PEG groups become attached to the antibody or antibody fragment. Antibody pegylation may be carried out by an acylation reaction or an alkylation reaction with a reactive PEG molecule (or an analogous reactive water soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. The antibody to be pegylated can be an aglycosylated antibody. Methods for pegylating proteins are known in the art and can be applied to the antibodies disclosed herein (EP 0154316 and EP 0401384).

Additionally, antibodies may be chemically modified by conjugating or fusing the antigen-binding region of the antibody to serum protein, such as human serum albumin, to increase half-life of the resulting molecule. Such approach is for example described in EP 0322094 and EP 0486525.

The antibodies or fragments thereof of the present disclosure may be conjugated to a diagnostic agent and used diagnostically, for example, to monitor the development or progression of a disease and determine the efficacy of a given treatment regimen. Examples of diagnostic agents include enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody or fragment thereof, or indirectly, through a linker using techniques known in the art. Examples of suitable enzymes include horseradish peroxidase, alkaline phosphatase, beta-galactosidase, or acetylcholinesterase. Examples of suitable prosthetic group complexes include streptavidin/biotin and avidin/biotin. Examples of suitable fluorescent materials include umbelliferone, fluorescein, fluorescein isothiocyanate, rhodamine, dichlorotriazinylamine fluorescein, dansyl chloride or phycoerythrin. An example of a luminescent material includes luminol. Examples of bioluminescent materials include luciferase, luciferin, and aequorin. Examples of suitable radioactive material include ¹²⁵I, ¹³¹I, Indium-111, Lutetium-171, Bismuth-212, Bismuth-213, Astatine-211, Copper-62, Copper-64, Copper-67, Yttrium-90, Iodine-125, Iodine-131, Phosphorus-32, Phosphorus-33, Scandium-47, Silver-111, Gallium-67, Praseodymium-142, Samarium-153, Terbium-161, Dysprosium-166, Holmium-166, Rhenium-186, Rhenium-188, Rhenium-189, Lead-212, Radium-223, Actinium-225, Iron-59, Selenium-75, Arsenic-77, Strontium-89, Molybdenum-99, Rhodium-1105, Palladium-109, Praseodymium-143, Promethium-149, Erbium-169, Iridium-194, Gold-198, Gold-199, and Lead-211. Monoclonal antibodies may be indirectly conjugated with radiometal ions through the use of bifunctional chelating agents that are covalently linked to the antibodies. Chelating agents may be attached through amities (Meares et al. (1984) Anal. Biochem. 142:68-78); sulfhydrl groups (Koyama (1994) Chem. Abstr. 120:217-262) of amino acid residues and carbohydrate groups (Rodwell et al. (1986) PNAS USA 83:2632-2636; Quadri et al. (1993) Nucl. Med. Biol. 20:559-570).

Further, the antibodies or fragments thereof of the present disclosure may be conjugated to a therapeutic agent. Suitable therapeutic agents include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin, antimetabolites (such as methotrexate, 6-mercaptopurine, 6-thioguanine, cytarabine, fludarabin, 5-fluorouracil, decarbazine, hydroxyurea, asparaginase, gemcitabinc, cladribine), alkylating agents (such as mechlorethamine, thioepa, chloramhucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin), antibiotics (such as dactinomycin (formerly actinomycin), bleomycin, daunorubicin (formerly daunomycin), doxorubicin, idarubicin, mithramycin, mitomycin, mitoxantrone, plicamycin, anthramycin (AMC)), diphtheria toxin and related molecules (such as diphtheria A chain and active fragments thereof and hybrid molecules), ricin toxin (such as ricin A or a deglycosylated ricin A chain toxin), cholera toxin, a Shiga-like toxin (SLT-I, SLT-II, SLT-IIV), LT toxin, C3 toxin, Shiga toxin, pertussis toxin, tetanus toxin, soybean Bowman-Birk protease inhibitor, Pseudomonas exotoxin, alorin, saporin, modeccin, gelanin, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrietocin, phenomycin, enomycin toxins and mixed toxins.

Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. Aptamers are small nucleic acids ranging from 15-50 bases in length that fold into defined secondary and tertiary structures, such as stem-loops or G-quartets, and can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Ribozymes are nucleic acid molecules that are capable of catalyzing a chemical reaction, either intramolecularly or intermolecularly. Ribozymes typically cleave nucleic acid substrates through recognition and binding of the target substrate with subsequent cleavage. Triplex forming function nucleic acid molecules can interact with double-stranded or single-stranded nucleic acid by forming a triplex, in which three strands of DNA form a complex dependent on both Watson-Crick and Hoogsteen base-pairing. Triplex molecules can bind target regions with high affinity and specificity.

The functional nucleic acid molecules may act as effectors, inhibitors, modulators, and stimulators of a specific activity possessed by a target molecule, or the functional nucleic acid molecules may possess a de novo activity independent of any other molecules.

The therapeutic agents can be linked to the antibody directly or indirectly, using any of a large number of available methods. For example, an agent can be attached at the hinge region of the reduced antibody component via disulfide bond formation, using cross-linkers such as N-succinyl 3-(2-pyridyldithio)proprionate (SPDP), or via a carbohydrate moiety in the Fc region of the antibody (Yu et al. 1994 Int. J. Cancer 56: 244; Upeslacis et al., “Modification of Antibodies by Chemical Methods,” in Monoclonal antibodies: principles and applications, Birch et al. (eds.), pages 187-230 (Wiley-Liss, Inc. 1995); Price, “Production and Characterization of Synthetic Peptide-Derived Antibodies,” in Monoclonal antibodies: Production, engineering and clinical application, Ritter et al. (eds.), pages 60-84 (Cambridge University Press 1995)).

Techniques for conjugating therapeutic agents to antibodies are well known (Amon et al. “Monoclonal Antibodies For Immunotargeting Of Drugs In Cancer Therapy,” in Monoclonal Antibodies And Cancer Therapy; Reisfeld et al. (eds.), pp. 243-56 (Alan R. Liss, Inc. 1985); Hellstrom et al. “Antibodies For Drug Delivery,” in Controlled Drug Delivery (2nd Ed.); Robinson et al. (eds.), pp. 623-53 (Marcel Dekker, Inc. 1987); Thorpe “Antibody Carriers Of Cytotoxic Agents In Cancer Therapy: A Review,” in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody in Cancer Therapy,” in Monoclonal Antibodies For Cancer Detection And Therapy, Baldwin et al. (eds.), pp. 303-16 (Academic Press 1985), and Thorpe et al. “The Preparation And Cytotoxic Properties Of Antibody-Toxin Conjugates,” (1982) Immunol. Rev. 62:119-58).

The antibodies disclosed herein or antigen-binding regions thereof can be linked to another functional molecule such as another antibody or ligand for a receptor to generate a bi-specific or multi-specific molecule that binds to at least two or more different binding sites or target molecules. Linking of the antibody to one or more other binding molecules, such as another antibody, antibody fragment, peptide or binding mimetic, can be done, for example, by chemical coupling, genetic fusion, or noncovalent association. Multi-specific molecules can further include a third binding specificity, in addition to the first and second target epitope.

Bi-specific and multi-specific molecules can be prepared using methods known in the art. For example, each binding unit of the hi-specific molecule can be generated separately and then conjugated to one another. When the binding molecules are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitroberizoic acid) (DTNB), o-phenylenedimaleimide (oPDM), N-succinimidyl-3-(2-pyridyldithio)propionate (SPDP), and sulfosuccinimidyl 4-(N-maleimidomethyl)cyclohaxane-I-carboxylate (sulfo-SMCC) (Karpovsky et al. (1984) J. Exp. Med. 160:1686; Liu et al. (1985) Proc. Natl. Acad. Sci. USA 82:8648). When the binding molecules are antibodies, they can be conjugated by sulfhydryl bonding of the C-terminus hinge regions of the two heavy chains.

The antibodies or fragments thereof of the present disclosure may be linked to a moiety that is toxic to a cell to which the antibody is bound to form “depleting” antibodies. These antibodies are particularly useful in applications where it is desired to deplete an NK cell.

The antibodies disclosed herein may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

The antibodies also can be bound to many different carriers. Thus, this disclosure also provides compositions containing the antibodies and another substance, active or inert. Examples of well-known carriers include glass, polystyrene, polypropylene, polyethylene, dextran, nylon, amylase, natural and modified cellulose, polyacrylamide, agarose, and magnetite. The nature of the carrier can be either soluble or insoluble for purposes disclosed herein. Those skilled in the art will know of other suitable carriers for binding monoclonal antibodies, or will be able to ascertain such, using routine experimentation.

Antibodies disclosed herein can be used to purify the polypeptides disclosed herein and to identify biological equivalent polypeptide and/or polynucleotides. They also can be used to identify agents that modify the function of the polypeptides disclosed herein. These antibodies include polyclonal antisera, monoclonal antibodies, and various reagents derived from these preparations that are familiar to those practiced in the art and described above.

Antibodies that neutralize the activities of proteins encoded by identified genes can also be used in vivo and in vitro to demonstrate function by adding such neutralizing antibodies into in vivo and in vitro test systems. They also are useful as pharmaceutical agents to modulate the activity of polypeptides disclosed herein.

Various antibody preparations can also be used in analytical methods such as ELISA assays or Western blots to demonstrate the expression of proteins encoded by the identified genes by test cells in vitro or in vivo. Fragments of such proteins generated by protease degradation during metabolism can also be identified by using appropriate polyclonal antisera with samples derived from experimental samples.

Compositions

Further provided are compositions comprising the recombinant polynucleotide, antibody, antibody fragment, vector or host cell or precursor of each as described above, and a detectable label and/or a carrier, e.g. a solid support or liquid carrier. In one aspect, the carrier is a pharmaceutically acceptable carrier.

In one aspect, the compositions are formulated with one or more pharmaceutically acceptable excipients, diluents, carriers and/or adjuvants. In addition, embodiments of the compositions of the present disclosure include one or more of an isolated polypeptide disclosed herein, an isolated polynucleotide disclosed herein, a vector disclosed herein, an isolated host cell disclosed herein, or an antibody of the disclosure, formulated with one or more pharmaceutically acceptable substances.

In a further aspect, the composition is formulated with an addition therapeutic agent selected for the disease to be treated, e.g., an anti-cancer, anti-inflammatory or an anti-autoimmune therapeutic. Non-limiting examples include chemotherapy, radiation, antibiotics, chalcones, curcumin, Virobay, Taxol, and/or inhibition of other proteins involved in tumor progression.

For oral preparations, any one or more of an isolated or recombinant polypeptide as described herein, an isolated or recombinant polynucleotide as described herein, a vector as described herein, an isolated host cell as described herein, a small molecule or an antibody as described herein can be used alone or in pharmaceutical formulations disclosed herein comprising, or consisting essentially of, the compound in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical formulations and unit dose forms suitable for oral administration are particularly useful in the treatment of chronic conditions, infections, and therapies in which the patient self-administers the drug. In one aspect, the formulation is specific for pediatric administration.

The disclosure provides pharmaceutical formulations in which the one or more of an isolated polypeptide disclosed herein, an isolated polynucleotide disclosed herein, a vector disclosed herein, an isolated host cell disclosed herein, or an antibody disclosed herein can be formulated into preparations for injection in accordance with the disclosure by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives or other antimicrobial agents. For intravenous administration, suitable carriers include physiological bacteriostatic water, Cremophor EL™ (BASF, Parsippany, N.J.), or phosphate buffered saline (PBS). In all cases, a composition for parenteral administration must be sterile and should be fluid to the extent that easy syringability exists.

Aerosol formulations provided by the disclosure can be administered via inhalation and can be propellant or non-propellant based. For example, embodiments of the pharmaceutical formulations disclosed herein comprise a compound disclosed herein formulated into pressurized acceptable propellants such as dichlorodifluoromethane, propane, nitrogen and the like. For administration by inhalation, the compounds can be delivered in the form of an aerosol spray from a pressurized container or dispenser which contains a suitable propellant, e.g., a gas such as carbon dioxide, or a nebulizer. A non-limiting example of a non-propellant is a pump spray that is ejected from a closed container by means of mechanical force (i.e., pushing down a piston with one's finger or by compression of the container, such as by a compressive force applied to the container wall or an elastic force exerted by the wall itself, e.g., by an elastic bladder).

Suppositories disclosed herein can be prepared by mixing a compound disclosed herein with any of a variety of bases such as emulsifying bases or water-soluble bases. Embodiments of this pharmaceutical formulation of a compound disclosed herein can be administered rectally via a suppository. The suppository can include vehicles such as cocoa butter, carbowaxes and polyethylene glycols, which melt at body temperature, yet are solidified at room temperature.

Unit dosage forms for oral or rectal administration, such as syrups, elixirs, and suspensions, may be provided wherein each dosage unit, for example, teaspoonful, tablespoonful, tablet or suppository, contains a predetermined amount of the composition containing one or more compounds disclosed herein. Similarly, unit dosage forms for injection or intravenous administration may comprise a compound disclosed herein in a composition as a solution in sterile water, normal saline or another pharmaceutically acceptable carrier.

Embodiments of the pharmaceutical formulations disclosed herein include those in which one or more of an isolated polypeptide disclosed herein, an isolated polynucleotide disclosed herein, a vector disclosed herein, a small molecule for use in the disclosure, an isolated host cell disclosed herein, or an antibody disclosed herein is formulated in an injectable composition. Injectable pharmaceutical formulations disclosed herein are prepared as liquid solutions or suspensions; or as solid forms suitable for solution in, or suspension in, liquid vehicles prior to injection. The preparation may also be emulsified or the active ingredient encapsulated in liposome vehicles in accordance with other embodiments of the pharmaceutical formulations disclosed herein.

In an embodiment, one or more of an isolated polypeptide disclosed herein, an isolated polynucleotide disclosed herein, a vector disclosed herein, an isolated host cell disclosed herein, or an antibody disclosed herein is formulated for delivery by a continuous delivery system. The term “continuous delivery system” is used interchangeably herein with “controlled delivery system” and encompasses continuous (e.g., controlled) delivery devices (e.g., pumps) in combination with catheters, injection devices, and the like, a wide variety of which are known in the art.

Mechanical or electromechanical infusion pumps can also be suitable for use with the present disclosure. Examples of such devices include those described in, for example, U.S. Pat. Nos. 4,692,147; 4,360,019; 4,487,603; 4,360,019; 4,725,852; 5,820,589; 5,643,207; 6,198,966; and the like. In general, delivery of a compound disclosed herein can be accomplished using any of a variety of refillable, pump systems. Pumps provide consistent, controlled release over time. In some embodiments, a compound disclosed herein is in a liquid formulation in a drug-impermeable reservoir, and is delivered in a continuous fashion to the individual.

In one embodiment, the drug delivery system is an at least partially implantable device. The implantable device can be implanted at any suitable implantation site using methods and devices well known in the art. An implantation site is a site within the body of a subject at which a drug delivery device is introduced and positioned. Implantation sites include, but are not necessarily limited to, a subdermal, subcutaneous, intramuscular, or other suitable site within a subject's body. Subcutaneous implantation sites are used in some embodiments because of convenience in implantation and removal of the drug delivery device.

Drug release devices suitable for use in the disclosure may be based on any of a variety of modes of operation. For example, the drug release device can be based upon a diffusive system, a convective system, or an erodible system (e.g., an erosion-based system). For example, the drug release device can be an electrochemical pump, osmotic pump, an electroosmotic pump, a vapor pressure pump, or osmotic bursting matrix, e.g., where the drug is incorporated into a polymer and the polymer provides for release of drug formulation concomitant with degradation of a drug-impregnated polymeric material (e.g., a biodegradable, drug-impregnated polymeric material). In other embodiments, the drug release device is based upon an electrodiffusion system, an electrolytic pump, an effervescent pump, a piezoelectric pump, a hydrolytic system, etc.

Drug release devices based upon a mechanical or electromechanical infusion pump can also be suitable for use with the present disclosure. Examples of such devices include those described in, for example, U.S. Pat. Nos. 4,692,147; 4,360,019; 4,487,603; 4,360,019; 4,725,852; and the like. In general, a subject treatment method can be accomplished using any of a variety of refillable, non-exchangeable pump systems. Pumps and other convective systems may be utilized due to their generally more consistent, controlled release over time. Osmotic pumps are used in some embodiments due to their combined advantages of more consistent controlled release and relatively small size (see, e.g., PCT International Application Publication No. WO 97/27840 and U.S. Pat. Nos. 5,985,305 and 5,728,396). Exemplary osmotically-driven devices suitable for use in the disclosure include, but are not necessarily limited to, those described in U.S. Pat. Nos. 3,760,984; 3,845,770; 3,916,899; 3,923,426; 3,987,790; 3,995,631; 3,916,899; 4,016,880; 4,036,228; 4,111,202; 4,111,203; 4,203,440; 4,203,442; 4,210,139; 4,327,725; 4,627,850; 4,865,845; 5,057,318; 5,059,423; 5,112,614; 5,137,727; 5,234,692; 5,234,693; 5,728,396; and the like. A further exemplary device that can be adapted for the present disclosure is the Synchromed infusion pump (Medtronic).

In some embodiments, the drug delivery device is an implantable device. The drug delivery device can be implanted at any suitable implantation site using methods and devices well known in the art. As noted herein, an implantation site is a site within the body of a subject at which a drug delivery device is introduced and positioned. Implantation sites include, but are not necessarily limited to a subdermal, subcutaneous, intramuscular, or other suitable site within a subject's body.

Suitable excipient vehicles for a compound disclosed herein are, for example, water, saline, dextrose, glycerol, ethanol, or the like, and combinations thereof. In addition, if desired, the vehicle may contain minor amounts of auxiliary substances such as wetting or emulsifying agents or pH buffering agents. Methods of preparing such dosage forms are known, or will be apparent upon consideration of this disclosure, to those skilled in the art. See, e.g., Remington's Pharmaceutical Sciences, Mack Publishing Company, Easton, Pa., 17th edition, 1985. The composition or formulation to be administered will, in any event, contain a quantity of the compound adequate to achieve the desired state in the subject being treated.

Compositions of the present disclosure include those that comprise a sustained-release or controlled release matrix. In addition, embodiments of the present disclosure can be used in conjunction with other treatments that use sustained-release formulations. As used herein, a sustained-release matrix is a matrix made of materials, usually polymers, which are degradable by enzymatic or acid-based hydrolysis or by dissolution. Once inserted into the body, the matrix is acted upon by enzymes and body fluids. A sustained-release matrix desirably is chosen from biocompatible materials such as liposomes, polylactides (polylactic acid), polyglycolide (polymer of glycolic acid), polylactide co-glycolide (copolymers of lactic acid and glycolic acid), polyanhydrides, poly(ortho)esters, polypeptides, hyaluronic acid, collagen, chondroitin sulfate, carboxcylic acids, fatty acids, phospholipids, polysaccharides, nucleic acids, polyamino acids, amino acids such as phenylatanine, tyrosine, isoleucine, polynucleotides, polyvinyl propylene, polyvinylpyrrolidone and silicone. Illustrative biodegradable matrices include a polylactide matrix, a polyglycolide matrix, and a polylactide co-glycolide (co-polymers of lactic acid and glycolic acid) matrix.

In another embodiment, the interfering agent (as well as combination compositions) is delivered in a controlled release system. For example, a compound disclosed herein may be administered using intravenous infusion, an implantable osmotic pump, a transdermal patch, liposomes, or other modes of administration. In one embodiment, a pump may be used (Sefton (1987) CRC Crit. Ref. Biomed. Eng. 14:201; Buchwald et al. (1980) Surgery 88:507; Saudek et al. (1989) N. Engl. J. Med. 321:574). In another embodiment, polymeric materials are used. In yet another embodiment a controlled release system is placed in proximity of the therapeutic target, i.e., the liver, thus requiring only a fraction of the systemic dose. In yet another embodiment, a controlled release system is placed in proximity of the therapeutic target, thus requiring only a fraction of the systemic. Other controlled release systems are discussed in the review by Langer (1990) Science 249:1527-1533.

In another embodiment, the compositions of the present disclosure (as well as combination compositions separately or together) include those formed by impregnation of a compound described herein into absorptive materials, such as sutures, bandages, and gauze, or coated onto the surface of solid phase materials, such as surgical staples, zippers and catheters to deliver the compositions. Other delivery systems of this type will be readily apparent to those skilled in the art in view of the instant disclosure.

The compositions can be used for screening and purification of naturally occurring products by reliance on their inherent properties to recognize and bind binding partners. They also can be used in vitro and in vivo to inhibit human cathepsin B activity or treating a condition or disease linked to cathepsin B expression in a subject, by contacting the cathepsin B or a sample suspected of containing the cathepsin B with an effective amount of the recombinant antibody. In one aspect the disease is cancer, e.g., metastatic cancer and the therapy can be administered as a first line, second line, third line, fourth line or fifth line therapy. In can be combined with a companion diagnostic to determine if the patient overexpresses cathepsin and then administering the therapy to that patient. Any appropriate means for determining the expression level of cathepsin or other appropriate marker is within the scope of this disclosure.

Diagnostic, Therapeutic, and Screening Methods

Also provided is a method for inhibiting cathepsin B activity in a subject in need thereof, comprising, or alternatively consisting essentially of, or yet further consisting of administering to the subject an effective amount of the recombinant antibody or antigen binding fragments disclosed herein. One of skill in the art can determine when cathepsin B activity has been inhibited by taking a sample suspected of containing cathepsin B and contacting the sample with an effective amount of a peptide substrate and determining if the substrate was acted upon by any cathepsin B in the sample. An example of such an assay is disclosed herein and others are commercially available.

Further provided is a method for treating a condition mediated by cathepsin B activity in a subject in need thereof, comprising, or alternatively consisting essentially of, or consisting of, administering an effective amount of the recombinant antibody as described herein.

In a further aspect, the therapy is combined with an additional therapeutic agent selected for the disease to be treated, e.g., an anti-cancer, anti-inflammatory or an anti-autoimmune therapeutic. Non-limiting examples include chemotherapy, radiation, antibiotics, chalcones, curcumin, Virobay, Taxol, and/or inhibition of other proteins involved in tumor progression. The administration is concurrent or sequential and the effective amount and dosing schedule is determined by the treating physical or professsional.

Conditions treated by the method are abnormal conditions related to the expression of the protein, non-limiting examples of such include inflammation, infection, cancer, metastates, metastatic potential of cancer cell, melanoma, breast cancer, oral cancer, ovarian cancer, pancreatic cancer, lung cancer, liver cancer, rheumatoid arthritis, and osteoarthrisis.

In one aspect, the subject is a mammal, e.g., a human patient and the condition is cancer. In another aspect, the subject is a non-human subject and the administration is for veterinary purposes.

The recombinant antibody or antigen binding fragments disclosed herein as disclosed herein may be administered either alone or in combination with diluents, known anti-cancer therapeutics, and/or with other components such as cytokines or other cell populations that are immunostimulatory. They may be administered as a first line therapy, a second line therapy, a third line therapy, or further therapy. Non-limiting examples of additional therapies include chemotherapeutics or biologics. Appropriate treatment regimens will be determined by the treating physician or veterinarian.

Methods for determining if a treatment has been successful are known in the art and comprise for example, clinical and subclinical endpoints, a reduction in tumor mass or metastasis, a reduction in infection or inflammation.

Suitable clinical endpoints and surrogate endopoints for cancer are disclosed herein, e.g. inhibition or arrest of metastasis, reduction in the size of tumor mass or masses (for example greater than or about a 20%, 25%, 30%, 35%, 40%, >45%, 50%, 55%, 60%, 65%, 70%, 75%, 80%, 85%, 90%, 95%, 96%, 97%, 98%, or 99% reduction in the size of tumor size or masses), long term absence of symptoms or signs of disease (in some cases, even in the presence of viable tumor cells), remission or disappearance of clinical symptoms, stable disease (neither improvement or worsening), longer interval between resolution of symptoms and relapse or death, and either disease free or progression feel survival.

Pharmaceutical compositions of the present invention may be administered in a manner appropriate to the disease to be treated or prevented. The quantity and frequency of administration will be determined by such factors as the condition of the patient, and the type and severity of the patient's disease, although appropriate dosages may be determined by clinical trials.

The methods can be combined with a screen to determine if the patient is suitable for therapy by determining the cathepsin B level in the patient and then if the protein is overexpressed, administering the therapy. Methods to determine cathepsin B levels are known in the art.

The present disclosure also provides methods for screening for equivalent agents, such as equivalent antibodies and antigen binding fragments described herein and various agents that modulate the activity of the function of a polypeptide or peptide, e.g. cathepsin B. For the purposes of this disclosure, an “agent” is intended to include, but not be limited to a biological or chemical compound such as a simple or complex organic or inorganic molecule, a peptide, a protein (e.g., antibody), a polynucleotide anti-sense) or a ribozyme. A vast array of compounds can be synthesized, for example polymers, such as polypeptides and polynucleotides, and synthetic organic compounds based on various core structures, and these are also included in the term “agent.” In addition, various natural sources can provide compounds for screening, such as plant or animal extracts, and the like. It should be understood, although not always explicitly stated that the agent is used alone or in combination with another agent, having the same or different biological activity as the agents identified by the inventive screen.

One embodiment is a method for screening agents capable of interacting with and/or inhibiting cathepsin B proteolytic activity. The present disclosure provides the three-dimensional structure of stefin A, the cathepsin B, the propeptide in cathepsin B zymogen, and one or more antibodies disclosed herein. Accordingly, the disclosure permits the use of virtual design techniques, also known as computer-aided, in silico design or modeling, to design, select, and synthesize agents capable of interacting with and/or inhibiting cathepsin B. In turn, the candidate agents may be effective in the method aspects disclosed herein above. Thus, the present disclosure also provides agents identified or designed by the in silico methods.

In addition to the computer-implemented methods as provided herein, the present disclosure also provides custom computer system that includes, e.g., processor, memory and/or program, for performing the methods, as well as a computer readable medium, such as a non-transitory computer readable medium that stores suitable computer program or code for carrying out the methods.

Accordingly, another embodiment provides a custom computing apparatus comprising:
at least one processor;
a memory coupled to the at least one processor;
a storage medium in communication with the memory and the at least one processor, the storage medium containing a set of processor executable instructions that, when executed by the processor configure the custom computing apparatus to identify an agent capable of interacting with and/or inhibiting cathepsin , wherein the configuration comprises:
positioning a three-dimensional structure of a candidate agent against a three-dimensional structure of stefin A, cathepsin B, propeptide of cathepsin B zymogen, and one or more antibodies disclosed herein and identifying that the agent inhibits cathepsin B proteolytic activity based on structural similarities or correspondence to said three-dimensional structure.

Yet another embodiment provides a non-transitory computer medium comprising a set of processor executable instructions that, when executed by a processor, identifying an agent according to the above disclosed method.

Methods of in silico molecule or drug designs are well known in the art, see generally Kapetanovic (2008) Chem Biol. Interact., 171(2):165-76. Briefly, the atomic coordinates of the three-dimensional structure are input into a computer so that images of the structure and various parameters are shown on the display. The design typically involves positioning a three-dimensional structure to the three-dimensional structure of the target molecule. The positioning can be controlled by the user with assistance from a computer's graphic interface, and can be further guided by a computer algorithm looking for potential good matches. Positioning also involves moving either or both of the three-dimensional structures around at any dimension.

Then, the resultant data are input into a virtual compound and/or agent library. Since a virtual library is contained in a virtual screening software such as DOCK-4 (Kuntz, UCSF), the above-described data may be input into such a software. Candidate agents may be searched for, using a three-dimensional structure database of virtual or non-virtual drug candidate compounds, such as MDDR (Prous Science, Spain).

A candidate agent is found to be able to bind to cathepsin B if a desired interaction between the candidate agent and cathepsin B is found. The interaction can be quantitative, e.g., strength of interaction and/or number of interaction sites, or qualitative, e.g., interaction or lack of interaction. The output of the method, accordingly, can be quantitative or qualitative. In one aspect, therefore, the present disclosure also provides a method for identifying an agent that does not inhibit the interaction or alternatively, strengthens the interaction between the DNA and protein.

The potential inhibitory or binding effect (i.e., interaction or association) of an agent such as a small molecule compound may be analyzed prior to its actual synthesis and testing by the use of computer modeling techniques. If the theoretical structure of the given compound suggests insufficient interaction and association between it and cathepsin B, synthesis and testing of the agent can be obviated. However, if computer modeling indicates a strong interaction, the agent can then be synthesized and tested for its ability to bind to or inhibit the interaction using various methods such as in vitro or in vivo experiments. Methods of testing an agent's ability to inhibit or titrate a biofilm, alone or in connection with another agent, are disclosed herein. In this manner, synthesis of inoperative agents and compounds can be avoided.

One skilled in the art may use any of several methods to screen chemical or biological entities or fragments for their ability to associate with cathepsin B and more particularly with the specific binding sites. Selected fragments or chemical entities may then be positioned in a variety of orientations, or docked, within an individual binding site of cathepsin B. Docking may be accomplished using software such as QUANTA, SYBYL, followed by energy minimization and molecular dynamics with standard molecular mechanics force fields, such as CHARMM and AMBER.

Commercial computer programs are also available for in silico design. Examples include, without limitation, GRID (Oxford University, Oxford, UK), MCSS (Molecular Simulations, Burlington, Mass.), AUTODOCK (Scripps Research Institute, La Jolla, Calif.), DOCK (University of California, San Francisco, Calif.), GLIDE (Schrodinger Inc.), FlexX (Tripos Inc.) and GOLD (Cambridge Crystallographic Data Centre).

Once an agent or compound has been designed or selected by the above methods, the efficiency with which that agent or compound may bind to each other can be tested and optimized by computational evaluation. For example, an effective agent or may demonstrate a relatively small difference in energy between its bound and free states (i.e., a small deformation energy of binding).

A compound designed or selected can be further computationally optimized so that in its bound state it may optionally lack repulsive electrostatic interaction with the target protein. Such non-complementary (e.g., electrostatic) interactions include repulsive charge-charge, dipole-dipole, and charge-dipole interactions. Specifically, the sum of all electrostatic interactions between the agent and cathepsin B when the agent or compound is bound to either agent, optionally making a neutral or favorable contribution to the enthalpy of binding.

Computer software are also available in the art to evaluate compound deformation energy and electrostatic interaction. Examples include, without limitation, Gaussian 92 [Gaussian, Inc., Pittsburgh, Pa.]; AMBER [University of California at San Francisco]; QUANTA/CHARMM [Molecular Simulations, Inc., Burlington, Mass.]; and Insight II/Discover [Biosysm Technologies Inc., San Diego, Calif.].

Once an binding agent has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit to cathepsin B by the same computer methods described in detail, above.

Certain embodiments relate to a method for screening small molecules capable of interacting with the protein or polynucleotide disclosed herein. For the purpose of this disclosure, “small molecules” are molecules having low molecular weights (MW) that are, in one embodiment, capable of binding to a protein of interest thereby altering the function of the protein. In some embodiments, the MW of a small molecule is no more than 1,000. Methods for screening small molecules capable of altering protein function are known in the art. For example, a miniaturized arrayed assay for detecting small molecule-protein interactions in cells is discussed by You et al. (1997) Chem. Biol. 4:961-968.

To practice the screening method in vitro, suitable cell culture or tissue infected with the microbial to be treated are first provided. The cells are cultured under conditions (temperature, growth or culture medium and gas (CO₂)) and for an appropriate amount of time to attain exponential proliferation without density dependent constraints. It also is desirable to maintain an additional separate cell culture that is not infected as a control.

As is apparent to one of skill in the art, suitable cells can be cultured in micro-titer plates and several agents can be assayed at the same time by noting genotypic changes, phenotypic changes or a reduction in microbial titer.

When the agent is a composition other than a DNA or RNA, such as a small molecule as described above, the agent can be directly added to the cell culture or added to culture medium for addition. As is apparent to those skilled in the art, an “effective” a mount must be added which can be empirically determined,

When the agent is an antibody or antigen binding fragment, the agent can be contacted or incubated with the target antigen and polyclonal antibody as described herein under conditions to perform a competitive ELISA. Such methods are known to the skilled artisan.

The assays also can be performed in a subject. When the subject is an animal such as a rat, chinchilla, mouse or simian, the method provides a convenient animal model system that can be used prior to clinical testing of an agent in a human patient. In this system, a candidate agent is a potential drug if symptoms of the disease or microbial infection is reduced or eliminated, each as compared to untreated, animal having the same infection. It also can be useful to have a separate negative control group of cells or animals that are healthy and not treated, which provides a basis for comparison.

The agents and compositions can be used in the manufacture of medicaments and for the treatment of humans and other animals by administration in accordance with conventional procedures, such as an active ingredient in pharmaceutical compositions.

### EXAMPLES

The following examples are intended to illustrate, and not limit the embodiments disclosed herein.

### Example 1

### Stefin A-Derived Antibody and Propeptide Antibody Fusion Proteins that Potently Inhibit Cathepsin

Stefin A, a protein encoded by human genome, potently inhibits the proteolytic activity of cathepsin B by forming a tight complex. X-ray crystal structure revealed that stefin A functions through directly inserting a central loop into the active site of cathepsin B to inhibit its activity. In addition, two adjacent loops form extensive interactions with residues in close proximity to the protease active site. The x-ray crystal structure of the humanized anti-HER2 receptor monoclonal antibody trastuzumab, (its traded name Herceptin) shows that CDR loops exhibit similar conformation to those of inhibitory peptide modules, suggesting that CDR regions of Herceptin can be replaced with those inhibition loops to generate potent human monoclonal antibodies specifically inhibiting cathepsin B. As disclosed herein, those three interacting loops were genetically grafted (by recombinant techniques) into light chain of Herceptin Fab domain to substitute for its CDR loops. The major inhibition loop was substituted for the light chain CDR3, while the other two interacting loops were placed into adjacent light chain CDR2 and CDR1 loops, respectively. To promote correct folding of antibody chimeras and optimize conformation of the fused peptides for mimicking that of native inhibition modules in stefin A, flexible GGS linkers were inserted between the grafted peptides and the immunoglobulin scaffold. The resulting human monoclonal antibody was successfully expressed in Escherichia coli cells. Secreted antibody protein was characterized by SDS-PAGE gel stained with coomassie blue, showing expected molecular weight. Using recombinant human cathepsin B and a fluorogenic peptide substrate, inhibition activity of the generated antibody was characterized. The designed antibody potently inhibits proteolytic activity of human cathepsin B in a dose-dependent fashion. The structures of stefin A and Herceptin are available and attached. The structural model and sequences (DNA and amino acids) of the invented stefin A-derived antibody are also attached.

Generation of Stefin A-based antibody inhibitors against Cathepsin B. Through overlap extension PCR, three inhibitory loop regions of stefin A were genetically substituted for Herceptin Fab complementary determining regions (CDRs) light chain. The stefin A loop 1 was first reversed (H₂N-LGGPI-COOH; DNA sequence: CTGGGAGGTCCGATT) and grafted between His91 and Thr93 in Herceptin light chain CDR3. The stefin A loop 2 (H₂N-VVAGT-COOH; DNA sequence: GTCGTAGCGGGTACT) was inserted between Asn30 and THR31 in Herceptin light chain CDR1. The Stefin A loop 3 with a GGS linker at the N- and C-termini (H₂N-GGSKSLPGQNEDLSGG-COOH; DNA sequence:

GGGGGCTCTAAAAGCCTCCCTGGGCAGAACGAAGATCTGAGCGGGGGT)

was inserted between Tyr49 and Phe53 in Herceptin light chain CDR2. The amplified full-length insert and pBAD vector backbone were digested by NheI-HF and SalI-HF restriction enzymes (New England Biolabs, MA) at 37° C. for 3 hours, followed by DNA gel extraction. Using T4 DNA ligase (New England Biolabs, MA), the chimeric gene was in-frame ligated into pBAD backbone vector, resulting in bacterial expression vector of stefin A-derived antibody. The generated pBAD expression vector was confirmed by DNA sequencing.

### Example 2

### Expression and Purification of Stefin A-Derived Antibody

The protein was expressed in DH10B Escherichia coli (E. coli) transformed with pBAD expression vector by electroporation. Overnight bacterial culture in LB-broth was diluted to 1 liter culture medium and grown at 37° C. until OD_{600 nm} of 0.6. The antibody expression was induced with 0.02% L-arabinose and grown at 25° C. overnight with a shaking speed of 180 rpm. Expressed antibody was secreted into periplasm. Cells were harvested 24 hours post induction. Bacterial periplasma was lysed with lysis buffer (20% sucrose, 30 mM Tris-HCl pH 8, 1 mM EDTA, and 0.2 mg ml⁻¹ lysozyme) for 1 hour while shaking at 180 rpm. After centrifugation at 15,000 rpm at 4° C. for 30 minutes, the supernatant was then loaded onto protein G chromatography (Thermo Fisher Scientific, IL) for purification of the expressed antibody. The purity of antibody was examined by SDS-PAGE gel.

### Example 3

### Generation of Protease Propeptide-Fused Antibody

The gene encoding the propeptide of cathepsin B (CatB) was synthesized by IDT (Coralville, Iowa). The amino acid and DNA sequences for the propeptide are H₂N-RSRPSFHPLSDELVNYVNKRNTTWQAGHNFYNVDMSYLKRLCGTFLGGPKPPQRVMFTEDL-COOH and CGTTCTCGTCCGTCTTTCCACCCGCTGTCTGACGAACTGGTTAACTACGTTAACAA ACGTAACACCACCTGGCAGGCTGGTCACAACTTCTACAACGTTGACATGTCTTAC CTGAAACGTCTGTGCGGTACCTTCCTGGGTGGTCCGAAACCGCCGCAGCGTGTTA TGTTCACCGAAGACCTG, respectively. Herceptin light chain with the N-terminal propeptide fusion was generated by overlap extension PCR. Using T4 DNA ligase (New England Biolabs, MA), the chimeric gene was in-frame ligated into pFuse mammalian expression vector. Both the insert and the pFuse backbone vector were digested by EcoRI-HF and NheI-HF restriction enzymes (New England Biolabs, MA) at 37° C. according to the instructions by manufacturer, followed by DNA gel extraction. The ligation product was then used for electroporation of DH10B. The vector expressing Herceptin heavy chain with C-terminal propeptide fusion was obtained through the same strategy as described above. The obtained vectors were confirmed by DNA sequencing.

### Example 4

### Expression and Purification of Protease Propeptide-Fused Antibodies

Antibody fusion proteins were expressed in free-style HEK 293 cells transiently transfected with pFUSE expression vectors. The expressed proteins were secreted into culture media. HEK 293 suspension cells were cultured in freestyle 293 expression medium (Life Techonologies, CA) in shaker flasks at 37° C., 5% CO₂ and 125 rpm. To generate Herceptin light chain-CatB Propeptide fusion protein, 20 μg of pFUSE vector of Herceptin heavy chain and 10 μg vector of Herceptin light chain-CatB propeptide fusion protein gene were combined and then mixed with 60 μl 293fectin (Life Technologies, CA) for transfection of 30 millions of freestyle HEK 293 cells. To produce Herceptin heavy chain-CatB propeptide C-terminal fusion protein, 10 μg of vector expressing Herceptin light chain and 20 μg of vector expressing Herceptin heavy chain-CatB propeptide C-terminal Fusion Protein were combined for transfection. The culture media were collected at 48 hours and 96 hours after transfection then and subjected to Protein G chromatography (Thermo Fisher Scientific, IL).

### Example 5

### In Vitro Cathepsin B Enzymatic Activity Inhibition Assay

The inhibition assays were performed in 96-well plates at room temperature. Each well contained 30 μM of 7-amino-4-methylcoumarin (Z-Phe-Arg-AMC (R&D, MN)), 0.4 nM of Recombinant Human Procathepsin B (Novoprotein, NJ), and varied concentrations of fusion proteins. The final volume was 200 μl. The Z-Phe-Arg-AMC and Procathepsin B were dissolved in 160 μl activation buffer (340 mM sodium acetate, 60 mM acetic acid, 4 mM EDTA, pH 5) prior to addition of 40 μl of antibody inhibitors in PBS, pH 7.4. Through 5-minute incubation in the activation buffer, procathepsin B was converted into active form. The proteolytic activity of Cathepsin B was measured by monitoring the increase of fluorescence intensity at 460 nm (excitation at 380 nm), which was produced from the 7-amino-4methyl coumarin (AMC) upon cleavage of the amide bond of Z-Phe-Arg-AMC by cathepsin B.

### Example 6

### Generation of Protease Propeptide-Fused Antibody

The gene encoding the propeptide of cathepsin B (CatB) was synthesized by IDT (Coralville, Iowa). The amino acid and DNA sequences for the propeptide are H₂N-RSRPSFHPLSDELVNYVNKRNTTWQAGHNFYNVDMSYLKRLCGTFLGGPKPPQRVMFTEDL-COOH and CGTTCTCGTCCGTCTTTCCACCCGCTGTCTGACGAACTGGTTAACTACGTTAACAA ACGTAACACCACCTGGCAGGCTGGTCACAACTTCTACAACGTTGACATGTCTTAC CTGAAACGTCTGTGCGGTACCTTCCTGGGTGGTCCGAAACCGCCGCAGCGTGTTA TGTTCACCGAAGACCTG, respectively. Herceptin heavy chain with the N-terminal propeptide fusion (Provided in the sequence listing below) can be generated by overlap extension PCR. Similarly, Herceptin heavy chain with the C-terminal propeptide fusion (Provided in the sequence listing below) can be created by overlap extension PCR. To fuse the propeptide into CDR3 of Herceptin heavy chain (Provided in the sequence listing below), a coiled-coil structural motif is exploited for mediating the fusion of the propeptide to heavy chain CDR3 loop. The DNA sequences of the coiled-coil “stalk” (ascending strand: H₂N-GGSGAKLAALKAKLAALK-COOH and descending strand:

H₂N-
ELAALEAELAALEAGGSG-COOH) are
GGCGGAAGCGGAGCAAAGCTCGCCGCACTGAAAGCCAAGCTGGCCGCTCT
GAAG
and
GAACTGGCCGCACTGGAAGCTGAGCTGGCTGCCCTCGAAGCTGGAGGCTC
TGGA.

The Factor Xa recognition site (Ile-Glu-Gly-Arg) at the N-terminus of the propeptide fragment and the two Cys residues at the top of the coiled-coil “stalk” (H₂N-GGSGAKLAALKAKLAALKC-COOH and H₂N-CELAALEAELAALEAGGSG-COOH) are inserted by overlap extension PCR. Using T4 DNA ligase (New England Biolabs, MA), the chimeric genes are in-frame ligated into pFuse mammalian expression vector. Both the inserts and the pFuse backbone vector are digested by EcoRI-HF and NheI-HF restriction enzymes (New England Biolabs, MA) at 37° C. according to the instructions by manufacturer, followed by DNA gel extraction. The ligation products are then used for electroporation of DH10B. The obtained vectors are confirmed by DNA sequencing.

### Example 7

### Generation of Stefin A-Based Antibody Inhibitors Against Cathepsin B

Through overlap extension PCR, three inhibitory loop regions of stefin A are genetically substituted for Herceptin Fab complementary determining regions (CDRs) heavy chain. The stefin A loop 1 was first reversed (H₂N-LGGPI-COOH; DNA sequence: CTGGGAGGTCCGATT) and grafted between Gly101 and Gly103 in Herceptin heavy chain CDR3. The stefin A loop 2 (H₂N-VVAGT-COOH; DNA sequence: GTCGTAGCGGGTACT) can be inserted between Asp31 and Thr32 in Herceptin heavy chain CDR1. The Stefin A loop 3 with a GGS linker at the N- and C-termini (H₂N-GGSKSLPGQNEDLSGG-COOH; DNA sequence:

GGGGGCTCTAAAAGCCTCCCTGGGCAGAACGAAGATCTGAGCGGGGGT)

can be inserted between Ile51 and Gly56 in Herceptin heavy chain CDR2.

In addition, a few more antibodies can be designed by fusing the inhibitory loops of Stefin A to the different loops in Herceptin hypervariable regions. Through overlap extension PCR, two inhibitory loop regions of stefin A are genetically substituted for Herceptin Fab loops in light chain (Stefin A Loop X, Seq ID #1 in attached sequence listing). Stefin A loop 3 with GGS linker at both ends:

NH₂-GGSKSLPGQNEDLSGG-COOH (DNA: 5′-
GGGGGCTCTAAAAGCCTCCCTGGGCAGAACGAAGATCTGAGCGGGGGT-3′)

can be inserted into the CDR 2 of Herceptin Light chain in between Tyr 49 and Phe 53.

The Stefin A loop 2 amino acid sequence is reversed into NH₂-KIYYNTGAVVQTKYQ-COOH (DNA: 5′-AAGATATATTACAATACTGGAGCGGTTGTGCAAACCAAGTATCAA-3′) and can be inserted into a loop of Herceptin light chain in between Arg 66 and Phe 71.

Alternatively, through overlap extension PCR, two inhibitory loop regions of stefin A can be genetically substituted for Herceptin Fab CDR loops of light chain with coiled-coil structural motifs (Stefin A Coiled-coil, Seq ID #2 in sequence listing). Stefin A loop 2 together with coil-coil linkers at its N and C terminal NH₂-GGSGAKLAALKAKLAALKGGGGSKTQVVAGTNYYGGGGSELAALEAELAALEAGGSG-COOH (DNA: 5′-GGGGGTTCCGGCGCGAAGTTAGCGGCATTAAAAGCTAAACTCGCGGCTCTCAAA GGTGGAGGTGGCAGCAAAACCCAGGTCGTAGCGGGTACTAACTACTACGGTGGC GGCGGATCGGAACTTGCTGCGTTGGAAGCGGAACTTGCGGCGCTGGAAGCCGGT GGGAGTGGC-3′) are inserted into Herceptin light chain CDR1 in between Gln 27 and Asn 30. Stefin A loop 3 sequence with coil-coil at its N and C terminal:

NH₂-
GGSGAKLAALKAKLAALKGGGGSKVFKSLPGQNEDLVLTGGGGSELAALE
AELAALEAGGSG-COOH (DNA: 5′-
GGCGGCTCTGGAGCAAAATTGGCTGCATTAAAGGCGAAACTGGCAGCACT
GAAAGGTGGCGGTGGTAGTAAAGTTTTCAAAAGCCTCCCTGGGCAGAACG
AAGATCTGGTTCTGACCGGCGGAGGCGGATCGGAGCTGGCAGCCTTGGAA
GCCGAACTCGCCGCACTTGAAGCGGGAGGTAGCGGC-3′)

are inserted into Herceptin light chain CDR 3 in between His 91 and Thr 94.

In another design, through overlap extension PCR, three inhibitory loop regions of stefin A are genetically substituted for Herceptin Fab CDR loops of light chain (Stefin A CDR2 and 3 Swap, in sequence listing, below). Stefin A loop 2: NH₂-VVAGT-COOH (DNA: 5′-GTCGTAGCGGGTACT-3′) are inserted into Herceptin light chain CDR 1 in between Asn 30 and Thr 31. Stefin A loop 1 amino acid sequence is reversed into NH₂-LGGPI-COOH (DNA: 5′-CTGGGAGGTCCGATT-3′) and can be inserted into Herceptin light chain CDR 2 in between Ser 50 and Ala 51. Stefin A loop 3: NH₂-KSLPGQNEDL-COOH (DNA: 5′-AAAAGCCTCCCTGGGCAGAACGAAGATCTG-3′) is inserted into Herceptin light chain CDR 3 in between Gln 90 and Thr 97.

An antibody with extended stefin A loops was also generated. Through overlap extension PCR, three inhibitory loop regions of stefin A are genetically substituted for Herceptin Fab CDR loops of light chain (Stefin A CDR2 Ext, Seq ID #4 in sequence listing). Stefin A loop 2: NH₂-VVAGT-COOH (DNA: 5′-GTCGTAGCGGGTACT-3′) can be inserted into Herceptin light chain CDR 1 in between Asn 30 and Thr 31. Stefin A loop 3 with GGS linker at its N and C terminal:

NH₂-GGSKVFKSLPGQNEDLVLTSGG-COOH
(DNA: 5′-
GGGGGCTCTAAAGTTTTCAAAAGCCTCCCTGGGCAGAACGAAGATCTGGT
TCTGACCAGCGGGGGT-3′)

can be inserted into Herceptin light chain CDR 2 in between Tyr 49 and Phe 53. Stefin A loop 1 amino acid sequence was reversed: NH₂-LGGPI-COOH (DNA: 5′-GTCGTAGCGGGTACT-3′) and can be inserted into Herceptin light chain CDR 3 in between His 91 and Thr 93.

The amplified full-length insert and pBAD vector backbone are digested by NheI-HF and SalI-HF restriction enzymes (New England Biolabs, MA) at 37° C. for 3 hours, followed by DNA gel extraction. Using T4 DNA ligase (New England Biolabs, MA), the chimeric gene is in-frame ligated into pBAD backbone vector, resulting in bacterial expression vector of stefin A-derived antibody. The generated pBAD expression vector is confirmed by DNA sequencing.

### Example 8

### Expression and Purification of Stefin A-Derived Antibodies

The proteins are expressed in DH10B Escherichia coli (E. coli) transformed with pBAD expression vectors by electroporation. Overnight bacterial culture in LB-broth was diluted to 1 liter culture medium and grown at 37° C. until OD_{600 nm} of 0.6. The antibody expression is induced with 0.02% L-arabinose and grown at 25° C. overnight with a shaking speed of 180 rpm. Expressed antibody is secreted into periplasm. Cells are harvested 24 hours post induction. Bacterial periplasma is lysed with lysis buffer (20% sucrose, 30 mM Tris-HCl pH 8, 1 mM EDTA, and 0.2 mg ml⁻¹ lysozyme) for 1 hour while shaking at 180 rpm. After centrifugation at 15,000 rpm at 4° C. for 30 minutes, the supernatant is then loaded onto protein G chromatography (Thermo Fisher Scientific, IL) for purification of the expressed antibody. The purity of antibodies are examined by SDS-PAGE gels.

### Example 9

### Expression and Purification of Protease Propeptide-Fused Antibodies

Antibody fusion proteins are expressed in free-style HEK 293 cells transiently transfected with pFUSE expression vectors. The expressed proteins are secreted into culture media. HEK 293 suspension cells were cultured in freestyle 293 expression medium (Life Techonologies, CA) in shaker flasks at 37° C., 5% CO₂ and 125 rpm. To generate Herceptin light chain-CatB Propeptide fusion proteins, 20 μg of pFUSE vector of Herceptin heavy chain and 10 μg vector of Herceptin light chain-CatB propeptide fusion protein genes are combined and then mixed with 60 μl 293fectin (Life Technologies, CA) for transfection of 30 million of freestyle HEK 293 cells. To produce Herceptin heavy chain-CatB propeptide fusion proteins, 10 μg of vector expressing Herceptin light chain and 20 μg of vector expressing Herceptin heavy chain-CatB propeptide fusion proteins are combined for transfection. The culture media is collected at 48 hours and 96 hours after transfection then and subjected to Protein G chromatography (Thermo Fisher Scientific, IL). The purity of antibodies are examined by SDS-PAGE gels.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

SEQUENCE LISTING

Human Cathepsin B Peptide Sequence
CGSCWAFGAV EAISDRICIH TNVSVEVSAE DLLTCCGSMC GDGCNGGYPA
EAWNFWTRKG LVSGGLYESH VGCRPYSIPP CEHHVNGSRP PCTGEGDTPK
CSKICEPGYS PTYKQDKHYG YDSYSVSNSE KDIMAEIYKN GPVEGAFSVY
SDFLLYKSGV YQHVTGEMMG GHAIRILGWG VENGTPYWLV ANSWN
Stefin A cDNA

| | |
|---|---|
| 1 | actttggttc cagcatcctg tccagcaaag aagcaatcag ccaaaatgat acctggaggc |
| 61 | ttatctgagg ccaaacccgc cactccagaa atccaggaga ttgttgataa ggttaaacca |
| 121 | cagcttgaag aaaaaacaaa tgagacttac ggaaaattgg aagctgtgca gtataaaact |
| 181 | caagttgttg ctggaacaaa ttactacatt aaggtacgag caggtgataa taaatatatg |
| 241 | cacttgaaag tattcaaaag tcttcccgga caaaatgagg acttggtact tactggatac |
| 301 | caggttgaca aaaacaagga tgacgagctg acgggctttt agcagcatgt acccaaagtg |
| 361 | ttctgattcc ttcaactggc tactgagtca tgatccttgc tgataaatat aaccatcaat |
| 421 | aaagaagcat tcttttccaa aaaaaaaaaa aaaagaaaaa aaaaaaaaaa aaaaagtggc |
| 481 | gctgggcagc gcgggtccca accagaaacc cgcacaggcg ac |

Stefin A Peptide Sequence
MIPGGLSEAKPATPEIQEIVDKVKPQLEEKTNETYGKLEAVQYKTQVVAGTNYYIKV
RAGDNKYMHLKVFKSLPGQNEDLVLTGYQVDKNKDDELTGF
Herceptin Heavy Chain polypeptide
EVQLVESGGG LVQPGGSLRL SCAASGFNIK DTYIHWVRQA
PGKGLEWVAR IYPTNGYTRY ADSVKGRFTI SADTSKNTAY LQMNSLRAED
TAVYYCSRWGGDGFYAMDYW GQGTLVTVSS ASTKGPSVFP LAPSSKSTSG
GTAALGCLVK DYFPEPVTVSWNSGALTSGV HTFPAVLQSS GLYSLSSVVT
VPSSSLGTQT YICNVNHKPS NTKVDKKVEPKSCDKTHTCP PCPAPELLGG
PSVFLFPPKP KDTLMISRTP EVTCVVVDVS HEDPEVKFNWYVDGVEVHNA
KTKPREEQYN STYRVVSVLT VLHQDWLNGK EYKCKVSNKA LPAPIEKTIS
KAKGQPREPQ VYTLPPSREE MTKNQVSLTC LVKGFYPSDI AVEWESNGQP
ENNYKTTPPVLDSDGSFFLY SKLTVDKSRW QQGNVFSCSV MHEALHNHYT
QKSLSLSPG
Herceptin Light Chain polypeptide
DIQMTQSPSS LSASVGDRVT ITCRASQDVN TAVAWYQQKP
GKAPKLLIYS ASFLYSGVPS RFSGSRSGTD FTLTISSLQP EDFATYYCQQ
HYTTPPTFGQGTKVEIKRTV AAPSVFIFPP SDEQLKSGTA SVVCLLNNFY
PREAKVQWKV DNALQSGNSQESVTEQDSKD STYSLSSTLT LSKADYEKHK
VYACEVTHQG LSSPVTKSFN RGEC
Stefin A loop 1 polypeptide
H₂N-LGGPI-COOH
Stefin A loop 1 polynucleotide
CTGGGAGGTCCGATT
Stefin A loop 2 polypeptide
H₂N-VVAGT-COOH
Stefin A loop 2 polynucleotide
GTCGTAGCGGGTACT
Stefin A loop 3 polypeptide
H₂N-GGSKSLPGQNEDLSGG-COOH
Stefin A loop 3 polynucleotide
GGGGGCTCTAAAAGCCTCCCTGGGCAGAACGAAGATCTGAGCGGGGGT
Linker
GGS
Propeptide polypeptide
H₂N-
RSRPSFHPLSDELVNYVNKRNTTWQAGHNFYNVDMSYLKRLCGTFLGGPKPPQRVM
FTEDL-COOH
Propeptide polynucleotide
CGTTCTCGTCCGTCTTTCCACCCGCTGTCTGACGAACTGGTTAACTACGTTAACAA
ACGTAACACCACCTGGCAGGCTGGTCACAACTTCTACAACGTTGACATGTCTTAC
CTGAAACGTCTGTGCGGTACCTTCCTGGGTGGTCCGAAACCGCCGCAGCGTGTTA
TGTTCACCGAAGACCTG
Stefin A Coil-Coil “stalk” ascending polypeptide
H₂N-GGSGAKLAALKAKLAALK-COOH
Stefin A Coil-Coil “stalk” ascending polynucleotide
GGCGGAAGCGGAGCAAAGCTCGCCGCACTGAAAGCCAAGCTGGCCGCTCTGAAG
Stefin A Coil-Coil “stalk” descending strand polypeptide
H₂N-ELAALEAELAALEAGGSG-COOH
Stefin A Coil-Coil “stalk” descending strand polynucleotide
GAACTGGCCGCACTGGAAGCTGAGCTGGCTGCCCTCGAAGCTGGAGGCTCTGGA
[[NOTE: In a number of the following sequences amino acids from Stefin A or propeptide
(respectively) are bolded and GGS linkers are underlined]]
Stefin A derived antibody Light Chain polypeptide
DIQMTQSPSSLSASVGDRVTITCRASQDVNVVAGTTAVAWYQQKPGKAPKLLIYGG
SKSLPGQNEDLSGGFLYSGVPSRFSGSRSGTDFTLTISSLQPEDFATYYCQQHLGGPI
TTPPTFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTK
SFNRGEC
Stefin A derived antibody Heavy Chain polynucleotide
gacatccagatgacccagtctccatcctccctgtctgcatctgtaggagacagagtcaccatcacttgccgggcaagtcaggatgtgaa
tGTCGTAGCGGGTACTaccgcggtcgcatggtatcagcagaaaccagggaaagcccctaagctcctgatctatGG
GGGCTCTAAAAGCCTCCCTGGGCAGAACGAAGATCTGAGCGGGGGTttcttgtatagt
ggggtcccatcaaggttcagtggcagtagatctgggacagatttcactctcaccatcagcagtctgcaacctgaagattttgcaacttact
actgtcaacagcatCTGGGAGGTCCGATTactacccctccgacgttcggccaaggtaccaagcttgagatcaaacga
actgtggctgcaccatctgtatcatcttcccgccatctgatgagcagttgaaatctggaactgcctctgtcgtgtgcctgctgaataacttc
tatcccagagaggccaaagtacagtggaaggtggataacgccctccaatcgggtaactcccaggagagtgtcacagagcaggaca
gcaaggacagcacctacagcctcagcagcaccctgacgctgagcaaagcagactacgagaaacacaaagtctacgcctgcgaagt
cacccatcagggcctgtcctcgcccgtcacaaagagcttcaacaggggagagtgt
Stefin A derived antibody Heavy Chain polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHT
Stefin A derived antibody Heavy Chain polynucleotide
Gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgagcccaaatcttgtgacaaaactcacaca
Propeptide-antibody fusion Light Chain polypeptide
RSRPSFHPLSDELVNYVNKRNTTWQAGHNFYNVDMSYLKRLCGTFLGGPKPPQ
RVMFTEDLDIQMTQSPSSLSASVGDRVTITCRASQDVNTAVAWYQQKPGKAPKLLI
YSASFLYSGVPSRFSGSRSGTDFTLTISSLQPEDFATYYCQQHYTTPPTFGQGTKLEIK
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Propeptide-antibody fusion Light Chain polynucleotide
CGTTCTCGTCCGTCTTTCCACCCGCTGTCTGACGAACTGGTTAACTACGTTA
ACAAACGTAACACCACCTGGCAGGCTGGTCACAACTTCTACAACGTTGACAT
GTCTTACCTGAAACGTCTGTGCGGTACCTTCCTGGGTGGTCCGAAACCGCC
GCAGCGTGTTATGTTCACCGAAGACCTGgacatccagatgacccagtctccatcctccctgtctgcatct
gtaggagacagagtcaccatcacttgccgggcaagtcaggatgtgaataccgcggtcgcatggtatcagcagaaaccagggaaagc
ccctaagctcctgatctattctgcatccttcttgtatagtggggtcccatcaaggttcagtggcagtagatctgggacagatttcactctcac
catcagcagtctgcaacctgaagattttgcaacttactactgtcaacagcattacactacccctccgacgttcggccaaggtaccaagctt
gagatcaaacgaactgtggctgcaccatctgtcttcatcttcccgccatctgatgagcagttgaaatctggaactgcctctgtcgtgtgcc
tgctgaataacttctatcccagagaggccaaagtacagtggaaggtggataacgccctccaatcgggtaactcccaggagagtgtcac
agagcaggacagcaaggacagcacctacagcctcagcagcaccctgacgctgagcaaagcagactacgagaaacacaaagtcta
cgcctgcgaagtcacccatcagggcctgtcctcgcccgtcacaaagagcttcaacaggggagagtgt
Propeptide-antibody fusion Heavy Chain polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
Propeptide-antibody fusion Heavy Chain polynucleotide
gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgaacccaaatcttgcgacaaaactcacacatgcccaccgtgcccag
cacctgaactcctggggggaccgtcagtcttcctcttccccccaaaacccaaggacaccctcatgatctcccggacccctgaggtcac
atgcgtggtggtggacgtgagccacgaagaccctgaggtcaagttcaactggtacgtggacggcgtggaggtgcataatgccaaga
caaagccgcgggaggagcagtacaacagcacgtaccgtgtggtcagcgtcctcaccgtcctgcaccaggactggctgaatggcaa
ggagtacaagtgcaaggtctccaacaaagccctcccagcccccatcgagaaaaccatctccaaagccaaagggcagccccgagaa
ccacaggtgtacaccctgcccccatcccgggatgagctgaccaagaaccaggtcagcctgacctgcctggtcaaaggcttctatccc
agcgacatcgccgtggagtgggagagcaatgggcagccggagaacaactacaagaccacgcctcccgtgctggactccgacggc
tccttcttcctctacagcaagctcaccgtggacaagagcaggtggcagcaggggaacgtcttctcatgctccgtgatgcatgaggctct
gcacaaccactacacgcagaagagcctctccctgtctccgggtaaa
Gen1 FAb VL polypeptide
DIQMTQSPSSLSASVGDRVTITCRASQDVNVVAGTTAVAWYQQKPGKAPKLLIYGG
SKSLPGQNEDLSGGFLYSGVPSRFSGSRSGTDFTLTISSLQPEDFATYYCQQHLGGPI
TTPPTFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTK
SFNRGEC
Gen1 FAb VL polynucleotide
gacatccagatgacccagtctccatcctccctgtctgcatctgtaggagacagagtcaccatcacttgccgggcaagtcaggatgtgaa
tGTCGTAGCGGGTACTaccgcggtcgcatggtatcagcagaaaccagggaaagcccctaagctcctgatctatGG
GGGCTCTAAAAGCCTCCCTGGGCAGAACGAAGATCTGAGCGGGGGTttcttgtatagt
ggggtcccatcaaggttcagtggcagtagatctgggacagatttcactctcaccatcagcagtctgcaacctgaagattttgcaacttact
actgtcaacagcatCTGGGAGGTCCGATTactacccctccgacgttcggccaaggtaccaagcttgagatcaaacga
actgtggctgcaccatctgtatcatcttcccgccatctgatgagcagttgaaatctggaactgcctctgtcgtgtgcctgctgaataacttc
tatcccagagaggccaaagtacagtggaaggtggataacgccctccaatcgggtaactcccaggagagtgtcacagagcaggaca
gcaaggacagcacctacagcctcagcagcaccctgacgctgagcaaagcagactacgagaaacacaaagtctacgcctgcgaagt
cacccatcagggcctgtcctcgcccgtcacaaagagcttcaacaggggagagtgt
Gen1 FAb VH polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHT
Gen1 FAb VH polynucleotide
Gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgagcccaaatcttgtgacaaaactcacaca
Propeptide N fusion VL polypeptide
RSRPSFHPLSDELVNYVNKRNTTWQAGHNFYNVDMSYLKRLCGTFLGGPKPPQ
RVMFTEDLDIQMTQSPSSLSASVGDRVTITCRASQDVNTAVAWYQQKPGKAPKLLI
YSASFLYSGVPSRFSGSRSGTDFTLTISSLQPEDFATYYCQQHYTTPPTFGQGTKLEIK
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC
Propeptide N fusion VL polynucleotide
CGTTCTCGTCCGTCTTTCCACCCGCTGTCTGACGAACTGGTTAACTACGTTA
ACAAACGTAACACCACCTGGCAGGCTGGTCACAACTTCTACAACGTTGACAT
GTCTTACCTGAAACGTCTGTGCGGTACCTTCCTGGGTGGTCCGAAACCGCC
GCAGCGTGTTATGTTCACCGAAGACCTGgacatccagatgacccagtctccatcctccctgtctgcatct
gtaggagacagagtcaccatcacttgccgggcaagtcaggatgtgaataccgcggtcgcatggtatcagcagaaaccagggaaagc
ccctaagctcctgatctattctgcatccttcttgtatagtggggtcccatcaaggttcagtggcagtagatctgggacagatttcactctcac
catcagcagtctgcaacctgaagattttgcaacttactactgtcaacagcattacactacccctccgacgttcggccaaggtaccaagctt
gagatcaaacgaactgtggctgcaccatctgtcttcatcttcccgccatctgatgagcagttgaaatctggaactgcctctgtcgtgtgcc
tgctgaataacttctatcccagagaggccaaagtacagtggaaggtggataacgccctccaatcgggtaactcccaggagagtgtcac
agagcaggacagcaaggacagcacctacagcctcagcagcaccctgacgctgagcaaagcagactacgagaaacacaaagtcta
cgcctgcgaagtcacccatcagggcctgtcctcgcccgtcacaaagagcttcaacaggggagagtgt
Propeptide N fusion VH with FC (normal VH) polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
Propeptide N fusion VH with FC (normal VH) polynucleotide
gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgaacccaaatcttgcgacaaaactcacacatgcccaccgtgcccag
cacctgaactcctggggggaccgtcagtcttcctcttccccccaaaacccaaggacaccctcatgatctcccggacccctgaggtcac
atgcgtggtggtggacgtgagccacgaagaccctgaggtcaagttcaactggtacgtggacggcgtggaggtgcataatgccaaga
caaagccgcgggaggagcagtacaacagcacgtaccgtgtggtcagcgtcctcaccgtcctgcaccaggactggctgaatggcaa
ggagtacaagtgcaaggtctccaacaaagccctcccagcccccatcgagaaaaccatctccaaagccaaagggcagccccgagaa
ccacaggtgtacaccctgcccccatcccgggatgagctgaccaagaaccaggtcagcctgacctgcctggtcaaaggcttctatccc
agcgacatcgccgtggagtgggagagcaatgggcagccggagaacaactacaagaccacgcctcccgtgctggactccgacggc
tccttcttcctctacagcaagctcaccgtggacaagagcaggtggcagcaggggaacgtcttctcatgctccgtgatgcatgaggctct
gcacaaccactacacgcagaagagcctctccctgtctccgggtaaa
Stefin A Loop X Light Chain polypeptide
DIQMTQSPSSLSASVGDRVTITCRASQDVNTAVAWYQQKPGKAPKLLIYGGSKSLP
GQNEDLSGGFLYSGVPSRFSGSRKIYYNTGAVVQTKYQFTLTISSLQPEDFATYYCQ
QHYTTPPTFGQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSS
PVTKSFNRGEC
Stefin A Loop X Light Chain polynucleotide
gacatccagatgacccagtctccatcctccctgtctgcatctgtaggagacagagtcaccatcacttgccgggcaagtcaggatgtgaa
taccgcggtcgcatggtatcagcagaaaccagggaaagcccctaagctcctgatctatGGGGGCTCTAAAAGCCTC
CCTGGGCAGAACGAAGATCTGAGCGGGGGTttcttgtatagtggggtcccatcaaggttcagtggcagta
gaAAGATATATTACAATACTGGAGCGGTTGTGCAAACCAAGTATCAAttcactctcaccat
cagcagtctgcaacctgaagattttgcaacttactactgtcaacagcattacactacccctccgacgttcggccaaggtaccaagcttga
gatcaaacgaactgtggctgcaccatctgtatcatcttcccgccatctgatgagcagttgaaatctggaactgcctctgtcgtgtgcctg
ctgaataacttctatcccagagaggccaaagtacagtggaaggtggataacgccctccaatcgggtaactcccaggagagtgtcaca
gagcaggacagcaaggacagcacctacagcctcagcagcaccctgacgctgagcaaagcagactacgagaaacacaaagtctac
gcctgcgaagtcacccatcagggcctgtcctcgcccgtcacaaagagcttcaacaggggagagtgt
Stefin A Loop X Heavy Chain polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHT
Stefin A Loop X Heavy Chain polynucleotide
Gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgagcccaaatcttgtgacaaaactcacaca
Stefin A Coil-Coil Light Chain polypeptide
DIQMTQSPSSLSASVGDRVTITCRASQGGSGAKLAALKAKLAALKGGGGSKTQVVA
GTNYYGGGGSELAALEAELAALEAGGSGNTAVAWYQQKPGKAPKLLIYSASFLYSG
VPSRFSGSRSGTDFTLTISSLQPEDFATYYCQQHGGSGAKLAALKAKLAALKGGGGS
KVFKSLPGQNEDLVLTGGGGSELAALEAELAALEAGGSGTPPTFGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
Stefin A Coil-Coil Light Chain polynucleotide
gacatccagatgacccagtctccatcctccctgtctgcatctgtaggagacagagtcaccatcacttgccgggcaagtcaggggggtt
ccggcgcgaagttagcggcattaaaagctaaactcgcggctctcaaaggtggaggtggcagcAAAACCCAGGTCGTA
GCGGGTACTAACTACTACggtggcggcggatcggaacttgctgcgttggaagcggaacttgcggcgctggaagcc
ggtgggagtggcaataccgcggtcgcatggtatcagcagaaaccagggaaagcccctaagctcctgatctattctgcatccttcttgta
tagtggggtcccatcaaggttcagtggcagtagatctgggacagatttcactctcaccatcagcagtctgcaacctgaagattttgcaac
ttactactgtcaacagcatggcggctctggagcaaaattggctgcattaaaggcgaaactggcagcactgaaaggtggcggtggtagt
AAAGTTTTCAAAAGCCTCCCTGGGCAGAACGAAGATCTGGTTCTGACCggcggaggcg
gatcggagctggcagccttggaagccgaactcgccgcacttgaagcgggaggtagcggcacccctccgacgttcggccaaggtac
caagcttgagatcaaacgaactgtggctgcaccatctgtcttcatcttcccgccatctgatgagcagttgaaatctggaactgcctctgtc
gtgtgcctgctgaataacttctatcccagagaggccaaagtacagtggaaggtggataacgccctccaatcgggtaactcccaggaga
gtgtcacagagcaggacagcaaggacagcacctacagcctcagcagcaccctgacgctgagcaaagcagactacgagaaacaca
aagtctacgcctgcgaagtcacccatcagggcctgtcctcgcccgtcacaaagagatcaacaggggagagtgt
Stefin A Coil-Coil Heavy Chain polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHT
Stefin A Coil-Coil Heavy Chain polynucleotide
Gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgagcccaaatcttgtgacaaaactcacaca
Stefin A CDR 23 Swap Light Chain polypeptide
DIQMTQSPSSLSASVGDRVTITCRASQDVNVVAGTTAVAWYQQKPGKAPKLLIYSLG
GPIASFLYSGVPSRFSGSRSGTDFTLTISSLQPEDFATYYCQQKSLPGQNEDLTFGQGT
KLEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
Stefin A CDR 2 3 Swap Light Chain polynucleotide
gacatccagatgacccagtctccatcctccctgtctgcatctgtaggagacagagtcaccatcacttgccgggcaagtcaggatgtgaa
tGTCGTAGCGGGTACTaccgcggtcgcatggtatcagcagaaaccagggaaagcccctaagctcctgatctattctCT
GGGAGGTCCGATTgcatccttcttgtatagtggggtcccatcaaggttcagtggcagtagatctgggacagatttcactctca
ccatcagcagtctgcaacctgaagattttgcaacttactactgtcaacagAAAAGCCTCCCTGGGCAGAACGAA
GATCTGacgttcggccaaggtaccaagcttgagatcaaacgaactgtggctgcaccatctgtcttcatcttcccgccatctgatga
gcagttgaaatctggaactgcctctgtcgtgtgcctgctgaataacttctatcccagagaggccaaagtacagtggaaggtggataacg
ccctccaatcgggtaactcccaggagagtgtcacagagcaggacagcaaggacagcacctacagcctcagcagcaccctgacgct
gagcaaagcagactacgagaaacacaaagtctacgcctgcgaagtcacccatcagggcctgtcctcgcccgtcacaaagagcttca
acaggggagagtgt
Stefin A CDR 23 Swap Heavy Chain polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHT
Stefin A CDR 2 3 Swap Heavy Chain polynucleotide
Gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgagcccaaatcttgtgacaaaactcacaca
Stefin A CDR 2 Ext Light Chain polypeptide
DIQMTQSPSSLSASVGDRVTITCRASQDVNVVAGTTAVAWYQQKPGKAPKLLIYGG
SKVFKSLPGQNEDLVLTSGGFLYSGVPSRFSGSRSGTDFTLTISSLQPEDFATYYCQQ
HLGGPITTPPTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGL
SSPVTKSFNRGEC
Stefin A CDR 2 Ext Light Chain polynucleotide
gacatccagatgacccagtctccatcctccctgtctgcatctgtaggagacagagtcaccatcacttgccgggcaagtcaggatgtgaa
tGTCGTAGCGGGTACTaccgcggtcgcatggtatcagcagaaaccagggaaagcccctaagctcctgatctatGGG
GGCTCTAAAGTTTTCAAAAGCCTCCCTGGGCAGAACGAAGATCTGGTTCTGACCA
GCGGGGGTttatgtatagtggggtcccatcaaggttcagtggcagtagatctgggacagatttcactctcaccatcagcagtctg
caacctgaagattttgcaacttactactgtcaacagcatCTGGGAGGTCCGATTactacccctccgacgttcggccaaggt
accaagcttgagatcaaacgaactgtggctgcaccatctgtcttcatcttcccgccatctgatgagcagttgaaatctggaactgcctctg
tcgtgtgcctgctgaataacttctatcccagagaggccaaagtacagtggaaggtggataacgccctccaatcgggtaactcccagga
gagtgtcacagagcaggacagcaaggacagcacctacagcctcagcagcaccctgacgctgagcaaagcagactacgagaaaca
caaagtctacgcctgcgaagtcacccatcagggcctgtcctcgcccgtcacaaagagcttcaacaggggagagtgt
Stefin A CDR 2 Ext Heavy Chain polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHT
Stefin A CDR 2 Ext Heavy Chain polynucleotide
gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgagcccaaatcttgtgacaaaactcacaca
Propeptide fused to heavy chain N terminal Light Chain polypeptide
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Propeptide fused to heavy chain N terminal Light Chain polynucleotide
gacatccagatgacccagtctccatcctccctgtctgcatctgtaggagacagagtcaccatcacttgccgggcaagtcaggatgtgaa
taccgcggtcgcatggtatcagcagaaaccagggaaagcccctaagctcctgatctattctgcatccttcttgtatagtggggtcccatc
aaggttcagtggcagtagatctgggacagatttcactctcaccatcagcagtctgcaacctgaagattttgcaacttactactgtcaacag
cattacactacccctccgacgttcggccaaggtaccaagcttgagatcaaacgaactgtggctgcaccatctgtcttcatcttcccgcca
tctgatgagcagttgaaatctggaactgcctctgtcgtgtgcctgctgaataacttctatcccagagaggccaaagtacagtggaaggtg
gataacgccctccaatcgggtaactcccaggagagtgtcacagagcaggacagcaaggacagcacctacagcctcagcagcaccc
tgacgctgagcaaagcagactacgagaaacacaaagtctacgcctgcgaagtcacccatcagggcctgtcctcgcccgtcacaaag
agcttcaacaggggagagtgt
Propeptide fused to heavy chain N terminal Heavy Chain polypeptide
RSRPSFHPLSDELVNYVNKRNTTWQAGHNFYNVDMSYLKRLCGTFLGGPKPPQRV
MFTEDLEVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWGGDGFYA
MDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHYTQKSLSL
SPGK
Propeptide fused to heavy chain N terminal Heavy Chain polynucleotide
CGTTCTCGTCCGTCTTTCCACCCGCTGTCTGACGAACTGGTTAACTACGTTAACAA
ACGTAACACCACCTGGCAGGCTGGTCACAACTTCTACAACGTTGACATGTCTTAC
CTGAAACGTCTGTGCGGTACCTTCCTGGGTGGTCCGAAACCGCCGCAGCGTGTTA
TGTTCACCGAAGACCTGgaggtgcagctggtggagtctggaggaggcttggtccagcctggggggtccctgagact
ctcctgtgcagcctctgggttcaatattaaggacacttacatccactgggtccgccaggctccagggaaggggctggagtgggtcgca
cgtatttatcctaccaatggttacacacgctacgcagactccgtgaagggccgattcaccatctccgcagacacttccaagaacacggc
gtatcttcaaatgaacagcctgagagccgaggacacggccgtgtattactgttcgagatggggcggtgacggcttctatgccatggact
actggggccaaggaaccctggtcaccgtctcctcagcctccaccaagggcccatcggtatccccctggcaccctcctccaagagca
cctctgggggcacagcggccctgggctgcctggtcaaggactacttccccgaaccggtgacggtgtcgtggaactcaggcgccctg
accagcggcgtgcacaccttcccggctgtcctacagtcctcaggactctactccctcagcagcgtggtgactgtgccctctagcagctt
gggcacccagacctacatctgcaacgtgaatcacaagcccagcaacaccaaggtggacaagaaagttgaacccaaatcttgcgaca
aaactcacacatgcccaccgtgcccagcacctgaactcctggggggaccgtcagtcttcctcttccccccaaaacccaaggacaccct
catgatctcccggacccctgaggtcacatgcgtggtggtggacgtgagccacgaagaccctgaggtcaagttcaactggtacgtgga
cggcgtggaggtgcataatgccaagacaaagccgcgggaggagcagtacaacagcacgtaccgtgtggtcagcgtcctcaccgtc
ctgcaccaggactggctgaatggcaaggagtacaagtgcaaggtctccaacaaagccctcccagcccccatcgagaaaaccatctc
caaagccaaagggcagccccgagaaccacaggtgtacaccctgcccccatcccgggatgagctgaccaagaaccaggtcagcct
gacctgcctggtcaaaggcttctatcccagcgacatcgccgtggagtgggagagcaatgggcagccggagaacaactacaagacc
acgcctcccgtgctggactccgacggctccttcttcctctacagcaagctcaccgtggacaagagcaggtggcagcaggggaacgtc
ttctcatgctccgtgatgcatgaggctctgcacaaccactacacgcagaagagcctctccctgtctccgggtaaa
Propeptide fused to heavy chain C terminal Light Chain polypeptide
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Propeptide fused to heavy chain C terminal Light Chain polynucleotide
gacatccagatgacccagtctccatcctccctgtctgcatctgtaggagacagagtcaccatcacttgccgggcaagtcaggatgtgaa
taccgcggtcgcatggtatcagcagaaaccagggaaagcccctaagctcctgatctattctgcatccttcttgtatagtggggtcccatc
aaggttcagtggcagtagatctgggacagatttcactctcaccatcagcagtctgcaacctgaagattttgcaacttactactgtcaacag
cattacactacccctccgacgttcggccaaggtaccaagcttgagatcaaacgaactgtggctgcaccatctgtcttcatcttcccgcca
tctgatgagcagttgaaatctggaactgcctctgtcgtgtgcctgctgaataacttctatcccagagaggccaaagtacagtggaaggtg
gataacgccctccaatcgggtaactcccaggagagtgtcacagagcaggacagcaaggacagcacctacagcctcagcagcaccc
tgacgctgagcaaagcagactacgagaaacacaaagtctacgcctgcgaagtcacccatcagggcctgtcctcgcccgtcacaaag
agcttcaacaggggagagtgt
Propeptide fused to heavy chain C terminal Heavy Chain polypeptide
EVQLVESGGGLVQPGGSLRLSCAASGFNIKDTYIHWVRQAPGKGLEWVARIYPTNG
YTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKVDKKVEPKSCD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSLSLSPGK
RSRPSFHPLSDELVNYVNKRNTTWQAGHNFYNVDMSYLKRLCGTFLGGPKPPQRV
MFTEDL
Propeptide fused to heavy chain C terminal Heavy Chain polynucleotide
gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagatggggcggtgacggcttctatgccatggactactggggccaaggaaccctggtcac
cgtctcctcagcctccaccaagggcccatcggtcttccccctggcaccctcctccaagagcacctctgggggcacagcggccctggg
ctgcctggtcaaggactacttccccgaaccggtgacggtgtcgtggaactcaggcgccctgaccagcggcgtgcacaccttcccggc
tgtcctacagtcctcaggactctactccctcagcagcgtggtgactgtgccctctagcagcttgggcacccagacctacatctgcaacgt
gaatcacaagcccagcaacaccaaggtggacaagaaagttgaacccaaatcttgcgacaaaactcacacatgcccaccgtgcccag
cacctgaactcctggggggaccgtcagtcttcctcttccccccaaaacccaaggacaccctcatgatctcccggacccctgaggtcac
atgcgtggtggtggacgtgagccacgaagaccctgaggtcaagttcaactggtacgtggacggcgtggaggtgcataatgccaaga
caaagccgcgggaggagcagtacaacagcacgtaccgtgtggtcagcgtcctcaccgtcctgcaccaggactggctgaatggcaa
ggagtacaagtgcaaggtctccaacaaagccctcccagcccccatcgagaaaaccatctccaaagccaaagggcagccccgagaa
ccacaggtgtacaccctgcccccatcccgggatgagctgaccaagaaccaggtcagcctgacctgcctggtcaaaggcttctatccc
agcgacatcgccgtggagtgggagagcaatgggcagccggagaacaactacaagaccacgcctcccgtgctggactccgacggc
tccttcttcctctacagcaagctcaccgtggacaagagcaggtggcagcaggggaacgtcttctcatgctccgtgatgcatgaggctct
gcacaaccactacacgcagaagagcctctccctgtctccgggtaaaCGTTCTCGTCCGTCTTTCCACCCGCTG
TCTGACGAACTGGTTAACTACGTTAACAAACGTAACACCACCTGGCAGGCTGGTC
ACAACTTCTACAACGTTGACATGTCTTACCTGAAACGTCTGTGCGGTACCTTCCT
GGGTGGTCCGAAACCGCCGCAGCGTGTTATGTTCACCGAAGACCTG
Propeptide fused to heavy chain CDR 3 Xa Light Chain polypeptide
DIQMTQSPSSLSASVGDRVTITCRASQDVNTAVAWYQQKPGKAPKLLIYSASFLYSG
VPSRFSGSRSGTDFTLTISSLQPEDFATYYCQQHYTTPPTFGQGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC
Propeptide fused to heavy chain CDR 3 Xa Light Chain polynucleotide
gacatccagatgacccagtctccatcctccctgtctgcatctgtaggagacagagtcaccatcacttgccgggcaagtcaggatgtgaa
taccgcggtcgcatggtatcagcagaaaccagggaaagcccctaagctcctgatctattctgcatccttcttgtatagtggggtcccatc
aaggttcagtggcagtagatctgggacagatttcactctcaccatcagcagtctgcaacctgaagattttgcaacttactactgtcaacag
cattacactacccctccgacgttcggccaaggtaccaagcttgagatcaaacgaactgtggctgcaccatctgtatcatcttcccgcca
tctgatgagcagttgaaatctggaactgcctctgtcgtgtgcctgctgaataacttctatcccagagaggccaaagtacagtggaaggtg
gataacgccctccaatcgggtaactcccaggagagtgtcacagagcaggacagcaaggacagcacctacagcctcagcagcaccc
tgacgctgagcaaagcagactacgagaaacacaaagtctacgcctgcgaagtcacccatcagggcctgtcctcgcccgtcacaaag
agcttcaacaggggagagtgt
Propeptide fused to heavy chain CDR 3 Xa Heavy Chain polypeptide
EVQLVESGGGLVQPGGSLRLSCAASGFNIKDTYIHWVRQAPGKGLEWVARTYPTNG
YTRYADSVKGRFTISADTSKNTAYLQMNSLRAEDTAVYYCSRGGSGAKLAALKAKL
AALKCGGGGSIEGRRSRPSFHPLSDELVNYVNKRNTTWQAGHNFYNVDMSYLKRLC
GTFLGGPKPPQRVMFTEDLGGGGSCELAALEAELAALEAGGSGDYWGQGTLVTVSS
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAP
PVAGPSVFLFPPKPKDTLMISRTPEVTCVVVDVSHEDPEVKFNWYVDGVEVHNAKT
KPREEQYNSTYRVVSVLTVLHQDWLNGKEYKCKVSNKGLPSSIEKTISKAKGQPREP
QVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK
Propeptide fused to heavy chain CDR 3 Xa Heavy Chain polynucleotide
gaggtgcagctggtggagtctggaggaggcttggtccagcctggggggtccctgagactctcctgtgcagcctctgggttcaatatta
aggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagc
cgaggacacggccgtgtattactgttcgagaGGCGGAAGCGGAGCAAAGCTCGCCGCACTGAAAG
CCAAGCTGGCCGCTCTGAAGTGCGGGGGTGGCGGAAGCatcgaaggtcgtCGTTCTCGT
CCGTCTTTCCACCCGCTGTCTGACGAACTGGTTAACTACGTTAACAAACGTAACA
CCACCTGGCAGGCTGGTCACAACTTCTACAACGTTGACATGTCTTACCTGAAACG
TCTGTGCGGTACCTTCCTGGGTGGTCCGAAACCGCCGCAGCGTGTTATGTTCACC
GAAGACCTGGGCGGAGGTGGGAGTTGCGAACTGGCCGCACTGGAAGCTGAGCTG
GCTGCCCTCGAAGCTGGAGGCTCTGGAgactactggggccaaggaaccctggtcaccgtctcctcagcctc
caccaagggcccatcggtcttccccctggcaccctcctccaagagcacctctgggggcacagcggccctgggctgcctggtcaagg
actacttccccgaaccggtgacggtgtcgtggaactcaggcgccctgaccagcggcgtgcacaccttcccggctgtcctacagtcctc
aggactctactccctcagcagcgtggtgactgtgccctctagcagcttgggcacccagacctacatctgcaacgtgaatcacaagccc
agcaacaccaaggtggacaagaaagttgaacccaaatcttgcgacaaaactcacacatgcccaccgtgcccagcacctCCaGtc
GCcggaccgtcagtcttcctcttcccTccaaaacccaaggacaccctcatgatctcccggacccctgaggtcacatgcgtggtggtg
gacgtgagccacgaagaccctgaggtcaagttcaactggtacgtggacggcgtggaggtgcataatgccaagacaaagccgcggg
aggagcagtacaacagcacgtaccgtgtggtcagcgtcctcaccgtcctgcaccaggactggctgaatggcaaggagtacaagtgc
aaggtctccaacaaagGcctcccaAGcTccatcgagaaaaccatctccaaagccaaagggcagccccgagaaccacaggtgta
caccctgccTccatcccgggatgagctgaccaagaaccaggtcagcctgacctgcctggtcaaaggcttctatcccagcgacatcgc
cgtggagtgggagagcaatgggcagccggagaacaactacaagaccacgcctcccgtgctggactccgacggctccttcttcctcta
cagcaagctcaccgtggacaagagcaggtggcagcaggggaacgtcttctcatgctccgtgatgcatgaggctctgcacaaccacta
cacgcagaagagcctctccctgtctccgggtaaa

1. A recombinant polynucleotide encoding an antibody fragment, comprising: 1) a polynucleotide encoding one, two or three inhibitory loop regions of stefin A incorporated into a the Fab complementary determining regions (CDRs) heavy or light chains of an anti-HER2 antibody or an equivalent thereof; or 2) a polynucleotide encoding one or more inhibitory loops of stefin A substituted for the Fab loops of an anti-HER2 antibody or an equivalent thereof.

2. The recombinant polynucleotide of claim 1, wherein the anti-HER2 antibody is Herceptin or an equivalent thereof.

3. The recombinant polynucleotide of claim 1, wherein the polynucleotide comprises: 1) a reversed stefin A loop 1 grafted between amino acid His91 and Thr93 in the light chain CDR3 of the anti-HER2 antibody; 2) the stefin A loop 2 inserted between the Asn30 and THR31 in the anti-HER2 antibody light chain CDR1; and the stefin A loop 3 and linkers at the N- and C-termini between Tyr49 and Phe53 in the anti-HER2 antibody light chain CDR2, or an equivalent of each thereof.

4. The recombinant polynucleotide of claim 1, wherein the polynucleotide comprises: 1) a reversed stefin A loop 1 grafted between amino acid Gly101 and Gly103 in the heavy chain CDR3 of the anti-HER2 antibody; 2) the stefin A loop 2 inserted between the Asp31 and Thr32 in the anti-HER2 antibody heavy chain CDR1; and the stefin A loop 3 and linkers at the N- and C-termini between Ile51 and Gly56 in the anti-HER2 antibody heavy chain CDR2, or an equivalent of each thereof.

5. The recombinant polynucleotide of claim 1, wherein an inhibitory loop region of stefin A loop 3, with an optional linker, is substituted into the CDR2 of the light chain between Tyr49 and Phe53 and the stefin loop 2 polynucleotide is reversed and inserted into a loop of the light chain between Arg66 and Phe71, or an equivalent of each thereof.

6. The recombinant polynucleotide of claim 1, wherein an inhibitory loop region of stefin A loop 2, with an optional coil-coil linkers at the N- and/or C-terminii, is substituted into the CDR1 of the light chain between Gln27 and Asn30 and the stefin A loop 3 polynucleotide, with an optionally coil-coil linkers at the N- and/or C-termini, and inserted into the CDR3 of between His91 and Thr94, or an equivalent of each thereof.

7. The recombinant polynucleotide of claim 1, wherein two inhibitory loop regions are substituted for an anti-HER2 antibody CDR loops of the light chain, wherein stefin A loop2, with an optional coil-coil linkers at the N- and/or C-terminii, is substituted into the CDR1 of the light chain between Gln27 and Asn30, and the stefin A loop 3 polynucleotide, with an optional coil-coil linkers at the N- and/or C-termini, and inserted into the light chain CDR3 of between His91 and Thr94, or an equivalent of each thereof.

8. The recombinant polynucleotide of claim 1, wherein three inhibitory loop regions are substituted for an anti-HER2 antibody Fab CDR loops of the light chain, wherein stefin A loop2 is substituted into the CDR1 of the light chain between Asn30 and Thr31, and the stefin A loop 1 polynucleotide, in reverse, and inserted into the light chain CDR2 between Ser50 and Ala51, and stefin A loop is inserted into light chain CDR 3 between Gln90 and Thr97, or an equivalent of each thereof.

9. The recombinant polynucleotide of claim 1, wherein three inhibitory loops of stefin A are substituted for the Fab CDR loops of the light chain anti-HER2 antibody fragment, wherein stefin A loop 2 is inserted into the light chain CDR 1 between Asn30 and Thr31, and stefin A loop 3 with an optional linker at its N- and C-termini, and inserted into light chain CDR 2 between Tyr49 and Phe53, and stefin A loop 1 is reversed and inserted into the light chain CDR 3 between His 91 and Thr93, or an equivalent of each thereof.

10. The recombinant polynucleotide of claim 1, wherein the stefin A loop 1 polynucleotide comprises CTGGGAGGTCCGATT (SEQ ID NO: 7)); and/or the stefin A loop 2 polynculeotide comprises GTCGTAGCGGGTACT (SEQ ID NO: 8)); and/or the Stefin A loop 3 polynucleotide comprises GGGGGCTCTAAAAGCCTCCCTGGGCAGAACGAAGATCTGAGCGGGGGT (SEQ ID NO: 9), or an equivalent of each thereof.

11.-17. (canceled)

18. A recombinant stefin A-derived antibody fragment comprising an inhibitory loop region of stefin A incorporated into the Fab complementary determining regions (CDRs) light chains or heavy chains of an anti-HER2 antibody or an equivalent or each thereof, wherein the antibody fragment is an expression product of a recombinant polynucleotide of claim 1.

19.-20. (canceled)

21. A recombinant anti-HER2 antibody comprising an antibody fragment of claim 18 and a N-terminal or C-terminal cathepsin B propeptide fusion polypeptide.

22. A recombinant polynucleotide encoding a propetide-fused anti-HER2 antibody fragment, comprising a polynucleotide encoding N-terminal or C-terminal propeptide cathepsin B fused to a polynucleotide encoding an anti-HER2 light or a heavy chain.

23. (canceled)

24. The recombinant polynucleotide of claim 22, wherein the cathepsin B propeptide polynucleotide encodes a polypeptide having the sequence H2N-RSRPSFHPLSDELVNYVNKRNTTWQAGHNFYNVDMSYLKRLCGTFLGGPKPPQRVMFTEDL-COOH (SEQ ID NO: 10).

25.-29. (canceled)

30. An isolated host cell comprising a vector encoding a stefin A substituted anti-HER2 antibody heavy chain fragment and a vector encoding a cathepsin B propeptide light chain or a cathepsin B propeptide heavy chain.

31.-39. (canceled)

40. A method for inhibiting human cathepsin B, comprising contacting the cathepsin B with an effective amount of the antibody prepared by growing the host cell of claim 30, under conditions to express the stefin A substituted anti-HER2 antibody heavy chain fragment and the cathepsin B propeptide light chain or a cathepsin B propeptide heavy chain 37.

41.-42. (canceled)

43. A method for one or more of: 1) treating a condition mediated by cathepsin B activity, 2) to inhibit and/or arrest cancer metastasis, or 3) reduce the size of tumor mass or masses in a subject in need thereof, comprising administering to the subject an effective amount of a recombinant cathepsin B antibody that inhibits proteolytic activity of human cathepsin B, or an equivalent thereof, wherein the antibody inhibits the proteolytic activity of human cathepsin B in a dose-dependent fashion.

44.-46. (canceled)

47. The method of claim 43, wherein the condition is cancer.

48. One or more isolated polynucleotides or polypeptides, comprising the sequences:
(SEQ ID NO: 11)
a) Stefin A-derived antibody Light Chain Amino Acid Sequence
DIQMTQSPSSLSASVGDRVTITCRASQDVNVVAGTTAVAWYQQKPGKAPKLLIY
GGSKSLPGQNEDLSGGFLYSGVPSRFSGSRSGTDFTLTISSLQPEDFATYYCQQHLGGPIT
TPPTFGQGTKLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRG
EC;
or
(SEQ ID NO: 12)
b) Stefin A-derived antibody Light Chain DNA Sequence
gacatccagatgacccagtctccatcctccctgtctgcatctgtaggagacagagtcaccatcacttgccgggcaagtcaggatg
tgaatGTCGTAGCGGGTACTaccgcggtcgcatggtatcagcagaaaccagggaaagcccctaagctcctgatctatGGG
GGCTCTAAAAGCCTCCCTGGGCAGAACGAAGATCTGAGCGGGGGTttcttgtatagtggggtccc
atcaaggttcagtggcagtagatctgggacagatttcactctcaccatcagcagtctgcaacctgaagattttgcaacttactactgtcaacag
catCTGGGAGGTCCGATTactacccctccgacgttcggccaaggtaccaagcttgagatcaaacgaactgtggctgcaccat
ctgtcttcatcttcccgccatctgatgagcagttgaaatctggaactgcctctgtcgtgtgcctgctgaataacttctatcccagagaggccaaa
gtacagtggaaggtggataacgccctccaatcgggtaactcccaggagagtgtcacagagcaggacagcaaggacagcacctacagcct
cagcagcaccctgacgctgagcaaagcagactacgagaaacacaaagtctacgcctgcgaagtcacccatcagggcctgtcctcgcccg
tcacaaagagcttcaacaggggagagtgt;
or
(SEQ ID NO: 13)
c) Stefin A-derived antibody Heavy Chain Amino Acid Sequence
EVQLVESGGGLVQPGGSLRLSCAASGFNIKDTYMWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHT;
or
(SEQ ID NO: 14)
d) Stefin A-derived antibody Heavy Chain DNA Sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgagcccaaatcttgtgacaaaactcacaca;
or
(SEQ ID NO: 15)
e) Propeptide-antibody fusion Light Chain Amino Acid Sequence
RSRPSFHPLSDELVNYVNKRNTTWQAGHNFYNVDMSYLKRLCGTFLGGPKPPQ
RVMFTEDLDIQMTQSPSSLSASVGDRVTITCRASQDVNTAVAWYQQKPGKAPKLLIYSA
SFLYSGVPSRFSGSRSGTDFTLTISSLQPEDFATYYCQQHYTTPPTFGQGTKLEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC;
or
(SEQ ID NO: 16)
f) Propeptide-antibody fusion Light Chain DNA Sequence
CGTTCTCGTCCGTCTTTCCACCCGCTGTCTGACGAACTGGTTAACTACGTTAA
CAAACGTAACACCACCTGGCAGGCTGGTCACAACTTCTACAACGTTGACATGTCTTA
CCTGAAACGTCTGTGCGGTACCTTCCTGGGTGGTCCGAAACCGCCGCAGCGTGTTAT
GTTCACCGAAGACCTGgacatccagatgacccagtctccatcctccctgtctgcatctgtaggagacagagtcacc atcactt
gccgggcaagtcaggatgtgaataccgcggtcgcatggtatcagcagaaaccagggaaagcccctaagctcctgatctattctgcatcctt
cttgtatagtggggtcccatcaaggttcagtggcagtagatctgggacagatttcactctcaccatcagcagtctgcaacctgaagattttgca
acttactactgtcaacagcattacactacccctccgacgttcggccaaggtaccaagcttgagatcaaacgaactgtggctgcaccatctgtc
ttcatcttcccgccatctgatgagcagttgaaatctggaactgcctctgtcgtgtgcctgctgaataacttctatcccagagaggccaaagtaca
gtggaaggtggataacgccctccaatcgggtaactcccaggagagtgtcacagagcaggacagcaaggacagcacctacagcctcagc
agcaccctgacgctgagcaaagcagactacgagaaacacaaagtctacgcctgcgaagtcacccatcagggcctgtcctcgcccgtcac
aaagagcttcaacaggggagagtgt;
or
(SEQ ID NO: 17)
g) Propeptide-antibody fusion Heavy Chain Amino Acid Sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK;
or
(SEQ ID NO: 18)
h) Propeptide-antibody fusion Heavy Chain DNA Sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgaacccaaatcttgcgacaaaactcacacatgcccaccgtgcccagcacctgaactcctggggg
gaccgtcagtcttcctcttccccccaaaacccaaggacaccctcatgatctcccggacccctgaggtcacatgcgtggtggtggacgtgag
ccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccggg
atgagctgaccaagaaccaggtcagcctgacctgcctggtcaaaggcttctatcccagcgacatcgccgtggagtgggagagcaatggg
cagccggagaacaactacaagaccacgcctcccgtgctggactccgacggctccttcttcctctacagcaagctcaccgtggacaagagc
aggtggcagcaggggaacgtcttctcatgctccgtgatgcatgaggctctgcacaaccactacacgcagaagagcctctccctgtctccgg
gtaaa;
or
(SEQ ID NO: 19)
i) Gen1 Fab VL Amino Acid Sequence
DIQMTQSPSSLSASVGDRVTITCRASQDVNVVAGTTAVAWYQQKPGKAPKLLIY
GGSKSLPGQNEDLSGGFLYSGVPSRFSGSRSGTDFTLTISSLQPEDFATYYCQQHLGGPIT
TPPTFGQGTKLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRG
EC;
(SEQ ID NO: 20)
j) Gen1 Fab VL DNA Sequence
gacatccagatgacccagtctccatcctccctgtctgcatctgtaggagacagagtcaccatcacttgccgggcaagtcaggatg
tgaatGTCGTAGCGGGTACTaccgcggtcgcatggtatcagcagaaaccagggaaagcccctaagctcctgatctatGGG
GGCTCTAAAAGCCTCCCTGGGCAGAACGAAGATCTGAGCGGGGGTttcttgtatagtggggtccc
atcaaggttcagtggcagtagatctgggacagatttcactctcaccatcagcagtctgcaacctgaagattttgcaacttactactgtcaacag
catCTGGGAGGTCCGATTactacccctccgacgttcggccaaggtaccaagcttgagatcaaacgaactgtggctgcaccat
ctgtcttcatcttcccgccatctgatgagcagttgaaatctggaactgcctctgtcgtgtgcctgctgaataacttctatcccagagaggccaaa
gtacagtggaaggtggataacgccctccaatcgggtaactcccaggagagtgtcacagagcaggacagcaaggacagcacctacagcct
cagcagcaccctgacgctgagcaaagcagactacgagaaacacaaagtctacgcctgcgaagtcacccatcagggcctgtcctcgcccg
tcacaaagagcttcaacaggggagagtgt;
(SEQ ID NO: 21)
k) Gen1 Fab VH Amino Acid Sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHT;
(SEQ ID NO: 22)
l) Gen1 Fab VH DNA Sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgagcccaaatcttgtgacaaaactcacaca;
(SEQ ID NO: 23)
m) Propeptide N fusion VL Amino Acid Sequence
RSRPSFHPLSDELVNYVNKRNTTWQAGHNFYNVDMSYLKRLCGTFLGGPKPPQ
RVMFTEDLDIQMTQSPSSLSASVGDRVTITCRASQDVNTAVAWYQQKPGKAPKLLIYSA
SFLYSGVPSRFSGSRSGTDFTLTISSLQPEDFATYYCQQHYTTPPTFGQGTKLEIKRTVAA
PSVFIFPPSDEQLKSGTASVVCLLNNFYPREAKVQWKVDNALQSGNSQESVTEQDSKDS
TYSLSSTLTLSKADYEKHKVYACEVTHQGLSSPVTKSFNRGEC;
(SEQ ID NO: 24)
n) Propeptide N fusion VL DNA Sequence
CGTTCTCGTCCGTCTTTCCACCCGCTGTCTGACGAACTGGTTAACTACGTTAA
CAAACGTAACACCACCTGGCAGGCTGGTCACAACTTCTACAACGTTGACATGTCTTA
CCTGAAACGTCTGTGCGGTACCTTCCTGGGTGGTCCGAAACCGCCGCAGCGTGTTAT
GTTCACCGAAGACCTGgacatccagatgacccagtctccatcctccctgtctgcatctgtaggagacagagtcaccatcactt
gccgggcaagtcaggatgtgaataccgcggtcgcatggtatcagcagaaaccagggaaagcccctaagctcctgatctattctgcatcctt
cttgtatagtggggtcccatcaaggttcagtggcagtagatctgggacagatttcactctcaccatcagcagtctgcaacctgaagattttgca
acttactactgtcaacagcattacactacccctccgacgttcggccaaggtaccaagcttgagatcaaacgaactgtggctgcaccatctgtc
ttcatcttcccgccatctgatgagcagttgaaatctggaactgcctctgtcgtgtgcctgctgaataacttctatcccagagaggccaaagtaca
gtggaaggtggataacgccctccaatcgggtaactcccaggagagtgtcacagagcaggacagcaaggacagcacctacagcctcagc
agcaccctgacgctgagcaaagcagactacgagaaacacaaagtctacgcctgcgaagtcacccatcagggcctgtcctcgcccgtcac
aaagagcttcaacaggggagagtgt;
(SEQ ID NO: 25)
o) Propeptide N fusion VH with FC Amino Acid Sequence (normal VH)
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK;
(SEQ ID NO: 26)
p) Propeptide N fusion VH with FC DNA Sequence (normal VH)
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgaacccaaatcttgcgacaaaactcacacatgcccaccgtgcccagcacctgaactcctggggg
gaccgtcagtcttcctcttccccccaaaacccaaggacaccctcatgatctcccggacccctgaggtcacatgcgtggtggtggacgtgag
ccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccggg
atgagctgaccaagaaccaggtcagcctgacctgcctggtcaaaggcttctatcccagcgacatcgccgtggagtgggagagcaatggg
cagccggagaacaactacaagaccacgcctcccgtgctggactccgacggctccttcttcctctacagcaagctcaccgtggacaagagc
aggtggcagcaggggaacgtatctcatgctccgtgatgcatgaggctctgcacaaccactacacgcagaagagcctctccctgtctccgg
gtaaa;
(SEQ ID NO: 27)
q) Stefin A Loop X LC amino acid sequence
DIQMTQSPSSLSASVGDRVTITCRASQDVNTAVAWYQQKPGKAPKLLIYGGSKS
LPGQNEDLSGGFLYSGVPSRFSGSRKIYYNTGAVVQTKYQFTLTISSLQPEDFATYYCQQ
HYTTPPTFGQGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSF
NRGEC;
(SEQ ID NO: 28)
r) Stefin A Loop X LC DNA sequence
gacatccagatgacccagtctccatcctccctgtctgcatctgtaggagacagagtcaccatcacttgccgggcaagtcaggatg
tgaataccgcggtcgcatggtatcagcagaaaccagggaaagcccctaagctcctgatctatGGGGGCTCTAAAAGCCTC
CCTGGGCAGAACGAAGATCTGAGCGGGGGTttcttgtatagtggggtcccatcaaggttcagtggcagtagaA
AGATATATTACAATACTGGAGCGGTTGTGCAAACCAAGTATCAAttcactctcaccatcagcagt
ctgcaacctgaagattttgcaacttactactgtcaacagcattacactacccctccgacgttcggccaaggtaccaagcttgagatcaaacga
actgtggctgcaccatctgtcttcatcttcccgccatctgatgagcagttgaaatctggaactgcctctgtcgtgtgcctgctgaataacttctat
cccagagaggccaaagtacagtggaaggtggataacgccctccaatcgggtaactcccaggagagtgtcacagagcaggacagcaagg
acagcacctacagcctcagcagcaccctgacgctgagcaaagcagactacgagaaacacaaagtctacgcctgcgaagtcacccatcag
ggcctgtcctcgcccgtcacaaagagcttcaacaggggagagtgt;
(SEQ ID NO: 29)
s) Stefin A Loop X Heavy chain amino acid sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHT;
(SEQ ID NO: 30)
t) Stefin A Loop X Heavy chain DNA sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgagcccaaatcttgtgacaaaactcacaca;
(SEQ ID NO: 31)
u) Stefin A Coil-Coil LC amino acid sequence
DIQMTQSPSSLSASVGDRVTITCRASQGGSGAKLAALKAKLAALKGGGGSKTQV
VAGTNYYGGGGSELAALEAELAALEAGGSGNTAVAWYQQKPGKAPKLLIYSASFLYSG
VPSRFSGSRSGTDFTLTISSLQPEDFATYYCQQHGGSGAKLAALKAKLAALKGGGGSKV
FKSLPGQNEDLVLTGGGGSELAALEAELAALEAGGSGTPPTFGQGTKLEIKRTVAAPSVF
IFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC;
(SEQ ID NO: 32)
v) Stefin A Coil-Coil LC DNA sequence
gacatccagatgacccagtctccatcctccctgtctgcatctgtaggagacagagtcaccatcacttgccgggcaagtcagggg
ggttccggcgcgaagttagcggcattaaaagctaaactcgcggctctcaaaggtggaggtggcagcAAAACCCAGGTCGTA
GCGGGTACTAACTACTACggtggcggcggatcggaacttgctgcgttggaagcggaacttgcggcgctggaagccggt
gggagtggcaataccgcggtcgcatggtatcagcagaaaccagggaaagcccctaagctcctgatctattctgcatccttcttgtatagtgg
ggtcccatcaaggttcagtggcagtagatctgggacagatttcactctcaccatcagcagtctgcaacctgaagattttgcaacttactactgtc
aacagcatggcggctctggagcaaaattggctgcattaaaggcgaaactggcagcactgaaaggtggcggtggtagtAAAGTTTT
CAAAAGCCTCCCTGGGCAGAACGAAGATCTGGTTCTGACCggcggaggcggatcggagctggcag
ccttggaagccgaactcgccgcacttgaagcgggaggtagcggcacccctccgacgttcggccaaggtaccaagcttgagatcaaacga
actgtggctgcaccatctgtcttcatcttcccgccatctgatgagcagttgaaatctggaactgcctctgtcgtgtgcctgctgaataacttctat
cccagagaggccaaagtacagtggaaggtggataacgccctccaatcgggtaactcccaggagagtgtcacagagcaggacagcaagg
acagcacctacagcctcagcagcaccctgacgctgagcaaagcagactacgagaaacacaaagtctacgcctgcgaagtcacccatcag
ggcctgtcctcgcccgtcacaaagagcttcaacaggggagagtgt;
(SEQ ID NO: 33)
w) Stefin A Coil-Coil Heavy chain amino acid sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHT;
(SEQ ID NO: 34)
x) Stefin A Coil-Coil Heavy chain DNA sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgagcccaaatcttgtgacaaaactcacaca;
(SEQ ID NO: 35)
y) Stefin A CDR 2 3 Swap LC amino acid sequence
DIQMTQSPSSLSASVGDRVTITCRASQDVNVVAGTTAVAWYQQKPGKAPKLLIY
SLGGPIASFLYSGVPSRFSGSRSGTDFTLTISSLQPEDFATYYCQQKSLPGQNEDLTFGQG
TKLEIKRTVAAPSVFIFPPSDEQLKSGTASVVCLLNNFYPREAKVQWKVDNALQSGNSQ
ESVTEQDSKDSTYSLSSTLTLSKADYEKHKVYACEVTHQGLSSPVTKSFNRGEC;
(SEQ ID NO: 36)
z) Stefin A CDR 2 3 Swap LC DNA sequence
gacatccagatgacccagtctccatcctccctgtctgcatctgtaggagacagagtcaccatcacttgccgggcaagtcaggatg
tgaatGTCGTAGCGGGTACTaccgcggtcgcatggtatcagcagaaaccagggaaagcccctaagctcctgatctattctCT
GGGAGGTCCGATTgcatccttatgtatagtggggtcccatcaaggttcagtggcagtagatctgggacagatttcactctcacca
tcagcagtctgcaacctgaagattttgcaacttactactgtcaacagAAAAGCCTCCCTGGGCAGAACGAAGATC
TGacgttcggccaaggtaccaagcttgagatcaaacgaactgtggctgcaccatctgtcttcatcttcccgccatctgatgagcagttgaaa
tctggaactgcctctgtcgtgtgcctgctgaataacttctatcccagagaggccaaagtacagtggaaggtggataacgccctccaatcggg
taactcccaggagagtgtcacagagcaggacagcaaggacagcacctacagcctcagcagcaccctgacgctgagcaaagcagactac
gagaaacacaaagtctacgcctgcgaagtcacccatcagggcctgtcctcgcccgtcacaaagagcttcaacaggggagagtgt;
(SEQ ID NO: 37)
aa) Stefin A CDR 2 3 Swap Heavy chain amino acid sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHT;
(SEQ ID NO: 38)
bb) Stefin A CDR 2 3 Swap Heavy chain DNA sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgagcccaaatcttgtgacaaaactcacaca;
(SEQ ID NO: 39)
cc) Stefin A CDR 2 Ext LC amino acid sequence
DIQMTQSPSSLSASVGDRVTITCRASQDVNVVAGTTAVAWYQQKPGKAPKLLIY
GGSKVFKSLPGQNEDLVLTSGGFLYSGVPSRFSGSRSGTDFTLTISSLQPEDFATYYCQQ
HLGGPITTPPTFGQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEVTHQGLSSPV
TKSFNRGEC;
(SEQ ID NO: 40)
dd) Stefin A CDR 2 Ext LC DNA sequence
gacatccagatgacccagtctccatcctccctgtctgcatctgtaggagacagagtcaccatcacttgccgggcaagtcaggatg
tgaatGTCGTAGCGGGTACTaccgcggtcgcatggtatcagcagaaaccagggaaagcccctaagctcctgatctatGGG
GGCTCTAAAGTTTTCAAAAGCCTCCCTGGGCAGAACGAAGATCTGGTTCTGACCAGC
GGGGGTttatgtatagtggggtcccatcaaggttcagtggcagtagatctgggacagatttcactctcaccatcagcagtctgcaacct
gaagattttgcaacttactactgtcaacagcatCTGGGAGGTCCGATTactacccctccgacgttcggccaaggtaccaagctt
gagatcaaacgaactgtggctgcaccatctgtcttcatcttcccgccatctgatgagcagttgaaatctggaactgcctctgtcgtgtgcctgct
gaataacttctatcccagagaggccaaagtacagtggaaggtggataacgccctccaatcgggtaactcccaggagagtgtcacagagca
ggacagcaaggacagcacctacagcctcagcagcaccctgacgctgagcaaagcagactacgagaaacacaaagtctacgcctgcgaa
gtcacccatcagggcctgtcctcgcccgtcacaaagagcttcaacaggggagagtgt;
(SEQ ID NO: 41)
ee) Stefin A CDR 2 Ext Heavy chain amino acid sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHT;
(SEQ ID NO: 42)
ff) Stefin A CDR 2 Ext Heavy chain DNA sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgagcccaaatcttgtgacaaaactcacaca;
(SEQ ID NO: 43)
gg) Propeptide fused to heavy chain N terminal LC amino acid sequence
DIQMTQSPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC;
(SEQ ID NO: 44)
hh) Propeptide fused to heavy chain N terminal LC DNA sequence
gacatccagatgacccagtctccatcctccctgtctgcatctgtaggagacagagtcaccatcacttgccgggcaagtcaggatg
tgaataccgcggtcgcatggtatcagcagaaaccagggaaagcccctaagctcctgatctattctgcatccttcttgtatagtggggtcccatc
aaggttcagtggcagtagatctgggacagatttcactctcaccatcagcagtctgcaacctgaagattttgcaacttactactgtcaacagcatt
acactacccctccgacgttcggccaaggtaccaagcttgagatcaaacgaactgtggctgcaccatctgtcttcatcttcccgccatctgatg
agcagttgaaatctggaactgcctctgtcgtgtgcctgctgaataacttctatcccagagaggccaaagtacagtggaaggtggataacgcc
ctccaatcgggtaactcccaggagagtgtcacagagcaggacagcaaggacagcacctacagcctcagcagcaccctgacgctgagca
aagcagactacgagaaacacaaagtctacgcctgcgaagtcacccatcagggcctgtcctcgcccgtcacaaagagcttcaacagggga
gagtgt;
(SEQ ID NO: 45)
ii) Propeptide fused to heavy chain N terminal HC amino acid sequence
RSRPSFHPLSDELVNYVNKRNTTWQAGHNFYNVDMSYLKRLCGTFLGGPKPPQ
RVMFTEDLEVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWGGDGFYAM
DYWGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSCDKT
HTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK;
(SEQ ID NO: 46)
jj) Propeptide fused to heavy chain N terminal HC DNA sequence
CGTTCTCGTCCGTCTTTCCACCCGCTGTCTGACGAACTGGTTAACTACGTTAA
CAAACGTAACACCACCTGGCAGGCTGGTCACAACTTCTACAACGTTGACATGTCTTA
CCTGAAACGTCTGTGCGGTACCTTCCTGGGTGGTCCGAAACCGCCGCAGCGTGTTAT
GTTCACCGAAGACCTGgaggtgcagctggtggagtctggaggaggcttggtccagcctggggggtccctgagactctcct
gtgcagcctctgggttcaatattaaggacacttacatccactgggtccgccaggctccagggaaggggctggagtgggtcgcacgtatttat
cctaccaatggttacacacgctacgcagactccgtgaagggccgattcaccatctccgcagacacttccaagaacacggcgtatcttcaaat
gaacagcctgagagccgaggacacggccgtgtattactgttcgagatggggcggtgacggcttctatgccatggactactggggccaagg
aaccctggtcaccgtctcctcagcctccaccaagggcccatcggtcttccccctggcaccctcctccaagagcacctctgggggcacagc
ggccctgggctgcctggtcaaggactacttccccgaaccggtgacggtgtcgtggaactcaggcgccctgaccagcggcgtgcacacctt
cccggctgtcctacagtcctcaggactctactccctcagcagcgtggtgactgtgccctctagcagcttgggcacccagacctacatctgca
acgtgaatcacaagcccagcaacaccaaggtggacaagaaagttgaacccaaatcttgcgacaaaactcacacatgcccaccgtgccca
gcacctgaactcctggggggaccgtcagtcttcctcttccccccaaaacccaaggacaccctcatgatctcccggacccctgaggtcacat
gcgtggtggtggacgtgagccacgaagaccctgaggtcaagttcaactggtacgtggacggcgtggaggtgcataatgccaagacaaag
ccgcgggaggagcagtacaacagcacgtaccgtgtggtcagcgtcctcaccgtcctgcaccaggactggctgaatggcaaggagtacaa
gtgcaaggtctccaacaaagccctcccagcccccatcgagaaaaccatctccaaagccaaagggcagccccgagaaccacaggtgtac
accctgcccccatcccgggatgagctgaccaagaaccaggtcagcctgacctgcctggtcaaaggcttctatcccagcgacatcgccgtg
gagtgggagagcaatgggcagccggagaacaactacaagaccacgcctcccgtgctggactccgacggctccttcttcctctacagcaa
gctcaccgtggacaagagcaggtggcagcaggggaacgtcttctcatgctccgtgatgcatgaggctctgcacaaccactacacgcagaa
gagcctctccctgtctccgggtaaa;
(SEQ ID NO: 47)
kk) Propeptide fused to heavy chain C terminal LC amino acid sequence
DIQMTQSPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC;
(SEQ ID NO: 48)
ll) Propeptide fused to heavy chain C terminal LC DNA sequence
gacatccagatgacccagtctccatcctccctgtctgcatctgtaggagacagagtcaccatcacttgccgggcaagtcaggatg
tgaataccgcggtcgcatggtatcagcagaaaccagggaaagcccctaagctcctgatctattctgcatccttcttgtatagtggggtcccatc
aaggttcagtggcagtagatctgggacagatttcactctcaccatcagcagtctgcaacctgaagattttgcaacttactactgtcaacagcatt
acactacccctccgacgttcggccaaggtaccaagcttgagatcaaacgaactgtggctgcaccatctgtcttcatcttcccgccatctgatg
agcagttgaaatctggaactgcctctgtcgtgtgcctgctgaataacttctatcccagagaggccaaagtacagtggaaggtggataacgcc
ctccaatcgggtaactcccaggagagtgtcacagagcaggacagcaaggacagcacctacagcctcagcagcaccctgacgctgagca
aagcagactacgagaaacacaaagtctacgcctgcgaagtcacccatcagggcctgtcctcgcccgtcacaaagagcttcaacagggga
gagtgt;
(SEQ ID NO: 49)
mm) Propeptide fused to heavy chain C terminal HC amino acid sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRWGGDGFYAMDYW
GQGTLVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGKRSRPSFHPLSDELV
NYVNKRNTTWQAGHNFYNVDMSYLKRLCGTFLGGPKPPQRVMFTEDL;
(SEQ ID NO: 50)
nn) Propeptide fused to heavy chain C terminal HC DNA sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagatggggcggtgacggcttctatgccatggactactggggccaaggaaccctggtcaccgtctcc
tcagcctccaccaagggcccatcggtcttccccctggcaccctcctccaagagcacctctgggggcacagcggccctgggctgcctggtc
aaggactacttccccgaaccggtgacggtgtcgtggaactcaggcgccctgaccagcggcgtgcacaccttcccggctgtcctacagtcct
caggactctactccctcagcagcgtggtgactgtgccctctagcagcttgggcacccagacctacatctgcaacgtgaatcacaagcccag
caacaccaaggtggacaagaaagttgaacccaaatcttgcgacaaaactcacacatgcccaccgtgcccagcacctgaactcctggggg
gaccgtcagtcttcctcttccccccaaaacccaaggacaccctcatgatctcccggacccctgaggtcacatgcgtggtggtggacgtgag
ccacgaagaccctgaggtcaagttcaactggtacgtggacggcgtggaggtgcataatgccaagacaaagccgcgggaggagcagtac
aacagcacgtaccgtgtggtcagcgtcctcaccgtcctgcaccaggactggctgaatggcaaggagtacaagtgcaaggtctccaacaaa
gccctcccagcccccatcgagaaaaccatctccaaagccaaagggcagccccgagaaccacaggtgtacaccctgcccccatcccggg
atgagctgaccaagaaccaggtcagcctgacctgcctggtcaaaggcttctatcccagcgacatcgccgtggagtgggagagcaatggg
cagccggagaacaactacaagaccacgcctcccgtgctggactccgacggctccttcttcctctacagcaagctcaccgtggacaagagc
aggtggcagcaggggaacgtatctcatgctccgtgatgcatgaggctctgcacaaccactacacgcagaagagcctctccctgtctccgg
gtaaaCGTTCTCGTCCGTCTTTCCACCCGCTGTCTGACGAACTGGTTAACTACGTTAACA
AACGTAACACCACCTGGCAGGCTGGTCACAACTTCTACAACGTTGACATGTCTTACC
TGAAACGTCTGTGCGGTACCTTCCTGGGTGGTCCGAAACCGCCGCAGCGTGTTATGT
TCACCGAAGACCTG;
(SEQ ID NO: 51)
oo) Propeptide fused to heavy chain CDR 3 Xa LC amino acid sequence
DIQMTQSPSSLSASVGDRVTITCRASQDVNTAVAWYQQKPGKAPKLLIYSASFLY
SGVPSRFSGSRSGTDFTLTISSLQPEDFATYYCQQHYTTPPTFGQGTKLEIKRTVAAPSVFI
FPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC;
(SEQ ID NO: 52)
pp) Propeptide fused to heavy chain CDR 3 Xa LC DNA sequence
gacatccagatgacccagtctccatcctccctgtctgcatctgtaggagacagagtcaccatcacttgccgggcaagtcaggatg
tgaataccgcggtcgcatggtatcagcagaaaccagggaaagcccctaagctcctgatctattctgcatccttcttgtatagtggggtcccatc
aaggttcagtggcagtagatctgggacagatttcactctcaccatcagcagtctgcaacctgaagattttgcaacttactactgtcaacagcatt
acactacccctccgacgttcggccaaggtaccaagcttgagatcaaacgaactgtggctgcaccatctgtcttcatcttcccgccatctgatg
agcagttgaaatctggaactgcctctgtcgtgtgcctgctgaataacttctatcccagagaggccaaagtacagtggaaggtggataacgcc
ctccaatcgggtaactcccaggagagtgtcacagagcaggacagcaaggacagcacctacagcctcagcagcaccctgacgctgagca
aagcagactacgagaaacacaaagtctacgcctgcgaagtcacccatcagggcctgtcctcgcccgtcacaaagagcttcaacagggga
gagtgt;
(SEQ ID NO: 53)
qq) Propeptide fused to heavy chain CDR 3 Xa HC amino acid sequence
EVQLVESGGGLVQPGGSLRLSCAASGFNIKDTYIHWVRQAPGKGLEWVARIYPT
NGYTRYADSVKGRFTISADTSKNTAYLQMNSLRAEDTAVYYCSRGGSGAKLAALKAK
LAALKCGGGGSIEGRRSRPSFHPLSDELVNYVNKRNTTWQAGHNFYNVDMSYLKRLCG
TFLGGPKPPQRVMFTEDLGGGGSCELAALEAELAALEAGGSGDYWGQGTLVTVSSAST
KGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPPVAGPSV
FLFPPKPKDTLMISRTPEVTCVVVDVSHEDPEVKFNWYVDGVEVHNAKTKPREEQYNS
TYRVVSVLTVLHQDWLNGKEYKCKVSNKGLPSSIEKTISKAKGQPREPQVYTLPPSRDE
LTKNQVSLTCLVKGFYPSDIAVEWESNGQPENNYKTTPPVLDSDGSFFLYSKLTVDKSR
WQQGNVFSCSVMHEALHNHYTQKSLSLSPGK;
(SEQ ID NO: 54)
rr) Propeptide fused to heavy chain CDR 3 Xa HC DNA sequence
gaggtgcagctggtggagtctggaggaggcttggtccagcctggggggtccctgagactctcctgtgcagcctctgggttcaat
attaaggacacttacatccactgggtccgccaggctccagggaaggggctggagtgggtcgcacgtatttatcctaccaatggttacacacg
ctacgcagactccgtgaagggccgattcaccatctccgcagacacttccaagaacacggcgtatcttcaaatgaacagcctgagagccga
ggacacggccgtgtattactgttcgagaGGCGGAAGCGGAGCAAAGCTCGCCGCACTGAAAGCCAA
GCTGGCCGCTCTGAAGTGCGGGGGTGGCGGAAGCatcgaaggtcgtCGTTCTCGTCCGTCT
TTCCACCCGCTGTCTGACGAACTGGTTAACTACGTTAACAAACGTAACACCACCTGG
CAGGCTGGTCACAACTTCTACAACGTTGACATGTCTTACCTGAAACGTCTGTGCGGT
ACCTTCCTGGGTGGTCCGAAACCGCCGCAGCGTGTTATGTTCACCGAAGACCTGGGC
GGAGGTGGGAGTTGCGAACTGGCCGCACTGGAAGCTGAGCTGGCTGCCCTCGAAGC
TGGAGGCTCTGGAgactactggggccaaggaaccctggtcaccgtctcctcagcctccaccaagggcccatcggtcttccccc
tggcaccctcctccaagagcacctctgggggcacagcggccctgggctgcctggtcaaggactacttccccgaaccggtgacggtgtcgt
ggaactcaggcgccctgaccagcggcgtgcacaccttcccggctgtcctacagtcctcaggactctactccctcagcagcgtggtgactgt
gccctctagcagcttgggcacccagacctacatctgcaacgtgaatcacaagcccagcaacaccaaggtggacaagaaagttgaaccca
aatcttgcgacaaaactcacacatgcccaccgtgcccagcacctCCaGtcGCcggaccgtcagtatcctcttcccTccaaaacccaa
ggacaccctcatgatctcccggacccctgaggtcacatgcgtggtggtggacgtgagccacgaagaccctgaggtcaagttcaactggta
cgtggacggcgtggaggtgcataatgccaagacaaagccgcgggaggagcagtacaacagcacgtaccgtgtggtcagcgtcctcacc
gtcctgcaccaggactggctgaatggcaaggagtacaagtgcaaggtctccaacaaagGcctcccaAGcTccatcgagaaaaccatct
ccaaagccaaagggcagccccgagaaccacaggtgtacaccctgccTccatcccgggatgagctgaccaagaaccaggtcagcctga
cctgcctggtcaaaggcttctatcccagcgacatcgccgtggagtgggagagcaatgggcagccggagaacaactacaagaccacgcct
cccgtgctggactccgacggctccttcttcctctacagcaagctcaccgtggacaagagcaggtggcagcaggggaacgtcttctcatgct
ccgtgatgcatgaggctctgcacaaccactacacgcagaagagcctctccctgtctccgggtaaa;
or
ss) an equivalent of each thereof

49.-51. (canceled)

52. A method to express one or more polynucleotide(s) comprising growing a host cell comprising the polynucleotide of claim 48 under conditions to express the polynucleotide.

53.-54. (canceled)


SEQUENCE LISTING (86 sequences)

SEQ ID NO: 1 - protein, 195 aa, Homo sapiens
CGSCWAFGAVEAISDRICIHTNVSVEVSAEDLLTCCGSMCGDGCNGGYPAEAWNFWTRKGLVSGGLYESHVGCRPYSIPPCEHHVNGSRPPCTGEGDTPKCSKICEPGYSPTYKQDKHYGYDSYSVSNSEKDIMAEIYKNGPVEGAFSVYSDFLLYKSGVYQHVTGEMMGGHAIRILGWGVENGTPYWLVANSWN

SEQ ID NO: 2 - protein, 5 aa, Homo sapiens
QVVAG

SEQ ID NO: 3 - DNA, 522 bp, Homo sapiens
actttggttccagcatcctgtccagcaaagaagcaatcagccaaaatgatacctggaggcttatctgaggccaaacccgccactccagaaatccaggagattgttgataaggttaaaccacagcttgaagaaaaaacaaatgagacttacggaaaattggaagctgtgcagtataaaactcaagttgttgctggaacaaattactacattaaggtacgagcaggtgataataaatatatgcacttgaaagtattcaaaagtcttcccggacaaaatgaggacttggtacttactggataccaggttgacaaaaacaaggatgacgagctgacgggcttttagcagcatgtacccaaagtgttctgattccttcaactggctactgagtcatgatccttgctgataaatataaccatcaataaagaagcattcttttccaaaaaaaaaaaaaaaagaaaaaaaaaaaaaaaaaaaagtggcgctgggcagcgcgggtcccaaccagaaacccgcacaggcgac

SEQ ID NO: 4 - protein, 98 aa, Homo sapiens
MIPGGLSEAKPATPEIQEIVDKVKPQLEEKTNETYGKLEAVQYKTQVVAGTNYYIKVRAGDNKYMHLKVFKSLPGQNEDLVLTGYQVDKNKDDELTGF

SEQ ID NO: 5 - protein, 449 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 15 bp, Artificial Sequence
  Description of Artificial Sequence Synthetic oligonucleotide
ctgggaggtccgatt

SEQ ID NO: 8 - DNA, 15 bp, Artificial Sequence
  Description of Artificial Sequence Synthetic oligonucleotide
gtcgtagcgggtact

SEQ ID NO: 9 - DNA, 48 bp, Artificial Sequence
  Description of Artificial Sequence Synthetic oligonucleotide
gggggctctaaaagcctccctgggcagaacgaagatctgagcgggggt

SEQ ID NO: 10 - protein, 61 aa, Homo sapiens
RSRPSFHPLSDELVNYVNKRNTTWQAGHNFYNVDMSYLKRLCGTFLGGPKPPQRVMFTEDL

SEQ ID NO: 11 - protein, 236 aa, Artificial Sequence
  Description of Artificial Sequence Synthetic polypeptide
DIQMTQSPSSLSASVGDRVTITCRASQDVNVVAGTTAVAWYQQKPGKAPKLLIYGGSKSLPGQNEDLSGGFLYSGVPSRFSGSRSGTDFTLTISSLQPEDFATYYCQQHLGGPITTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 708 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaatgtcgtagcgggtactaccgcggtcgcatggtatcagcagaaaccagggaaagcccctaagctcctgatctatgggggctctaaaagcctccctgggcagaacgaagatctgagcgggggtttcttgtatagtggggtcccatcaaggttcagtggcagtagatctgggacagatttcactctcaccatcagcagtctgcaacctgaagattttgcaacttactactgtcaacagcatctgggaggtccgattactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 13 - protein, 228 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 14 - DNA, 684 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacaca

SEQ ID NO: 15 - protein, 275 aa, Artificial Sequence
  Description of Artificial Sequence Synthetic polypeptide
RSRPSFHPLSDELVNYVNKRNTTWQAGHNFYNVDMSYLKRLCGTFLGGPKPPQRVMFTEDLDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - DNA, 825 bp, Artificial Sequence
  Description of Artificial Sequence Synthetic polynucleotide
cgttctcgtccgtctttccacccgctgtctgacgaactggttaactacgttaacaaacgtaacaccacctggcaggctggtcacaacttctacaacgttgacatgtcttacctgaaacgtctgtgcggtaccttcctgggtggtccgaaaccgccgcagcgtgttatgttcaccgaagacctggacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 17 - protein, 450 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 1350 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 19 - protein, 236 aa, Artificial Sequence; identical to SEQ ID NO: 11
  Description of Artificial Sequence Synthetic polypeptide

SEQ ID NO: 20 - DNA, 708 bp, Artificial Sequence; identical to SEQ ID NO: 12
  Description of Artificial Sequence Synthetic polynucleotide

SEQ ID NO: 21 - protein, 228 aa, Artificial Sequence; identical to SEQ ID NO: 13
  Description of Artificial Sequence Synthetic polypeptide

SEQ ID NO: 22 - DNA, 684 bp, Artificial Sequence; identical to SEQ ID NO: 14
  Description of Artificial Sequence Synthetic polynucleotide

SEQ ID NO: 23 - protein, 275 aa, Artificial Sequence; identical to SEQ ID NO: 15
  Description of Artificial Sequence Synthetic polypeptide

SEQ ID NO: 24 - DNA, 825 bp, Artificial Sequence; identical to SEQ ID NO: 16
  Description of Artificial Sequence Synthetic polynucleotide

SEQ ID NO: 25 - protein, 450 aa, Artificial Sequence; identical to SEQ ID NO: 17
  Description of Artificial Sequence Synthetic polypeptide

SEQ ID NO: 26 - DNA, 1350 bp, Artificial Sequence; identical to SEQ ID NO: 18
  Description of Artificial Sequence Synthetic polynucleotide

SEQ ID NO: 27 - protein, 238 aa, Artificial Sequence
  Description of Artificial Sequence Synthetic polypeptide
DIQMTQSPSSLSASVGDRVTITCRASQDVNTAVAWYQQKPGKAPKLLIYGGSKSLPGQNEDLSGGFLYSGVPSRFSGSRKIYYNTGAVVQTKYQFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - DNA, 714 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctatgggggctctaaaagcctccctgggcagaacgaagatctgagcgggggtttcttgtatagtggggtcccatcaaggttcagtggcagtagaaagatatattacaatactggagcggttgtgcaaaccaagtatcaattcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 29 - protein, 228 aa, Artificial Sequence; identical to SEQ ID NO: 13
  Description of Artificial Sequence Synthetic polypeptide

SEQ ID NO: 30 - DNA, 684 bp, Artificial Sequence; identical to SEQ ID NO: 14
  Description of Artificial Sequence Synthetic polynucleotide

SEQ ID NO: 31 - protein, 329 aa, Artificial Sequence
  Description of Artificial Sequence Synthetic polypeptide
DIQMTQSPSSLSASVGDRVTITCRASQGGSGAKLAALKAKLAALKGGGGSKTQVVAGTNYYGGGGSELAALEAELAALEAGGSGNTAVAWYQQKPGKAPKLLIYSASFLYSGVPSRFSGSRSGTDFTLTISSLQPEDFATYYCQQHGGSGAKLAALKAKLAALKGGGGSKVFKSLPGQNEDLVLTGGGGSELAALEAELAALEAGGSGTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - DNA, 987 bp, Artificial Sequence
  Description of Artificial Sequence Synthetic polynucleotide
gacatccagatgacccagtctccatcctccctgtctgcatctgtaggagacagagtcaccatcacttgccgggcaagtcaggggggttccggcgcgaagttagcggcattaaaagctaaactcgcggctctcaaaggtggaggtggcagcaaaacccaggtcgtagcgggtactaactactacggtggcggcggatcggaacttgctgcgttggaagcggaacttgcggcgctggaagccggtgggagtggcaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcatggcggctctggagcaaaattggctgcattaaaggcgaaactggcagcactgaaaggtggcggtggtagtaaagttttcaaaagcctccctgggcagaacgaagatctggttctgaccggcggaggcggatcggagctggcagccttggaagccgaactcgccgcacttgaagcgggaggtagcggcacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 33 - protein, 228 aa, Artificial Sequence; identical to SEQ ID NO: 13
  Description of Artificial Sequence Synthetic polypeptide

SEQ ID NO: 34 - DNA, 684 bp, Artificial Sequence; identical to SEQ ID NO: 14
  Description of Artificial Sequence Synthetic polynucleotide

SEQ ID NO: 35 - protein, 228 aa, Artificial Sequence
  Description of Artificial Sequence Synthetic polypeptide
DIQMTQSPSSLSASVGDRVTITCRASQDVNVVAGTTAVAWYQQKPGKAPKLLIYSLGGPIASFLYSGVPSRFSGSRSGTDFTLTISSLQPEDFATYYCQQKSLPGQNEDLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - DNA, 684 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaatgtcgtagcgggtactaccgcggtcgcatggtatcagcagaaaccagggaaagcccctaagctcctgatctattctctgggaggtccgattgcatccttcttgtatagtggggtcccatcaaggttcagtggcagtagatctgggacagatttcactctcaccatcagcagtctgcaacctgaagattttgcaacttactactgtcaacagaaaagcctccctgggcagaacgaagatctgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 37 - protein, 228 aa, Artificial Sequence; identical to SEQ ID NO: 13
  Description of Artificial Sequence Synthetic polypeptide

SEQ ID NO: 38 - DNA, 684 bp, Artificial Sequence; identical to SEQ ID NO: 14
  Description of Artificial Sequence Synthetic polynucleotide

SEQ ID NO: 39 - protein, 242 aa, Artificial Sequence
  Description of Artificial Sequence Synthetic polypeptide
DIQMTQSPSSLSASVGDRVTITCRASQDVNVVAGTTAVAWYQQKPGKAPKLLIYGGSKVFKSLPGQNEDLVLTSGGFLYSGVPSRFSGSRSGTDFTLTISSLQPEDFATYYCQQHLGGPITTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 726 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaatgtcgtagcgggtactaccgcggtcgcatggtatcagcagaaaccagggaaagcccctaagctcctgatctatgggggctctaaagttttcaaaagcctccctgggcagaacgaagatctggttctgaccagcgggggtttcttgtatagtggggtcccatcaaggttcagtggcagtagatctgggacagatttcactctcaccatcagcagtctgcaacctgaagattttgcaacttactactgtcaacagcatctgggaggtccgattactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 41 - protein, 228 aa, Artificial Sequence; identical to SEQ ID NO: 13
  Description of Artificial Sequence Synthetic polypeptide

SEQ ID NO: 42 - DNA, 684 bp, Artificial Sequence; identical to SEQ ID NO: 14
  Description of Artificial Sequence Synthetic polynucleotide

SEQ ID NO: 43 - protein, 214 aa, Artificial Sequence
  Description of Artificial Sequence Synthetic polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - DNA, 642 bp, Artificial Sequence
  Description of Artificial Sequence Synthetic polynucleotide
gacatccagatgacccagtctccatcctccctgtctgcatctgtaggagacagagtcaccatcacttgccgggcaagtcaggatgtgaataccgcggtcgcatggtatcagcagaaaccagggaaagcccctaagctcctgatctattctgcatccttcttgtatagtggggtcccatcaaggttcagtggcagtagatctgggacagatttcactctcaccatcagcagtctgcaacctgaagattttgcaacttactactgtcaacagcattacactacccctccgacgttcggccaaggtaccaagcttgagatcaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgt

SEQ ID NO: 45 - protein, 511 aa, Artificial Sequence
  Description of Artificial Sequence Synthetic polypeptide
RSRPSFHPLSDELVNYVNKRNTTWQAGHNFYNVDMSYLKRLCGTFLGGPKPPQRVMFTEDLEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - DNA, 1533 bp, Artificial Sequence
  Description of Artificial Sequence Synthetic polynucleotide
cgttctcgtccgtctttccacccgctgtctgacgaactggttaactacgttaacaaacgtaacaccacctggcaggctggtcacaacttctacaacgttgacatgtcttacctgaaacgtctgtgcggtaccttcctgggtggtccgaaaccgccgcagcgtgttatgttcaccgaagacctggaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - protein, 214 aa, Artificial Sequence; identical to SEQ ID NO: 43
  Description of Artificial Sequence Synthetic polypeptide

SEQ ID NO: 48 - DNA, 642 bp, Artificial Sequence; identical to SEQ ID NO: 44
  Description of Artificial Sequence Synthetic polynucleotide

SEQ ID NO: 49 - protein, 511 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRSRPSFHPLSDELVNYVNKRNTTWQAGHNFYNVDMSYLKRLCGTFLGGPKPPQRVMFTEDL

SEQ ID NO: 50 - DNA, 1533 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagatggggcggtgacggcttctatgccatggactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaacgttctcgtccgtctttccacccgctgtctgacgaactggttaactacgttaacaaacgtaacaccacctggcaggctggtcacaacttctacaacgttgacatgtcttacctgaaacgtctgtgcggtaccttcctgggtggtccgaaaccgccgcagcgtgttatgttcaccgaagacctg

SEQ ID NO: 51 - protein, 214 aa, Artificial Sequence; identical to SEQ ID NO: 43
  Description of Artificial Sequence Synthetic polypeptide

SEQ ID NO: 52 - DNA, 642 bp, Artificial Sequence; identical to SEQ ID NO: 44
  Description of Artificial Sequence Synthetic polynucleotide

SEQ ID NO: 53 - protein, 553 aa, Artificial Sequence
  Description of Artificial Sequence Synthetic polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRGGSGAKLAALKAKLAALKCGGGGSIEGRRSRPSFHPLSDELVNYVNKRNTTWQAGHNFYNVDMSYLKRLCGTFLGGPKPPQRVMFTEDLGGGGSCELAALEAELAALEAGGSGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1659 bp, Artificial Sequence
  Description of Artificial Sequence Synthetic polynucleotide
gaggtgcagctggtggagtctggaggaggcttggtccagcctggggggtccctgagactctcctgtgcagcctctgggttcaatattaaggacacttacatccactgggtccgccaggctccagggaaggggctggagtgggtcgcacgtatttatcctaccaatggttacacacgctacgcagactccgtgaagggccgattcaccatctccgcagacacttccaagaacacggcgtatcttcaaatgaacagcctgagagccgaggacacggccgtgtattactgttcgagaggcggaagcggagcaaagctcgccgcactgaaagccaagctggccgctctgaagtgcgggggtggcggaagcatcgaaggtcgtcgttctcgtccgtctttccacccgctgtctgacgaactggttaactacgttaacaaacgtaacaccacctggcaggctggtcacaacttctacaacgttgacatgtcttacctgaaacgtctgtgcggtaccttcctgggtggtccgaaaccgccgcagcgtgttatgttcaccgaagacctgggcggaggtgggagttgcgaactggccgcactggaagctgagctggctgccctcgaagctggaggctctggagactactggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 55 - protein, 449 aa, Artificial Sequence; identical to SEQ ID NO: 5
  Description of Artificial Sequence Synthetic polypeptide

SEQ ID NO: 56 - protein, 214 aa, Artificial Sequence; identical to SEQ ID NO: 6
  Description of Artificial Sequence Synthetic polypeptide

SEQ ID NO: 57 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 58 - protein, 330 aa, Homo sapiens [sequence omitted]

SEQ ID NO: 59 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 60 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 61 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 62 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 63 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 64 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 65 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 5 aa, Artificial Sequence
  Description of Artificial Sequence Synthetic peptide
LGGPI

SEQ ID NO: 67 - protein, 5 aa, Artificial Sequence
  Description of Artificial Sequence Synthetic peptide
VVAGT

SEQ ID NO: 68 - protein, 16 aa, Artificial Sequence
  Description of Artificial Sequence Synthetic peptide
GGSKSLPGQNEDLSGG

SEQ ID NO: 69 - DNA, 183 bp, Homo sapiens
cgttctcgtccgtctttccacccgctgtctgacgaactggttaactacgttaacaaacgtaacaccacctggcaggctggtcacaacttctacaacgttgacatgtcttacctgaaacgtctgtgcggtaccttcctgggtggtccgaaaccgccgcagcgtgttatgttcaccgaagacctg

SEQ ID NO: 70 - protein, 18 aa, Artificial Sequence
  Description of Artificial Sequence Synthetic peptide
GGSGAKLAALKAKLAALK

SEQ ID NO: 71 - protein, 18 aa, Artificial Sequence
  Description of Artificial Sequence Synthetic peptide
ELAALEAELAALEAGGSG

SEQ ID NO: 72 - DNA, 54 bp, Artificial Sequence
  Description of Artificial Sequence Synthetic oligonucleotide
ggcggaagcggagcaaagctcgccgcactgaaagccaagctggccgctctgaag

SEQ ID NO: 73 - DNA, 54 bp, Artificial Sequence
  Description of Artificial Sequence Synthetic oligonucleotide
gaactggccgcactggaagctgagctggctgccctcgaagctggaggctctgga

SEQ ID NO: 74 - protein, 4 aa, Unknown
  Description of Unknown Factor Xa
IEGR

SEQ ID NO: 75 - protein, 19 aa, Artificial Sequence
  Description of Artificial Sequence Synthetic peptide
GGSGAKLAALKAKLAALKC

SEQ ID NO: 76 - protein, 19 aa, Artificial Sequence
  Description of Artificial Sequence Synthetic peptide
CELAALEAELAALEAGGSG

SEQ ID NO: 77 - protein, 15 aa, Artificial Sequence
  Description of Artificial Sequence Synthetic peptide
KIYYNTGAVVQTKYQ

SEQ ID NO: 78 - DNA, 45 bp, Artificial Sequence
  Description of Artificial Sequence Synthetic oligonucleotide
aagatatattacaatactggagcggttgtgcaaaccaagtatcaa

SEQ ID NO: 79 - protein, 57 aa, Artificial Sequence
  Description of Artificial Sequence Synthetic polypeptide
GGSGAKLAALKAKLAALKGGGGSKTQVVAGTNYYGGGGSELAALEAELAALEAGGSG

SEQ ID NO: 80 - DNA, 171 bp, Artificial Sequence
  Description of Artificial Sequence Synthetic polynucleotide
gggggttccggcgcgaagttagcggcattaaaagctaaactcgcggctctcaaaggtggaggtggcagcaaaacccaggtcgtagcgggtactaactactacggtggcggcggatcggaacttgctgcgttggaagcggaacttgcggcgctggaagccggtgggagtggc

SEQ ID NO: 81 - protein, 62 aa, Artificial Sequence
  Description of Artificial Sequence Synthetic polypeptide
GGSGAKLAALKAKLAALKGGGGSKVFKSLPGQNEDLVLTGGGGSELAALEAELAALEAGGSG

SEQ ID NO: 82 - DNA, 186 bp, Artificial Sequence
  Description of Artificial Sequence Synthetic polynucleotide
ggcggctctggagcaaaattggctgcattaaaggcgaaactggcagcactgaaaggtggcggtggtagtaaagttttcaaaagcctccctgggcagaacgaagatctggttctgaccggcggaggcggatcggagctggcagccttggaagccgaactcgccgcacttgaagcgggaggtagcggc

SEQ ID NO: 83 - protein, 10 aa, Artificial Sequence
  Description of Artificial Sequence Synthetic peptide
KSLPGQNEDL

SEQ ID NO: 84 - DNA, 30 bp, Artificial Sequence
  Description of Artificial Sequence Synthetic oligonucleotide
aaaagcctccctgggcagaacgaagatctg

SEQ ID NO: 85 - protein, 22 aa, Artificial Sequence
  Description of Artificial Sequence Synthetic peptide
GGSKVFKSLPGQNEDLVLTSGG

SEQ ID NO: 86 - DNA, 66 bp, Artificial Sequence
  Description of Artificial Sequence Synthetic oligonucleotide
gggggctctaaagttttcaaaagcctccctgggcagaacgaagatctggttctgaccagcgggggt